(19) United States
(12) Patent Application Publication
(10) Pub. No.: US 2026/0306848 A1
(43) Pub. Date: Oct. 8, 2026

(54) SYSTEM FOR RESPIRATORY THERAPY

(71) Applicant: ZOLL Medical Corporation, Chelmsford, MA (US)

(72) Inventors: Robin Germany, Dayton, MN (US); Kristofer James, Eagan, MN (US); Randy Westlund, Saint Paul, MN (US); Gary Freeman, Waltham, MA (US); Todd Goblish, Maple Grove, MN (US)

(73) Assignee: ZOLL Medical Corporation, Chelmsford, MA (US)

(21) Appl. No.: 19/567,939

(22) Filed: Mar. 16, 2026

Related U.S. Application Data
(60) Provisional application No. 63/783,735, filed on Apr. 4, 2025.

(51) Int. Cl. A61N 1/36; A61N 1/372

(52) U.S. Cl. CPC A61N 1/3611; A61N 1/36135; A61N 1/36157; A61N 1/36171; A61N 1/36175; A61N 1/37217

(57) ABSTRACT

An implantable pulse generator (IPG) is configured to provide respiratory conditioning for a patient via phrenic nerve stimulation. At least one processor is configured to determine that a patient is in a non-sleep time period and enter a patient control operational mode that provides one or more respiration conditioning sessions. Each respective respiration conditioning session corresponds to a pre-programmed conditioning stimulation profile specifying stimulation parameters for the respective respiration conditioning session. The processor is also configured to enter a sleep operational mode, the sleep operational mode having one or more sleep treatment sessions corresponding to a pre-programmed sleep stimulation profile, and control delivery of the electrical stimulation to the phrenic nerve according to at least one profile to modulate patient respiration during the sleep operational mode, based on a patient therapy mode indicator comprising an atrial fibrillation therapy indicator.

### CROSS-REFERENCE TO RELATED APPLICATIONS

This application claims priority under 35 U.S.C. § 119 (e) to U.S. Provisional Patent Application Ser. No. 63/783,735 titled “SYSTEM FOR RESPIRATION CONDITIONING,” filed Apr. 4, 2025, the entire contents of which is incorporated herein by reference for all purposes.

### FIELD OF THE INVENTION

Illustrative embodiments of the invention generally relate to respiratory therapy and, more particularly, illustrative embodiments relate to an implantable pulse generator used for respiratory therapy and conditioning.

### BACKGROUND

Neurostimulation provided by an implanted stimulation pulse generator can induce and modulate respiration. For example, stimulation of a phrenic nerve or vagus nerve may induce and modulate diaphragmatic contractions and may affect upper airway stability. As another example, stimulation of an upper airway nerve, such as a hypoglossal nerve or other nerve connected to muscles in the upper airway, may induce and modulate movement of upper airway muscles that affect respiration. In some patients, neurostimulation from an implanted stimulation pulse generator provides therapy for respiratory disorders related to sleep, such as central sleep apnea (CSA), obstructive sleep apnea (OSA), or combination apneas. CSA occurs when the brain fails to send proper signals to the diaphragm. This condition is different from OSA, where there is a physical blockage in the airway often due to collapsed or relaxed muscles in the upper airway. However, respiration occurs continuously, during waking and sleeping hours. Thus, the occurrence of respiratory disorders and the deleterious effects of these disorders on a patient's physiologic function are not limited to sleep related disorders.

### SUMMARY

The foregoing general description of the illustrative implementations and the following detailed description thereof are merely exemplary aspects of the teachings of this disclosure and are not restrictive.

In accordance with an example, an implantable pulse generator (IPG) is configured to provide respiratory conditioning for a patient. The IPG includes at least one processor configured to determine that a patient is in a non-sleep time period. The processor is also configured to enter a patient control operational mode as a function of determining that the patient is in the non-sleep time period. The patient control operational mode provides one or more respiration conditioning sessions. Each respective respiration conditioning session of the one or more respiration conditioning sessions corresponds to a pre-programmed conditioning stimulation profile specifying stimulation parameters for the respective respiration conditioning session. The processor is configured to receive a session activation signal from a patient mobile device to initiate the respective respiration conditioning session. In response to receiving the activation signal during the patient control operational mode, the processor controls delivery of electrical stimulation to a phrenic nerve of the patient according to the at least one pre-programmed conditioning stimulation profile to modulate patient respiration.

Implementations of such an IPG may include one or more of the following features. The at least one processor may be further configured to, among other things, determine that the patient is in a sleep time period. The process may enter a sleep operational mode having one or more sleep treatment sessions. Each of the one or more sleep treatment sessions may correspond to a pre-programmed sleep stimulation profile. The process may control delivery of the electrical stimulation to the phrenic nerve according to at least one pre-programmed sleep stimulation profile to modulate patient respiration during the sleep operational mode. Electrical stimulation may be delivered in an absence of any session activation signal from the patient mobile device. In some embodiments, the IPG may include at least one implantable stimulation lead that delivers the electrical stimulation to the phrenic nerve.

The at least one processor may be configured to select the pre-programmed conditioning stimulation profile from a library of pre-programmed conditioning stimulation profiles. Each of the pre-programmed conditioning stimulation profiles may have an associated patient breathing instruction configured to be displayed on a user interface of the patient mobile device.

Additionally, or alternatively, the conditioning stimulation profile may be selected by the user from the patient mobile device. In various embodiments, the IPG is configured to receive the selected conditioning stimulation profile from the patient mobile device. Thus, the IPG may be configured to receive an identifier for the selected conditioning stimulation profile from the patient mobile device. The IPG may also be configured to retrieve the selected conditioning stimulation profile from a library, stored at a memory of the IPG, having a plurality of conditioning stimulation profiles.

The at least one processor may be further configured to monitor a respiration signal received from at least one respiration sensor while in the patient control operational mode. The at least one processor may determine that patient compliance is below a performance threshold for a respiratory metric. The processor may also select a second conditioning stimulation profile from the plurality of conditioning stimulation profiles.

Delivery of the electrical stimulation to the phrenic nerve of the patient may be controlled according to the second conditioning stimulation profile. The processor may be configured to prematurely end the delivery of electrical stimulation according to the at least one pre-programmed conditioning stimulation profile.

The at least one processor may be configured to determine that the patient compliance is below a given threshold as a function of (1) subjective patient feedback data received from the patient mobile device, and/or (2) objective patient data received from at least one respiration sensor, while in the patient control operational mode. The subjective patient feedback data includes, among other things, an indication from the patient regarding pain from the electrical stimulation to the phrenic nerve. The indication may be received from the patient mobile device.

Among other things, objective patient data may include a respiration signal that relates to a breathing rate, tidal volume, and/or minute ventilation. The respiration signal may be obtained from the respiration sensor. Additionally, or alternatively, the objective patient data includes an oxygen saturation signal obtained from a pulse oximeter. The objective patient data may include a patient compliance score. The patient compliance score may include a comparison of the associated patient breathing instruction for the selected conditioning stimulation profile with corresponding patient respiratory data. The patient compliance score may be determined by at least one of the patient mobile device, an external computing device, and/or the at least one processor.

To conclude the at least one respiration conditioning session, the at least one processor may receive a deactivation signal. Upon receiving the deactivation signal, the processor may cause the stimulation circuitry to stop generating the electrical stimulation. In various embodiments, the non-sleep time period is a pre-determined time period. The at least one processer may communicate with a clock to determine when the IPG is in a sleep operational mode and when the IPG is a patient control operational mode.

The at least one processor may also be configured to receive a second session activation signal from a patient mobile device to initiate a second respiration conditioning session. The processor may control delivery of the electrical stimulation to the phrenic nerve of the patient according to a second conditioning stimulation profile in response to receiving the second activation signal during the patient control operational mode. The at least one processor may be further configured to receive multiple session activation signals from a patient mobile device, each corresponding to the initiation of a respective respiration conditioning session. In response to each activation signal received during the patient control operational mode, the processor may control the delivery of electrical stimulation to the patient's phrenic nerve according to a corresponding conditioning stimulation profile. This allows for repeated, session-specific stimulation delivery aligned with the respective conditioning profiles.

The process may be further configured to receive feedback regarding patient progress for the respiration conditioning session. The at least one pre-programmed sleep stimulation profile may be adjusted as a function of the feedback. In various embodiments, the at least one pre-programmed conditioning stimulation profile is configured to cause the at least one processor to deliver the electrical stimulation during the one or more respiration conditioning sessions.

A total time of the one or more respiration conditioning sessions may be less than a duration of the non-sleep time period. For example, each of the one or more respiration conditioning sessions may be 10-60 minutes in duration. All of the one or more respiration conditioning sessions may be different in duration. Alternatively, at least one of the one or more respiration conditioning sessions may be a same duration as at least one other of the one or more respiration conditioning sessions.

The at least one pre-programmed stimulation profile may be configured to cause the at least one processor to control the stimulation circuitry to change stimulation parameters according to a pre-determined pattern over a course of one or more respiration conditioning sessions duration for the at least one pre-programmed stimulation profile. The pre-determined pattern may include incremental increases of a stimulation current accompanied by incremental decreases of a stimulation rate. The pre-determined pattern may include a gradual increase of a length of a rising ramp of a stimulation current and a length of a falling ramp of the stimulation current. Among other things, the pre-determined pattern may include a gradual increase of a stimulation burst amplitude accompanied by a gradual decrease in stimulation rate. Furthermore, the pre-determined pattern may include a gradual increase of a stimulation energy with a constant stimulation rate.

The at least one processor may be configured to adjust one or more stimulation parameters over successive respiration conditioning sessions according to a conditioning profile. The conditioning profile may include (a) a gradual increase of stimulation energy over a first group of one or more respiration conditioning sessions, (b) a maintenance of the increased stimulation energy for a second group of one or more respiration conditioning sessions, and/or (c) a gradual reduction of a stimulation rate at the increased stimulation energy for a third group of one or more respiration conditioning sessions. The at least one processor may be configured to repeat the conditioning profile until the stimulation rate reaches a target stimulation rate. In various embodiments, the target stimulation rate may be 6-8 breaths per minute.

The at least one pre-programmed stimulation profile may be associated with a respiratory goal based on one or more respiratory parameters. For example, the respiratory goal may include a tidal volume goal. The one or more respiratory parameters may include patient tidal volume. As another example, the respiratory goal may include a minute ventilation goal, and the one or more respiratory parameters may include patient tidal volume and respiratory rate.

The respiration signal may be indicative of the one or more respiratory parameters of the patient. The at least one processor may be configured to track the one or more respiratory parameters of the patient over successive one or more respiration conditioning sessions. The process may determine a difference between the one or more respiratory parameters and the respiratory goal. The processor may store the one or more respiratory parameters, the difference, and an associated time stamp in a memory associated with the IPG. The processor may furthermore be configured to store one or more stimulation parameters associated with an achievement of the respiratory goal. For example, the stimulation parameters may be stored based on the difference between the one or more respiratory parameters and the respiratory goal being substantially zero. The at least one processor may be configured to implement the stored one or more stimulation parameters associated with an achievement of the respiratory goal for electrical stimulation provided during a pre-determined sleep time period for the patient.

The one or more respiratory parameters may include patient tidal volume. The at least one respiration sensor may include a transthoracic impedance sensor. The at least one processor may be configured to determine the tidal volume of the patient based on a transthoracic impedance signal from the transthoracic impedance sensor based on a pre-determined correlation between transthoracic impedance and tidal volume for the patient. The at least one processor may be configured to monitor a signal from a fluid load sensor and calibrate the value of the transthoracic impedance signal to the tidal volume of the patient based on the fluid load of the patient.

The at least one processor may be configured to implement multiple different pre-programmed stimulation profiles. The therapy controller may be configured to provide the delivery of the electrical stimulation to the phrenic nerve at an intensity below an intensity level that activates the diaphragm, such that the electrical stimulation provides a reminder to breath without functionally causing a diaphragm contraction. The IPG may be configured to communicatively couple to a patient-specific external computing device via the communications interface.

The at least one processor may be further configured to receive the activation signal from the patient-specific external computing device responsive to a first user input to the patient-specific external computing device to initiate the electrical stimulation. The at least one processor may be further configured to discontinue the electrical stimulation responsive to reception, via the communications interface, of a second user input to the patient-specific external computing device to stop the electrical stimulation.

In accordance with another embodiment, a system for providing respiratory conditioning for a patient includes an implantable pulse generator (IPG) configured to provide electrical stimulation to a phrenic nerve of the patient. The electrical stimulation is provided via at least one stimulation lead in accordance with a selected stimulation profile. The system includes an electronic device having a user interface configured to display a patient breathing instruction corresponding to the selected stimulation profile.

The system may also include a data storage system having a stimulation profile library with a plurality of stimulation profiles. Each of the plurality of stimulation profiles may have an associated patient breathing instruction. The at least one processor may be configured to control delivery of the electrical stimulation to the phrenic nerve of the patient according to the selected stimulation profile. The at least one processor may be configured to determine that a patient is in a non-sleep time period. The at least one processor may be configured to enter the non-sleep time period therapy mode based at least in part on a pre-programmed start time-of-day and a pre-programmed end time-of-day.

The system may include stimulation circuitry configured to generate an electrical stimulation for a first respiration conditioning session in accordance with a first conditioning stimulation profile during the non-sleep time period. The system may include at least one processor configured to receive an input for an activation signal for the first respiration conditioning session. The system may also include at least one processor configured to provide a first corresponding breathing instruction to the patient during the first respiration conditioning session for the first conditioning stimulation profile.

The system may include at least one processor configured to receive conditioning feedback from the patient regarding the first respiration conditioning session. The conditioning feedback may include an indication regarding patient comfort during the stimulating, data representative of patient respiration, and/or data representing patient compliance with the first corresponding breathing instruction.

The at least one processor may be configured to control stimulation of the patient for a second respiration conditioning session, during the non-sleep time period, in accordance with a second conditioning stimulation profile that is a function of the conditioning feedback. A second corresponding breathing instruction may be provided to the patient, during the second respiration conditioning session, for the second conditioning stimulation profile.

The at least one processor may be configured to, among other things, determine that the patient is in a sleep time period. The processor may control stimulation of the patient for a first sleep stimulation treatment to modulate patient respiration, during the sleep time period, in accordance with a first sleep stimulation profile. The at least one processor may be further configured to control stimulation of the patient for a second sleep stimulation treatment, occurring after the first respiration conditioning session, in accordance with a second sleep stimulation profile that is a function of the conditioning feedback. The second sleep stimulation profile may have a different stimulation energy than the first sleep stimulation profile.

The processor may be configured to receive an activation signal to enter to initiate at least one respiration conditioning session from a user input into the user interface. Additionally, the system also confirms that the time is in an awake time period, and that a motion and pitch of the user indicates that they are awake. Alternatively, the system may use time, motion and pitch of the user to enter the sleep apnea mode.

In various embodiments, the system may receive a user input to terminate a given respiration conditioning session. The system may further provide an instruction to terminate the given respiration conditioning session prior to delivery of all of the electrical stimulation in a corresponding conditioning stimulation profile. The system may also receive a user input to lower the electrical stimulation in a given respiration conditioning session. The processor may provide an instruction to lower the intensity of the given respiration conditioning session prior to delivery of all of the electrical stimulation in a corresponding conditioning stimulation profile.

The system may include a respiration sensor configured to provide patient respiration feedback indicating that the patient compliance with given breathing instructions for a given respiration conditioning session is below a threshold. The at least one processor may be configured to adjust the given respiration conditioning session prior to delivery of all of the electrical stimulation in a corresponding conditioning stimulation profile.

The at least one processor may be further configured to control the stimulation circuitry based at least in part on signals from at least one of an electromyography sensor, a cardiac cycle sensor, an air flow sensor, an internal pressure sensor, a pulse oximeter, a chest belt, or a peripheral arterial tone sensor.

In various embodiments, each of the pre-programmed stimulation profiles has an associated patient breathing instruction. The associated patient breathing instruction provides breathing instructions to the patient. For example, the breathing instruction may be provided to the patient on a graphical user display of a smartphone.

In various embodiments, the system is configured to monitor a signal from at least one patient motion sensor indicative of one or more of patient movement or patient position. The system determine one or more of motion or position parameters based on the signal. The system then enters the non-sleep time period therapy mode based at least in part on the one or more of motion or position parameters. The at least one patient motion sensor includes at least one of an implanted accelerometer or an accelerometer coupled to an exterior surface of the patient.

The least one processor may be configured to enter a sleep apnea therapy operational mode during a pre-determined sleep period for the patient identified at least in part based on the pre-programmed start time-of-day and the pre-programmed end time-of-day. Furthermore, at least one processor is further configured to control the stimulation circuitry to deliver electrical stimulation during the pre-determined sleep time period at a higher energy than during the pre-determined non-sleep time period. The at least one processor may be within a housing of the electronic device. Additionally, or alternatively, the at least one processor may be within a housing of the implantable pulse generator.

The IPG may further include at least one battery. The at least one battery may include one or more of a rechargeable battery or a non-rechargeable battery. The at least one battery may include the rechargeable battery, and the IPG may include magnetic inductive charging circuitry configured to provide a recharging current to the rechargeable battery.

The IPG may include one or more receptacles configured to couple to at least one implantable stimulation lead configured to provide electrical stimulation to an upper airway nerve. The IPG may be configured to provide one or more of bilateral phrenic nerve stimulation or unilateral phrenic nerve stimulation. The one or more receptacles may be configured to couple to the at least one implantable stimulation lead comprising a cuff electrode or a transvenous electrode. The one or more receptacles may be configured to couple to the at least one implantable stimulation lead comprising the at least one respiration sensor. A header may include at least one receptacle configured to couple to a at least one implantable sensing lead comprising the at least one respiration sensor.

Various embodiments, the system includes a wireless communications interface is configured to communicate via one or more of telemetry or a Bluetooth® communications protocol. The wireless communications interface may be configured to communicate with at least one patient-specific external computing device. The electronic device may include one or more of a smartphone, a smartwatch, a laptop, or a wearable computing device. The at least one respiration sensor comprises a transthoracic impedance sensor.

In accordance with yet another embodiment a patient electronic device is configured for use with an implantable pulse generator. The implantable pulse generator is configured to provide electrical stimulation to a phrenic nerve of the patient via at least one stimulation lead in accordance with a selected stimulation profile. The patient electronic device includes at least one processor configured to determine that the implantable pulse generator is in a patient control mode. The processor is also configured to generate an associated patient breathing instruction for a first respiration conditioning session having the selected stimulation profile. The patient electronic device also includes a user interface configured to receive a user input to send a session activation signal to the implantable pulse generator to begin the first respiration conditioning session having the selected stimulation profile. The user interface provides the associated patient breathing instruction for the selected stimulation profile. The associated patient breathing instruction may be temporally aligned with the selected stimulation profile.

The user interface may be a display. The associated patient breathing instruction may thus be provided to the patient visually. The device may include a speaker. The associated patient breathing instruction may be provided audibly. The device may also include a tactile feedback module. The associated patient instruction may be provided to the patient tactilely. For example, the device may vibrate at a set intensity or frequency to indicate various respiratory commands to the patient.

Among other things, the user interface is configured to receive subjective patient feedback. The subjective patient feedback may include an indication from the patient regarding pain from an electrical stimulation to the phrenic nerve according to the selected stimulation profile.

The user interface may be configured to receive conditioning feedback from the patient regarding the first respiration conditioning session. The conditioning feedback may include an indication regarding patient comfort during the stimulating, data representative of patient respiration, and/or data representing patient compliance with the first corresponding breathing instruction.

In accordance with an example, an implantable pulse generator (IPG) is configured to provide respiratory therapy to treat atrial fibrillation for a patient. The IPG includes at least one processor configured to determine that the patient is in a sleep time period, enter a sleep operational mode, the sleep operational mode having one or more sleep treatment sessions, each of the one or more sleep treatment sessions corresponding to a pre-programmed sleep stimulation profile, and control delivery of electrical stimulation to a phrenic nerve according to at least one pre-programmed sleep stimulation profile to modulate patient respiration during the sleep operational mode. The at least one processor is configured to enter the sleep operational mode based on a patient therapy mode indicator comprising an atrial fibrillation therapy indicator.

Implementations of such an IPG may include one or more of the following features. The at least one processor may be configured to enter the sleep operational mode based on a patient therapy mode indicator comprising a sleep apnea therapy indicator or a combination sleep apnea-atrial fibrillation indicator. The at least one processor may be configured to allow a selection of the patient therapy mode indicator from a group of options comprising a sleep apnea therapy indicator, the atrial fibrillation therapy indicator, and a combination sleep apnea-atrial fibrillation indicator. The pre-programmed sleep stimulation profile may be a first profile targeting atrial fibrillation therapy and may include different stimulation parameters than a second profile targeting sleep apnea therapy. The second profile may be associated with a different IPG than the first profile. A stimulation rate of the first profile may be less than a stimulation rate of the second profile. A stimulation amplitude of the first profile may be greater than a stimulation amplitude of the second profile. The pre-programmed sleep stimulation profile may be a first profile targeting atrial fibrillation therapy and may include equivalent stimulation parameters to a second profile targeting sleep apnea therapy. The second profile may be associated with a different IPG than the first profile. The at least one pre-programmed sleep stimulation profile may specify a number of discrete stimulation periods provided intermittently for the sleep time period such that the sleep time period includes a plurality of non-stimulation time periods substantially longer than an inhalation-exhalation cycle. The at least one processor may be further configured to determine that the patient is in a sleep time period, access a cardiac status indicator, and enter the sleep operational mode based on the cardiac status indicator. The at least one processor may be further configured to bypass the sleep operational mode based on the cardiac status indicator indicating an improvement in AF metrics, and enter the sleep operational mode based on the cardiac status indicator indicating a deterioration in the AF metrics. The IPG may include at least one cardiac sensor and be configured to monitor signals from the at least one cardiac sensor and determine the cardiac status indicator based on the monitored signals. The signals from the at least one cardiac sensor may include one or more of heart rate signals or electrocardiogram signals. The at least one processor may be configured to select the pre-programmed sleep stimulation profile from a library of pre-programmed sleep stimulation profiles based on the patient therapy mode indicator. The IPG may include least one implantable stimulation lead configured to deliver the electrical stimulation to the phrenic nerve. The at least one processor may be configured to select the pre-programmed sleep stimulation profile from a library of pre-programmed sleep stimulation profiles. The at least one processor may be configured to select the pre-programmed sleep stimulation profile from a library of sleep stimulation profiles based on a patient therapy mode indicator comprising a sleep apnea therapy indicator, an atrial fibrillation therapy indicator, or a combination sleep apnea-atrial fibrillation indicator. The at least one processor may be configured to determine that the patient is in the sleep time period based at least in part on communication with a clock and a pre-determined sleep time period. The at least one pre-programmed sleep stimulation profile may be configured to cause the at least one processor to deliver the one or more sleep treatment sessions intermittently such that a time between at least two sleep treatment sessions is substantially longer than an inhalation-exhalation cycle for a patient. Each of the one or more sleep treatment sessions may be 5-60 minutes in duration. A duration of at least one sleep treatment session may span a time from a determination that the patient is in the sleep time period to a determination that the patient is aroused based on one or more of a time and a motion detection. The at least one pre-programmed sleep stimulation profile may be configured to provide a stimulation rate of 10-18 bpm and is independent of a patient's intrinsic breathing rate along with a stimulation amplitude of 4-8 mA. The at least one pre-programmed sleep stimulation profile may provide a duty cycle of 5-10% or 5-15%. The at least one processor may be further configured to determine that a patient is in a non-sleep time period, enter a patient control operational mode as a function of determining that the patient is in the non-sleep time period, the patient control operational mode providing one or more respiration conditioning sessions, each respective respiration conditioning session of the one or more respiration conditioning sessions corresponding to at least one pre-programmed conditioning stimulation profile specifying stimulation parameters for a respective respiration conditioning session, receive a session activation signal from a patient mobile device to initiate the respective respiration conditioning session, and control delivery of electrical stimulation to a phrenic nerve of the patient according to the at least one pre-programmed conditioning stimulation profile to modulate patient respiration in response to receiving the session activation signal during the patient control operational mode. Each pre-programmed conditioning stimulation profile may have an associated patient breathing instruction configured to be displayed on a user interface of the patient mobile device. The at least one pre-programmed conditioning stimulation profile may be selected by a user from the patient mobile device. The IPG may be configured to receive the at least one pre-programmed conditioning stimulation profile from the patient mobile device. The IPG may be configured to receive an identifier for the at least one pre-programmed conditioning stimulation profile from the patient mobile device, and retrieve the at least one pre-programmed conditioning stimulation profile from a library stored at a memory of the IPG having a plurality of conditioning stimulation profiles. The at least one processor may be further configured to monitor a respiration signal received from at least one respiration sensor while in the patient control operational mode. The at least one processor may be configured to determine that patient compliance is below a performance threshold for a respiratory metric, select a second conditioning stimulation profile from a plurality of stored conditioning stimulation profiles, and control delivery of the electrical stimulation to the phrenic nerve of the patient according to the second conditioning stimulation profile. The at least one processor may be further configured to prematurely end the delivery of electrical stimulation for the at least one pre-programmed conditioning stimulation profile. The at least one processor may be configured to determine that the patient compliance is below a given threshold as a function of (1) subjective patient feedback data received from the patient mobile device, and/or (2) objective patient data received from at least one respiration sensor, while in the patient control operational mode. The subjective patient feedback data may include an indication from the patient regarding pain from the electrical stimulation to the phrenic nerve, the indication being received from the patient mobile device. The objective patient data may include a respiration signal that relates to a breathing rate, tidal volume, and/or minute ventilation, obtained from a respiration sensor. The objective patient data may include an oxygen saturation signal obtained from a pulse oximeter. The objective patient data may include a patient compliance score, the patient compliance score including a comparison of patient breathing instructions associated with the at least one pre-programmed conditioning stimulation profile with corresponding patient respiratory data. The patient compliance score may be determined by at least one of the patient mobile device, an external computing device, and/or the at least one processor. The at least one processor may be configured to receive a deactivation signal to conclude at least one respiration conditioning session, the at least one processor further configured to cause IPG stimulation circuitry to stop generating the electrical stimulation. The at least one processor may be configured to receive a second session activation signal from a patient mobile device to initiate a second respiration conditioning session, and control delivery of the electrical stimulation to the phrenic nerve of the patient according to a second conditioning stimulation profile in response to receiving the second session activation signal during the patient control operational mode. The at least one pre-programmed conditioning stimulation profile may be configured to cause the at least one processor to deliver the electrical stimulation during the one or more respiration conditioning sessions, wherein a total time of the one or more respiration conditioning sessions may be less than a duration of the non-sleep time period. Each of the one or more respiration conditioning sessions may be 10-60 minutes in duration. The IPG may be configured to communicatively couple to a patient-specific external computing device via a communications interface. The at least one processor may be further configured to receive an activation signal from the patient-specific external computing device responsive to a first user input to the patient-specific external computing device to initiate the electrical stimulation. The at least one processor may be further configured to prematurely end the electrical stimulation responsive to reception, via the communications interface, of a second user input to the patient-specific external computing device to stop the electrical stimulation.

In accordance with an example, a method of providing respiratory therapy to treat atrial fibrillation for a patient may include determining that the patient is in a sleep time period based at least in part on communication with a clock and a pre-determined sleep time period, entering a sleep operational mode, the sleep operational mode having one or more sleep treatment sessions, each of the one or more sleep treatment sessions corresponding to a pre-programmed sleep stimulation profile, and controlling, by an implantable pulse generator (IPG), delivery of electrical stimulation to a phrenic nerve via at least one implantable stimulation lead according to at least one pre-programmed sleep stimulation profile to modulate patient respiration during the sleep operational mode. The IPG may be configured to enter the sleep operational mode based on a patient therapy mode indicator comprising an atrial fibrillation therapy indicator.

Implementations of such a method may include one or more of the following features. The method may include allowing a selection of the patient therapy mode indicator from a group of options comprising a sleep apnea therapy indicator, the atrial fibrillation therapy indicator, and a combination sleep apnea-atrial fibrillation indicator. The pre-programmed sleep stimulation profile may be a first profile targeting atrial fibrillation therapy and include different stimulation parameters than a second profile targeting sleep apnea therapy or stimulation parameters substantially equivalent to the second profile targeting sleep apnea therapy. The method may include determining that the patient is in a sleep time period, accessing a cardiac status indicator, bypassing the sleep operational mode based on the cardiac status indicator indicating an improvement in AF metrics, and entering the sleep operational mode based on the cardiac status indicator indicating a deterioration in the AF metrics. The at least one pre-programmed sleep stimulation profile may be configured to provide a stimulation rate of 10-18 bpm and may be independent of a patient's intrinsic breathing rate along with a stimulation amplitude of 4-8 mA and wherein the at least one pre-programmed sleep stimulation profile may provide a duty cycle of 5-10% or 5-15%. The method may further include monitoring signals from at least one cardiac sensor, and determining at least one cardiac status indicator based on the monitored signals, wherein the signals from the at least one cardiac sensor comprise one or more of heart rate signals or electrocardiogram signals. The method may further include communicatively coupling, by the IPG, to a patient-specific external computing device via a communications interface, and receiving an activation signal from the patient-specific external computing device responsive to a first user input to the patient-specific external computing device to initiate the electrical stimulation.

Illustrative embodiments may be implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various exemplary embodiments from the following “Description of Illustrative Embodiments,” discussed with reference to the drawings summarized immediately below.

FIG. 1 schematically shows a system for respiratory conditioning an awake patient in accordance with illustrative embodiments.

FIG. 2A-1 shows a process of determining a sleep therapy or non-sleep patient control operational mode of the system of FIG. 1 in accordance with illustrative embodiments.

FIG. 2A-2 shows a process of determining a patient therapy mode in accordance with illustrative embodiments.

FIG. 2A-3 shows a process of determining an atrial fibrillation operational mode for sleep time and non-sleep patient control operational modes of the system of FIG. 1 in accordance with illustrative embodiments.

FIG. 2A-4 shows a process of determining a combination of atrial fibrillation and sleep apnea operational mode for sleep time or non-sleep patient control operational mode of the system of FIG. 1 in accordance with illustrative embodiments.

FIG. 2B shows a detailed process of the patient control mode of the system of FIG. 1 in accordance with illustrative embodiments.

FIG. 2C shows a detailed process of the patient control mode of the system of FIG. 1 in accordance with illustrative embodiments.

FIG. 3 schematically shows a pre-determined sleep time period and a pre-determined non-sleep time period in accordance with illustrative embodiments.

FIGS. 4-7B schematically show a user interface of the patient control mode in accordance with illustrative embodiments.

FIG. 8A schematically shows an example or sleep stimulation profile progress without feedback, in accordance with illustrative embodiments.

FIG. 8B schematically shows an example of adjusting sleep stimulation profiles using feedback from respiratory conditioning sessions, in accordance with illustrative embodiments.

FIG. 9 schematically shows an example of feedback from one or more respiratory conditioning sessions being used to adjust stimulation profiles for one or more subsequent respiratory conditioning sessions in accordance with illustrative embodiments.

FIG. 10 illustrates details of an electrical stimulation profile in accordance with illustrative embodiments.

FIG. 11A schematically shows details of a neurostimulation system in accordance with illustrative embodiments.

FIG. 11B schematically shows details of a processor and memory of the IPG in accordance with illustrative embodiments.

FIG. 11C schematically shows detailed on the implantable pulse generator of FIG. 1.

FIGS. 12A-12F show swim-lane diagrams depicting operations of the processor in accordance with illustrative embodiments.

FIG. 13 schematically shows aspects of asynchronous and synchronous sub-modes of operation for the IPG in accordance with illustrative embodiments.

### DESCRIPTION OF ILLUSTRATIVE EMBODIMENTS

In illustrative embodiments, a system includes an implantable neurostimulation pulse generator to provide respiratory conditioning for an awake patient. The system includes a patient control mode with a user interface that allows patients to select a respiratory conditioning regimen with physician supervision and guides the awake patient with breathing instructions as part of the selected respiratory conditioning regimen. The pulse generator delivers controlled electrical impulses to one or more nerves related to respiratory function in accordance with the selected respiratory conditioning regimen. The respiratory conditioning regimen may include one or more neurostimulation sessions during the waking hours for a patient when the patient is exerting conscious control over breathing. The pulse generator may also provide neurostimulation during the sleeping hours for the patient when the patient is not exerting conscious control over breathing. In this case, the neurostimulation may be provided according to pre-programmed stimulation profiles directed at eliminating sleep apneas.

In the case of phrenic nerve stimulation, the neurostimulation pulse generator may only stimulate one of the two phrenic nerves (e.g., the right phrenic nerve or the left phrenic nerve). With this unilateral stimulation, the patient retains control over breathing and can override the stimulation. In other words, the patient can fight or resist the breathing rhythm generated by the neurostimulation and maintain a different, and in some cases less beneficial breathing rhythm. This less beneficial breathing rhythm may include apneas, hypopneas, hyperpneas, and other patterns of disordered breathing.

One difficulty in providing disordered breathing therapy during the sleeping hours is patient acclimation to the neurostimulated breathing. Anxiety and discomfort with the neurostimulation may cause the patient to fight the stimulation and may cause waking or fitfulness. Such waking or fitfulness runs counter to the goal of sleeping therapy to improve sleep quality for the patient. For example, in some cases, acclimation may occur over weeks or months thus delaying the full benefit of the sleep disordered breathing therapy. Respiratory conditioning during waking hours may accelerate acclimation of a patient to the neurostimulation therapy provided during sleeping hours. As additional benefits, the respiratory conditioning during waking hours may train a patient to take deeper and slower breaths providing an overall benefit to nerve tone, cardiac health, mental health, and/or muscle strength. Deeper and slower breaths may decrease adrenaline levels and improve parasympathetic performance.

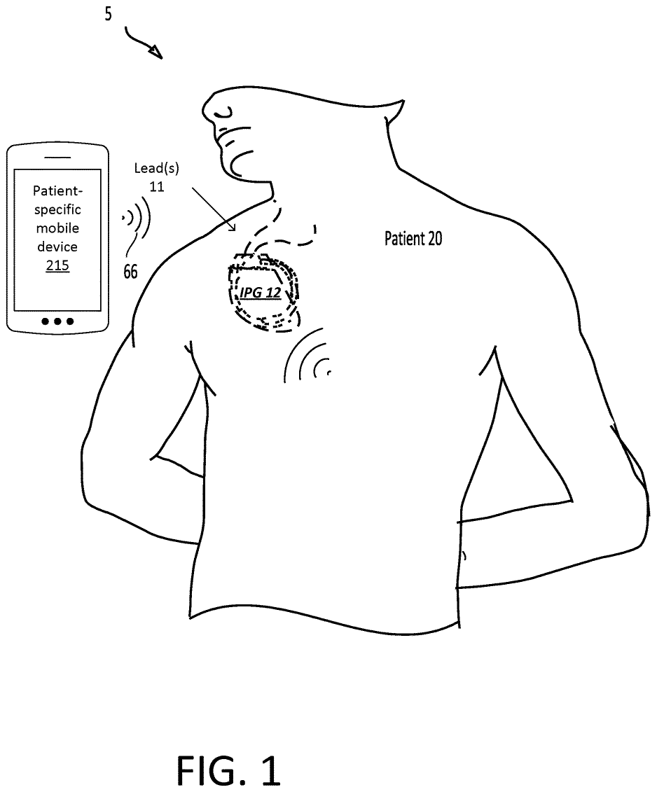

The respiratory conditioning regimens described herein are provided to an awake patient, and therefore, the neurostimulation device may require additional capabilities as compared with a neurostimulation device that is designed for use during sleeping hours. These capabilities may include a capability of interacting with and/or being controlled by an awake patient throughout provision of neurostimulation, as opposed to interaction and/or control at just the beginning (i.e., right before a patient goes to sleep) and/or just the end (i.e., right after a patient awakens) of a provision of stimulation. These capabilities may further include the capability to provide effect stimulation but at energies compatible with an awake patient as an awake patient may not tolerate the same stimulation energy as a sleeping patient. In some cases, the daytime stimulation may merely prompt the patient to breathe without actually inducing a diaphragmatic contraction. Such prompting may still provide conditioning benefits. A further capability may be to provide stimulation in a manner that does not interfere with daytime activities such as eating, talking, or physical exertion. These considerations are irrelevant for therapy during sleeping hours. Details of illustrative embodiments are discussed below. FIG. 1 schematically shows a system 5 for respiratory conditioning an awake patient 20 in accordance with illustrative embodiments described herein. The system 5 may also be used to treat sleep disordered-breathing in a patient.

Respiratory conditioning for an awake patient typically involves daytime training sessions (or sessions during waking hours) to improve and/or expedite patient acclimation to neurostimulation, improve respiratory rate control, and/or establish muscle memory. These improvements may be leveraged to improve efficacy and acclimation to sleep disordered breathing therapy. The process may include gradually adjusting stimulation parameters (e.g., intensity, frequency, pulse width) to promote deeper, slower breathing and progressively acclimating the patient to the neurostimulation of respiratory nerves.

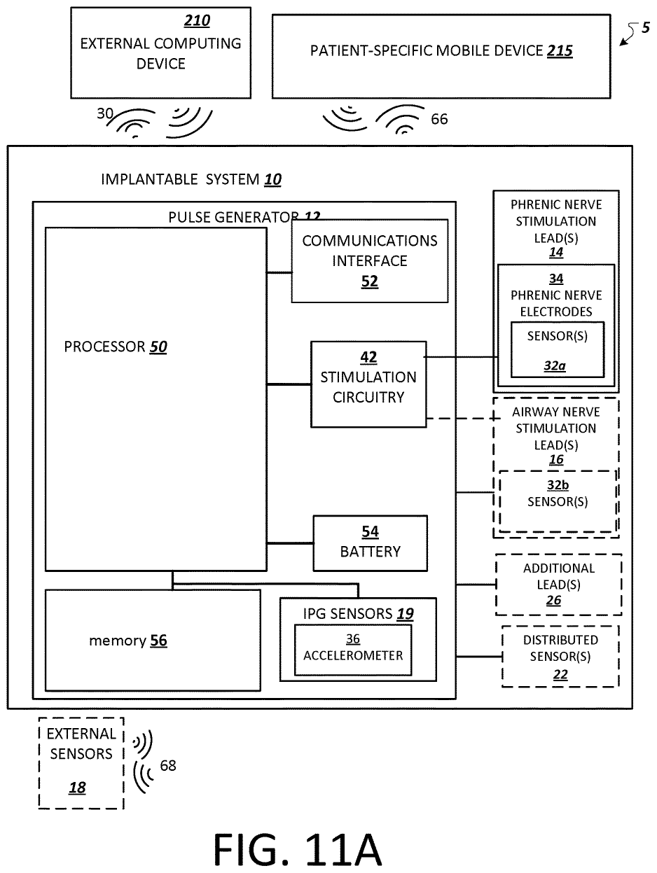

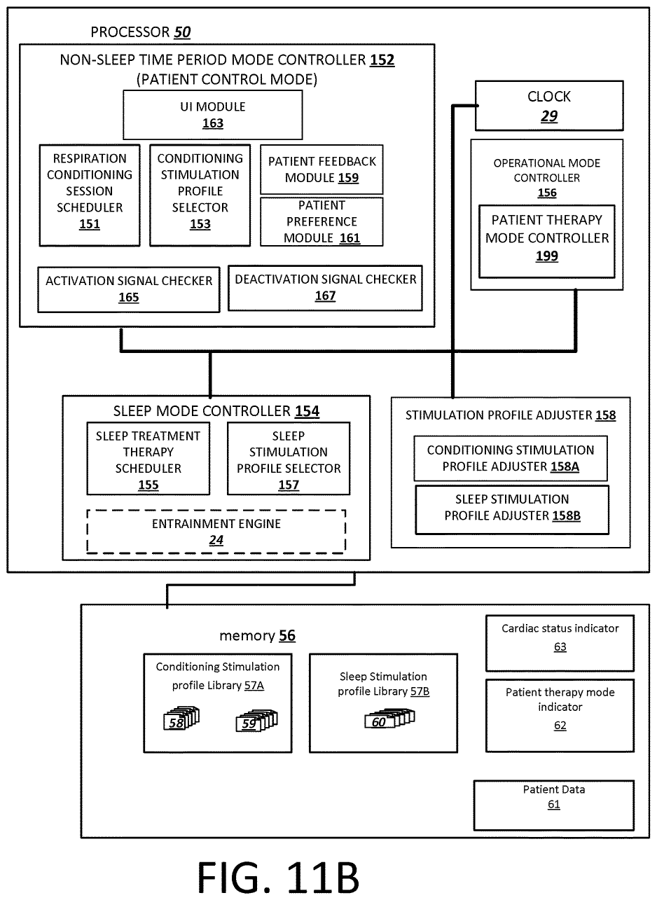

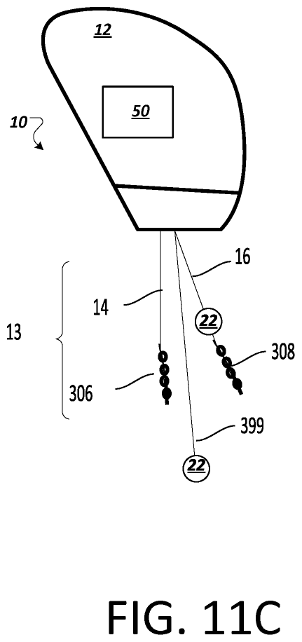

To that end, the system 5 includes one or more implantable components (also referred to as an implantable system 10) and external components that include a patient-specific mobile device 215. Additional external components are discussed in regard to FIGS. 11A-11C. Patient control input may include patient control via touch or voice feedback to a patient-specific computing device 215 (for example, a smartphone, watch, a handheld or worn remote controller, etc.). Such feedback may be used to start, stop, and/or reduce or increase the intensity of therapy stimulation (e.g., respiratory conditioning). Patient control input may also include patient control via touch feedback to the implanted system 10, such as a tap on the chest above the implanted system 10 to reduce or increase the intensity of stimulation.

The implantable components 10 include an implantable pulse generator 12 (IPG 12), which may be positioned under the skin of the patient 20 (e.g., in the upper chest area). The IPG 12 may include a battery or may be externally powered (e.g., wireless powered). Accordingly, at least a portion of the respiratory conditioning system 5 may be considered to be implantable. The IPG 12 is configured to couple to, and may be implanted with, one or more leads 11. The leads 11 may be thin conductors, such as wires, coupled to and/or including one or more electrodes. The one or more leads may be used to stimulate, via at least one electrode, one or more target nerves of the patient. For example, the leads may be used to stimulate a nerve in the chest, diaphragmatic nerve, such as the right and/or left phrenic nerve and/or an upper airway nerve or the patient. The upper airway nerve may include such as the hypoglossal nerve or the ansa cervicalis. The IPG 12 may generate the stimulation bursts used to stimulate the one or more nerves of the patient and provide the stimulation bursts to the one or more leads 11. Accordingly, the system 10 may be used to provide electrical stimulation of a target nerve via the one or more leads 11.

In some embodiments, the IPG 12 may be single channel and stimulate the phrenic nerve. In some other embodiments, the IPG 12 may be dual channel, and stimulate the phrenic nerve and the upper airway nerve. Stimulation may be unilateral (e.g., stimulate the right phrenic nerve or the left phrenic nerve) or bi-lateral (e.g., stimulate both phrenic nerves). Some embodiments may include a dual channel device that stimulates the phrenic nerve only during the day, but may stimulate the phrenic and/or upper airway nerve at night. Respiratory conditioning in an awake and conscious patient via stimulation of the diaphragm may only require stimulation of one or two phrenic nerves. However, treatment of sleep apnea in the sleeping patient may require stimulation of one or two phrenic nerves for CSA, stimulation of one or more upper airway nerves for OSA, or a combination of phrenic and upper airway nerves for combination apneas. Alternatively, the dual channel device may stimulate both the phrenic nerve and/or the upper airway nerve in both operational modes.

The system 5 may be used to provide electrical stimulation of a target nerve to support respiratory conditioning of an awake patient 20 (also referred to as respiration conditioning). Additionally, or alternatively, the system 5 may restore normal breathing patterns in patients with sleep disordered breathing. As described in further detail in regard to FIGS. 11A-11C, the system 5 may include and/or be coupled to one or more sensors to sense information about the patient 20 and/or treatment provided by the system 10, and evaluate and/or modify the treatment based on the information.

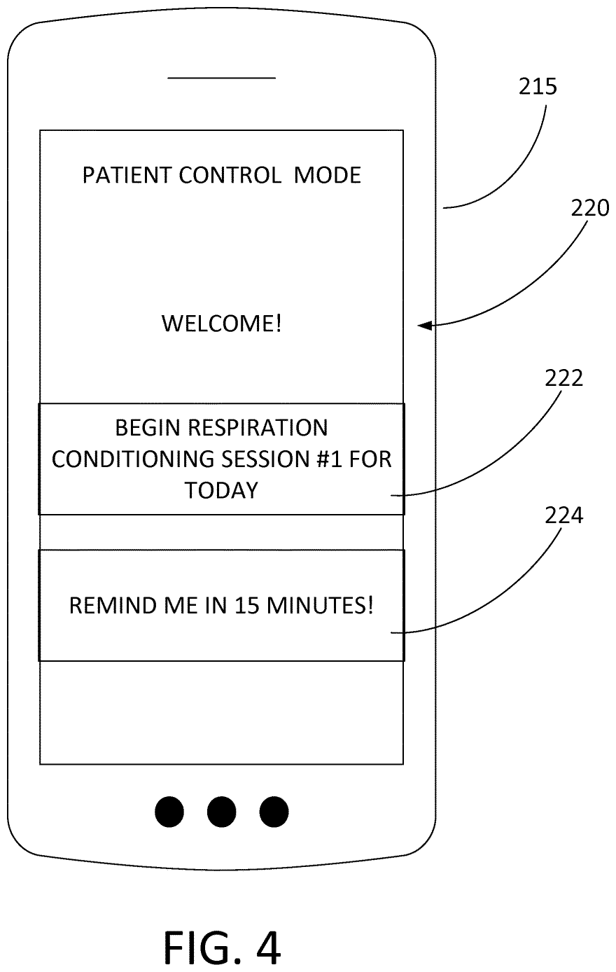

The system 5 may further include a patient-specific mobile device 215 configured to communicate via a wireless communication link 66 with the IPG 12. In various examples, the patient-specific mobile device 215 may be one or more of a smartphone, a smartwatch, another wearable computing device, or a laptop. For example, the wireless communication link 66 can be a telemetry communication connection and/or a Bluetooth® wireless communication connection between suitable circuitry in each of computing device 215 and the implanted (or implantable) pulse generator (IPG) 12 of system 10. In various embodiments, the patient-specific mobile device 215 includes a user interface, such as a display, through which instructions may be provided to the patient. In particular, the instructions are breathing instructions for a given respiratory conditioning regime. Furthermore, the user interface may provide selectable options, configured to receive an input from the user (e.g., begin respiration conditioning session). As best shown in FIGS. 4-7C, the patient-specific mobile device 215 provides selectable options and patient instructions via the user interface. In some embodiments, the instructions may additionally or alternatively be provided audibly (e.g., via a speaker of the patient-specific mobile device 215) and/or tactilely (e.g., via a haptic device or other tactile feedback module of the patient-specific mobile device 215).

In some embodiments, the patient-specific mobile device 215 may comprise a wand-type controller that includes physical buttons for turning therapy on and off, adjusting stimulation intensity, and switching between pre-programmed profiles. The patient-specific mobile device 215 may have a simple display or light indicators to confirm activation, status, or adjustments. Additionally, or alternatively, the patient-specific mobile device 215 may include a smartphone (app or similar device) that may provide more advanced functionality, such as real-time visual feedback on breathing patterns, options to choose stimulation profiles, or notifications about session progress.

In various examples, the system 5 includes one or processors configured to process information and control operation of the system 5, such as by delivering and modifying the treatment. Accordingly, when the system 5 or IPG 12 are described as determining information or parameters, executing operations such as comparing values to thresholds, modifying parameters for electrical stimulation, sensing or obtaining parameters or information, and so forth, it is to be appreciated that the one or more controllers and/or processors of the system 5 or IPG 12 may be performing these operations.

For example, 5 the IPG 12 may include at least one embedded controller 50 that includes at least one or more processors, at least one memory, at least one power source (for example, a battery), and power adjusting circuitry. The one or more processors may be configured to control the power adjusting circuitry in order to modulate the electrical stimulation as discussed in further detail in regard to FIG. 11A. The IPG 12 may therefore act as an electrical-pulse generator at least because the power adjusting circuitry may modulate the electrical stimulation. In various embodiments, the processor 50 may be an embedded processor within the IPG 12, and therefore implanted in the patient, in contrast to the processor of an external computing device (e.g., the patient-specific mobile device 215 or the computing device 210 as shown in FIG. 11A), which is external to the patient.

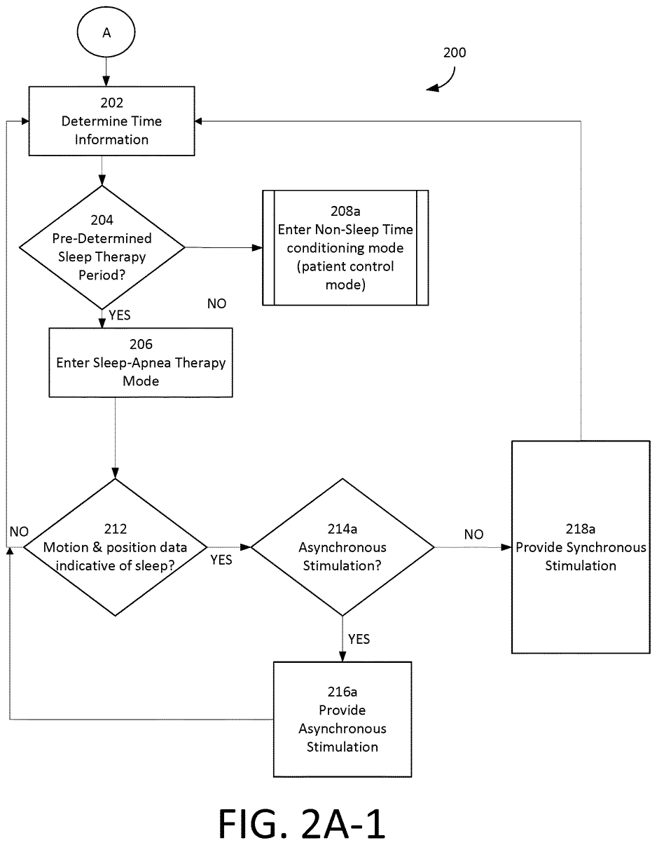

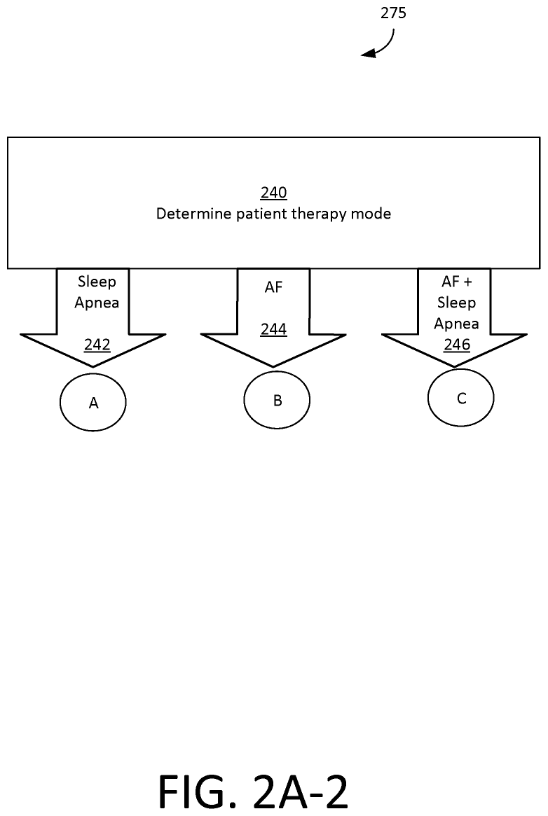

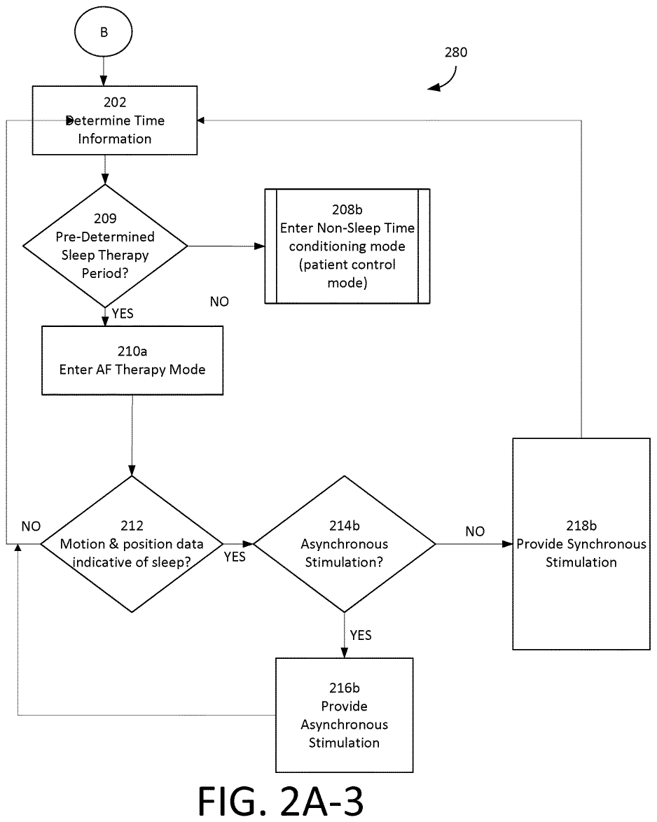

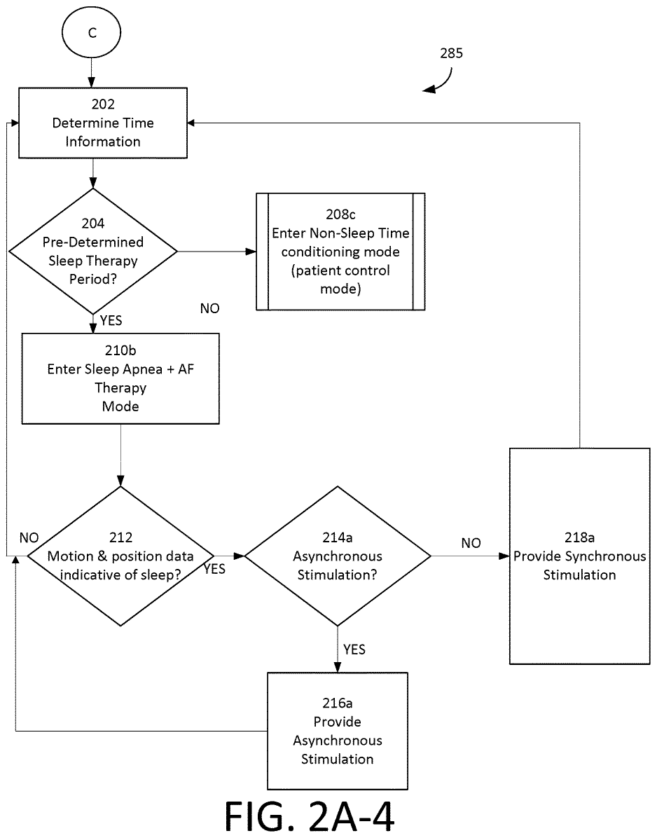

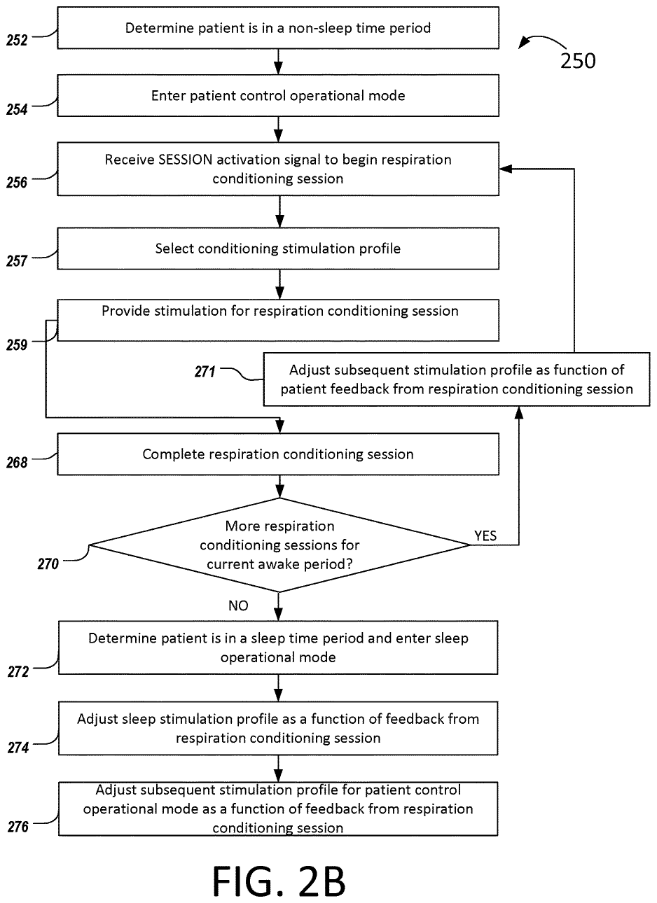

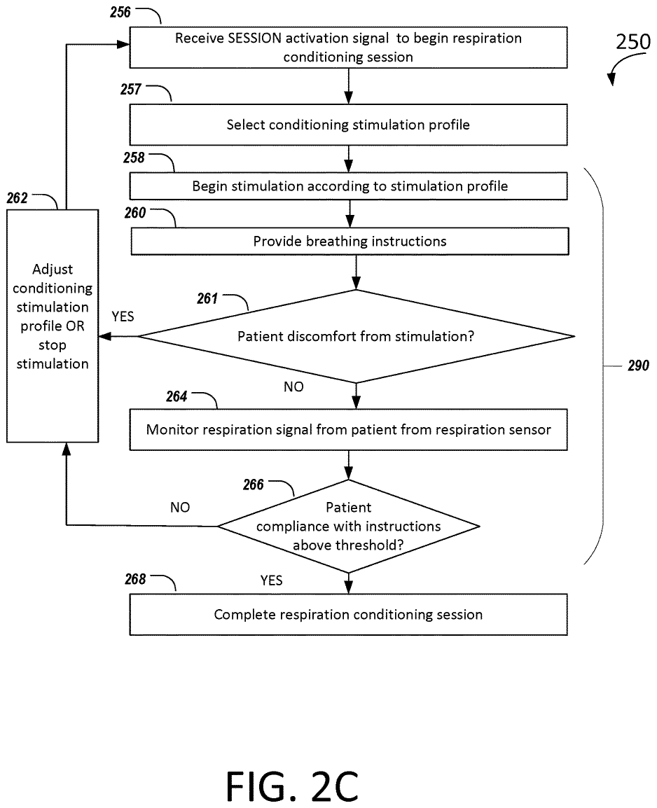

FIG. 2A-1 shows a process 200 of determining an operational mode of the system of FIG. 1 in accordance with illustrative embodiments described herein. For example, the processor 50 may perform steps of the process 200. The operational mode may be one of a sleep-apnea therapy mode or a patient control mode for respiratory conditioning. The patient control mode for respiratory conditioning may provide therapeutic benefits for sleep apnea. A device designed for sleep-apnea therapy without any daytime, or awake patient, therapy, may only provide one mode of operation, a sleep time therapy mode. Such a single mode device may determine if the patient is or is not asleep, either through patient input or based on various sensor data. If the patient is asleep, then the single mode device may enable provision of neurostimulation. If the patient is not asleep, then the single mode device does not enable provision of any neurostimulation. Further, such a single mode device may only provide for neurostimulation appropriate for a sleeping patient in regard to stimulation energy that may be intolerable for an awake patient and/or stimulation patterns that may not account for daytime activities and/or may not allow for or require patient interaction during the provision of stimulation. Thus even if such a single mode device were activated during a daytime hour, the device would lack the capability to provide appropriate neurostimulation for an awake patient. In contrast, the device as described herein provides for two distinct operational modes where the non-sleep time conditioning mode provides appropriate therapy for an awake patient, stimulation patterns that account for daytime activities and/or allows for or requires patient interaction during the provision of stimulation.

The method shown in FIG. 2A-1 may include additional other steps and some of the steps may be performed in a different order than that shown, or at the same time. Furthermore, some of these steps may be optional in some embodiments. Accordingly, the process 200 is merely exemplary of one process in accordance with illustrative embodiments. Those skilled in the art therefore can modify the process as appropriate.

The process 200 begins at process marker A to proceed to step 202, which determines time information. For example, the IPG 12 or external computer 210 may have a clock configured to determine the time in the patient's time zone. The time information may be updated regularly (e.g., in real-time, or in time intervals, such as in 1-minute, or 5-minute increments).

At step 204, the process asks whether the time determined in step 202 is within a pre-determined sleep time period. In other words, the process checks the clock to determine if the time of day corresponds to a pre-determined sleeping time (i.e., a time during which the patient is expected to be sleeping). The time during which the patient is expected to be sleeping may be a time during the night, or may be a time programmed into the IPG 12 that is tailored to the patient. For example, if the patient regularly sleeps during the day, the pre-determined sleeping time may include daylight hours or if the patient regularly sleeps during the night, the pre-determined sleeping time may include evening and nighttime hours. The pre-determined sleeping time may be a set of day and time range indications. Each indication may be of a format such as “Monday 9 PM to Tuesday 8 AM,” or “Sunday 10 AM to Sunday 6 PM,” or “Friday 12 AM to Friday 9 AM,” to name a few illustrative examples not limiting of the disclosure.

Alternatively, at step 204 the process may ask whether the time determined is in a pre-determined non-sleep time period and proceed to the step 206 or circle back to step 202 based on a negative determination, or proceed to step 208a based on a positive determination.

If the time is within a pre-determined sleep time period (or outside of a pre-determined non-sleep time period), then the process optionally proceeds to step 206, which enters a sleep apnea therapy mode. In the sleep apnea therapy mode, the IPG 12 is programmed to initiate disordered breathing therapy (e.g., therapy for CSA, OSA, combination apneas, or other disordered breathing) with a delivery of stimulation energy to one or more target nerves during sleep time periods. Alternatively, in some embodiments, the IPG 12 may not include a programmed sleep apnea therapy time period or sleep-apnea-therapy mode or may have a firmware or hardware setting that disable, temporarily or permanently, the sleep apnea therapy mode. In such a case, the IPG 12 may be a dedicated daytime, or awake time, therapy mode device and may circle back to the step 202 in lieu of entering a sleep-apnea therapy mode.

Within the sleep-apnea therapy mode, the process proceeds to step 212 to evaluate non-time based sleep indicators. The process may initiate neurostimulation based on these criteria in addition to the time evaluation.

For example, at the stage 212, they process may determine whether motion and/or position data indicate that the patient is asleep during the pre-determined sleep time period. The process may include an evaluation of patient activity and/or patient position criteria (e.g., as measured by an accelerometer such as the accelerometer 36 shown in FIG. 11A). The activity evaluation may be based on patient motion and the position evaluation may be based on patient pitch and/or body position (e.g., supine, prone, etc.). For example, if the motion and position data indicate one or more of motion below a pre-determined threshold (e.g., inactivity of the patient), a pitch below a pre-determined threshold, or a body position corresponding to a know sleeping configuration for the patient, then process determines that the motion and position data are consistent with a sleeping patient. Additionally or alternatively, the evaluation that sleep conditions are satisfied may include, among other things, monitoring physiological signals such as respiratory patterns (which become slower and more regular during sleep), and/or heart rate variability.

If sleep conditions are not satisfied, the process returns to step 204 and is repeated. If sleep conditions are satisfied, the process continues to step 214a, which asks whether to begin asynchronous stimulation in the sleep mode.

In some embodiments, the device determines whether to operate in synchronous or asynchronous stimulation mode (referred to as a sub-mode because synchronous or asynchronous stimulation can occur in either of the operational modes) based on a programmed setting for the IPG 12. For example, a stimulation profile for sleep disordered breathing therapy provided to the IPG 12 as firmware may include a sub-mode setting. In synchronous sub-mode, the device detects the onset of inspiration and delivers stimulation synchronously with and triggered by this detection. Asynchronous sub-mode, on the other hand, provides stimulation at preset intervals and without a trigger from the patient's respiratory signals occurring in real-time during therapy. In the synchronous sub-mode, the device may provide an asynchronous back-up mode in which the device provides stimulation at preset intervals in the absence of any detected respiration for a particular time period. For example, the particular time period may be 3-10 seconds where a lack of respiration for this time period indicates an apnea. Thus this asynchronous back-up mode may be referred to as a back-up apnea triggered asynchronous sub-mode.

Thus, depending on the situation, the process proceeds either to step 218a and provides synchronous stimulation, or proceeds to step 216a and provides asynchronous stimulation. The process repeats until it is determined at step 204 that the patient is not in a pre-determined sleep time period. At step 204, it may be determined that the present time is not within the pre-determined sleep time period (e.g., when sleep apnea therapy may optionally take place). At this point, the process proceeds to step 208a, which enters a non-sleep time conditioning mode.

In an embodiment, the system 5 may enter the sleep apnea therapy mode of the non-sleep time conditioning mode based on a user input to the patient-specific computing device 215. In response to this selection, the process may initiate neurostimulation based on patient activity and/or motion criteria (e.g., as measured by an accelerometer).

It should be noted that regardless of whether the process enters 206 the sleep-apnea therapy mode at the step 206 based on an automatic assessment of time or a patient selection at a user interface, the initiation of stimulation would need to be automatic based on either a pre-programmed time and/or a sensor signal such as an accelerometer signal. This is because the neurostimulation energy provided for a sleeping patient would likely cause discomfort in an awake patient. Therefore, the patient cannot both be asleep and capable of initiating the neurostimulation. In other words, the neurostimulation initiation for sleep time neurostimulation would not be under conscious control of the patient. In contrast, and as discussed in further detail with regard to the non-sleep time conditioning mode, in this latter mode, the system 5 can provide for initiation of neurostimulation in response to a patient input to a user interface because this mode is designed specifically for the awake patient providing conscious control of neurostimulation.

In various implementations, the IPG 12 may be implanted in a patient diagnosed with sleep apnea and/or a patient that may benefit from daytime (non-sleep time) respiratory conditioning and/or a patient diagnosed with atrial fibrillation (AF). The patient with sleep apnea may or may not present with a co-existing condition such as AF. Similarly, the patient with AF may or may not present with a co-existing condition such as sleep apnea.

As known to one of skill in the art, regulation of the respiratory cycle, during treatment of sleep apnea and/or during daytime conditioning, has the potential to improve the sympathetic and/or parasympathetic balance by decreasing the respiratory rate of the patient and creating longer, deeper breaths that in turn increase vagal tone. This improvement in vagal tone may decrease a patient's heart's susceptibility to AF. Thus, sleep apnea therapy and/or daytime conditioning, as described herein, may produce additional improvements in health with regard to AF beyond the improvements specific to sleep apnea without an adjustment of the performance of the system 5 specifically intended for or targeted to AF. Thus the process 200 as shown in FIG. 2A-1 may be implemented as a system and/or method for treating a sleep apnea and/or atrial fibrillation without any selection within the system 5 of a patient presenting with atrial fibrillation. In other words, the sleep apnea patient that also presents with atrial fibrillation may see incidental improvement in heart health.

In some examples, and as discussed with regard to FIG. 2A-2, the system 5 may include a capability to select various modes of operation based on a patient diagnosis of sleep apnea and/or AF For example, the system 5 may store a patient therapy mode indicator 62 (as shown, for example, in FIG. 11B) based on the patient diagnosis. The method 275 shown in FIG. 2A-2 may include additional other steps and some of the steps may be performed in a different order than that shown, or at the same time. Furthermore, some of these steps may be optional in some embodiments. Accordingly, the process 275 is merely exemplary of one process in accordance with illustrative embodiments. Those skilled in the art therefore can modify the process as appropriate.

The process 275 begins at step 240 which determines a patient therapy mode. For example, a processor of the IPG 12 (e.g., the processor 50) and/or external computer 210 may access a stored indication of a treatment mode of sleep apnea 242, AF 244, or AF+Sleep Apnea 246. In this way, the IPG 12 may be pre-programmed (e.g., configured in software and/or firmware) to provide stimulation to the phrenic nerve according to various stimulation profiles for sleep apnea and/or atrial fibrillation. In an example, the patient therapy mode controller 199, described in regard to FIG. 11B, may implement the step 240. Thus the system 5 may include a programmable setting (e.g., the patient therapy mode indicator 62 shown in FIG. 11B) to indicate a selected patient treatment mode of sleep apnea, AF, and/or AF+sleep apnea.

If the processor determines at the step 240 that the treatment mode is sleep apnea 242, then the system 50 may proceed to process marker A for the method 200 shown in FIG. 2A-1. As discussed above, the method 200 provides sleep apnea therapy and/or daytime conditioning. Further, implementation of the method 200 may incidentally benefit AF without specifically targeting AF based on a programmed setting.

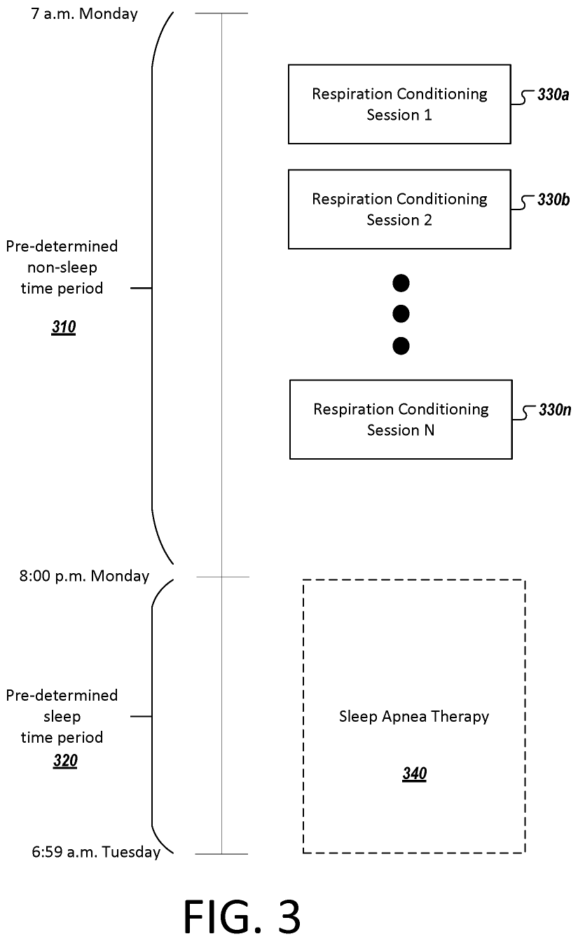

If the processor determines at the step 240 that the treatment mode is AF 244, then the system 50 may proceed to process marker B for the method 280 shown in FIG. 2A-3. As discussed below, the method 280 provides AF targeted therapy and/or daytime conditioning. If the processor determines at the step 240 that the treatment mode is AF+sleep apnea 246, then the system 50 may proceed to process marker C for the method 285 shown in FIG. 2A-4. As discussed below, the method 285 provides AF plus sleep apnea therapy and/or daytime conditioning.

FIG. 2A-3 shows a process 280 of determining an atrial fibrillation operational mode for sleep time and non-sleep patient control operational modes of the system of FIG. 1 in accordance with illustrative embodiments. For example, the processor 50 may perform steps of the process 280. The operational mode may be one of an AF therapy mode or a patient control mode for respiratory conditioning. The patient control mode for respiratory conditioning may provide therapeutic benefits for AF. The method shown in FIG. 2A-3 may include additional other steps and some of the steps may be performed in a different order than that shown, or at the same time. Furthermore, some of these steps may be optional in some embodiments. Accordingly, the process 280 is merely exemplary of one process in accordance with illustrative embodiments. Those skilled in the art therefore can modify the process as appropriate.

The method 280 is substantially as described herein for the method 200 for the steps 202 and 212 The steps 210a, 208b, 214b, 216b, and 218b of the method 280 may replace, respectively, the steps 206, 208a, 214a, 216a, and 218a of the method 200. The step 208b is described below with regard to FIG. 2B.

At the step 209, the method 280 may proceed as described in step 204 of the process 200 and determine whether the time is within a pre-determined sleep time period and then divert to the step 210a or the step 208b based on this determination. Alternatively, where the IPG 12 runs according to the process 280 in the case of an AF therapy mode determination at the step 240 of the process 275, at the step 209, the method 280 may pause stimulation therapy during the pre-determined sleep time period and only deliver stimulation therapy during the non-sleep time. As a further alternative, where the IPG 12 runs according to the process 280 in the case of an AF therapy mode determination at the step 240 of the process 275, at the step 209, the method 280 may pause stimulation therapy during the pre-determined non-sleep time period and only deliver stimulation therapy during the sleep time. The decision to deliver sleep time therapy without non-sleep time therapy (i.e., bypass non-sleep time therapy) or to deliver non-sleep time therapy without sleep time therapy (i.e., bypass sleep time therapy) may be based on the atrial fibrillation operation mode determination at the step 240 and/or may be based on an evaluation of AF metrics. In an example, the IPG 12 may access the cardiac status indicator 63 at the stage 209, and then bypass the sleep operational mode or the non-sleep time conditioning mode based on the cardiac status indicator 63. If the cardiac status indicator 63 indicates an improvement in AF metrics and/or a therapy bypass instruction, then the IPG 12 may bypass the sleep operational mode or the non-sleep time conditioning mode. Conversely, if the cardiac status indicator 63 indicates a deterioration in AF metrics and/or an instruction to enter sleep time therapy or non-sleep time conditioning mode, then the IPG 12 may enter the appropriate mode according to the time information. In the case of the bypass of either the non-sleep time conditioning mode or the sleep time AF therapy mode, the IPG 12 will pause delivery of any stimulation to the phrenic nerve for the duration of the pre-programmed sleep time or non-sleep time according to the time information determined at the step 202 in order to achieve the bypass.

For instance, the IPG 12 may access a stored cardiac status indicator 63 providing the evaluation of the AF metrics and/or an instruction to enter or bypass the sleep operational mode. In an example, the IPG 12 may bypass the sleep operational mode and provide only non-sleep time therapy. The IPG 12 may enter the sleep operational mode based on the cardiac status indicator 63 indicating an deterioration in AF status and/or an instruction to resume sleep time therapy. In an example, the IPG 12 may monitor signals from one or more cardiac sensors. If the signals from the one or more cardiac sensors indicate an improvement in AF or other cardiac indicators relevant to or correlated with AF, over some pre-determined monitoring time period, then the IPG 12 may continue to run only non-sleep time stimulation therapy. However, if the if the signals from the one or more cardiac sensors indicate no improvement or a deterioration in AF or other cardiac indicators relevant or correlated with AF, over that pre-determined monitoring time period, then the IPG 12 may initiate sleep time therapy. The IPG 12 may continue the sleep time therapy until the signals indicate an improvement in AF or other cardiac indicators relevant to or correlated with AF or may continue for a pre-determined period of time and then re-evaluate the cardiac signals. Such a controlled provision of sleep time therapy may preserve battery life and may avoid difficulties with patient adaption to sleep time therapy. In an example, a caregiver may monitor signals from one or more cardiac sensors, included with the system 5 and/or external to the system 5, and enter the cardiac status indicator via the external computing system 210. In an example, the patient therapy mode controller 199 may receive and monitor one or more sensor signals and determine the cardiac status indicator and/or the therapy mode at the step 240 based on the sensor signals. Additionally or alternatively, the patient therapy mode controller 199 may monitor the cardiac status indicator and/or the patient therapy mode indicator, as provided by a caregiver, and determine the therapy mode at the step 240 based on these indicators. One or more of the controllers 152 and 154 may select the sleep stimulation profile or the conditioning stimulation profile based at least in part on the cardiac status indicator and/or the patient therapy mode indicator through direct access to the indicators 62 and/or 63 and/or based on instructions from the patient therapy mode controller 199 based on access to the indicators 62 and/or 63.

At the step 210a, the system 5 may enter an AF therapy mode for sleep time stimulation therapy from the IPG 12. The AF therapy mode for sleep time stimulation therapy may utilize stimulation parameters at the steps 214b, 216b, and 218b that are the same or different from the stimulation parameters utilized at the steps 214a, 216a, and 218a of the method 200. In some patients, sleep apnea therapy, as described herein, may produce additional improvements in health with regard to AF beyond the improvements specific to sleep apnea without an adjustment of the performance of the system 5 specifically intended for or targeted to AF. Therefore, even if a patient does not present with sleep apnea, a therapy regimen designed for sleep apnea treatment may still provide therapeutic benefits for AF.

As examples, a sleep time therapy targeted to AF, rather than sleep apnea, may differ from a sleep time therapy targeted to sleep apnea, albeit with potential incidental benefits for AF. Table 1 shows examples of parameters that may differ between a sleep apnea targeted sleep time therapy and an AF targeted sleep time therapy. Note that the stimulation rate refers to a rate at which the IPG 12 stimulates the phrenic nerve to cause a diaphragmatic contraction thus inducing a breath. Therefore, the stimulation rate is the stimulated breaths per minute (bpm). This may differ from an intrinsic, unstimulated breathing rate of the patient. For example, the stimulation parameters as stored in the sleep stimulation profile library 57B, as discussed in regard to FIG. 11B, may correspond to sleep or non-sleep parameters based on the examples in Table 1.

TABLE 1

| | Sleep Apnea targeted | |
|---|---|---|
| Parameter | therapy | AF targeted therapy |
| Stimulation rate (breaths | Higher stimulation rate | Slower stimulation rate |
| per minute) | than for AF targeted | than for sleep apnea |
| | therapy | targeted therapy |
| | e.g., 16-24 bpm | e.g., 10-18 bpm |
| | e.g., programmed based on | e.g., programmed to slow |
| | the intrinsic breathing rate | breathing to a target rate |
| | | beneficial for vagal tone |
| | | which may or may not be |
| | | based on the intrinsic |
| | | breathing rate (e.g., which |
| | | may be independent of a |
| | | patient's intrinsic breathing |
| | | rate) |
| Stimulation amplitude | Lower than for AF targeted | Higher than for sleep apnea |
| | therapy resulting in a lower | targeted therapy resulting |
| | tidal volume relative to AF | in a higher tidal volume |
| | targeted therapy; | relative to sleep apnea |
| | programmed to target | targeted therapy; |
| | normalizing breathing and | programmed to target |
| | oxygen levels throughout | creating a slow and deep |
| | sleep while maintaining | breath while maintaining |
| | patient comfort | patient comfort |
| | e.g., 3-7 mA | e.g., 4-8 mA |
| Sleep time stimulation | Entire sleep time as | Entire sleep time as |
| duration | determined at steps 204 | determined at steps 204 |
| | and 212 (e.g., a duty cycle | and 212 OR intermittently |
| | of 28-42% to provide | during the sleep time (e.g., |
| | therapy for 7-10 hours out | 5, 10, 15, 20, 25, or 30 |
| | of every 24 hours) | minute duration intervals) |
| | | (e.g., a duty cycle of |
| | | approximately 5-10% or 5- |
| | | 15% to provide therapy for |
| | | 1-3 hours out of every 24 |
| | | hours or 5-20% to provide |
| | | therapy for 1-5 hours out of |
| | | every 24 hours) |

The sleep time stimulation duration provided for an entire sleep time includes inhalation and exhalation periods where stimulation is applied to the phrenic nerve to induce inhalation and then removed to allow exhalation with the inhalation and exhalation cycle continuing for substantially the entire sleep time of the patient. In contrast, stimulations applied intermittently refers to groups of inhalation and exhalation cycles as induced by stimulation session 5-60 minutes in duration with pauses between the groups that are substantially longer than a breath cycle, for example, pauses that are 5-30 minutes, 30-60 minutes, or 5-60 minutes in duration. Thus, where the duty cycle of the IPG 12 for a sleep apnea therapy may be about 30% (i.e., running 8 hours out of every 24 hours), the duty cycle of the IPG 12 for AF therapy may be on the order of 5%-10% (i.e., running 1-2 hours out of every 24 hours or 5%-15% (i.e., running 1-3 hours out of every 24 hours). Factors guiding a stimulation profile vary between day and night therapy and based on the purpose of the therapy. For example, higher amplitudes within some of the exemplary ranges may produce deeper breaths but may also wake a sleeping patient. As another consideration, discomfort thresholds may be lower in an awake patient than a sleeping patient. Thus stimulation parameters may be tailored specifically to the patient sleeping state combined with the targeted physiological issue.

FIG. 2A-4 shows a process 285 of determining a combination of atrial fibrillation and sleep apnea operational modes for sleep time or non-sleep patient control operational mode of the system of FIG. 1 in accordance with illustrative embodiments. For example, the processor 50 may perform steps of the process 285. The operational mode may be one of a combined sleep apnea and AF therapy mode or a patient control mode for respiratory conditioning. The patient control mode for respiratory conditioning may provide therapeutic benefits for AF and/or sleep apnea. The method shown in FIG. 2A-4 may include additional other steps and some of the steps may be performed in a different order than that shown, or at the same time. Furthermore, some of these steps may be optional in some embodiments. Accordingly, the process 285 is merely exemplary of one process in accordance with illustrative embodiments. Those skilled in the art therefore can modify the process as appropriate.

The method 285 is substantially as described herein for the method 200 for the steps 202, 204, 212, 214a, 218a, and 216a. The step 208c described below with regard to FIG. 2B may replace the step 208a of the method 200. At the step 210b, the system 5 may enter an AF+sleep apnea therapy mode for sleep time stimulation therapy from the IPG 12. The AF+sleep apnea therapy mode for sleep time stimulation therapy may utilize stimulation parameters at the steps 214b, 216b, and 218b that are the same as the stimulation parameters utilized at the steps 214a, 216a, and 218a of the method 200. FIG. 2B shows a detailed process 250 of the non-sleep time conditioning mode 208a, 208b, or 208c as discussed in regard to FIGS. 2A-1, 2A-3, and 2A-4 for the system of FIG. 1 in accordance with illustrative embodiments described herein. For example, the processor 50 may perform steps of the process 250. The method shown in FIG. 2B may include additional steps and some of the steps may be performed in a different order than that shown, or at the same time. For example, steps 252 and 254 may occur simultaneously. Additionally, as another example, step 262 may lead directly to step 258 instead of step 256.

Furthermore, some of these steps may be optional in some embodiments. For example, steps 272-274 may be optional, and some embodiments may not include a sleep stimulation mode. Accordingly, the process 250 is merely exemplary of one process in accordance with illustrative embodiments. Those skilled in the art therefore can modify the process as appropriate.

The process begins at step 252, which determines that the patient is in a non-sleep time period. FIG. 3 schematically shows details of determining whether the patient is in a pre-determined sleep time period or in a pre-determined non-sleep time period. Accordingly, the patient and/or medical practitioner may input predetermined awake time period(s) and/or predetermined sleep time period(s).

For example, the system may be programmed, as an example, that 7 AM to 8 PM is a pre-determined non-sleep time period 310 and/or that 8 PM to 7 AM is a pre-determined sleep time period 320. Each of the periods 310 and 320 corresponds to a respective pre-programmed start time-of-day and a pre-programmed stop time-of-day. Thus, upon determination that the time has reached 7 AM on Monday, the process proceeds to step 254, which enters non-sleep time conditioning mode. The non-sleep time conditioning mode may also be referred to as a patient control operational mode. The patient control operational mode includes, or provides for one or more patient selectable respiration conditioning sessions 330a, 330b, . . . , 330n (e.g., session 1, session 2, . . . , session N as shown in FIG. 3). The patient may initiate the neurostimulation for each session. Once initiated, the patient may interact with the system 5 during the session and/or may terminate the neurostimulation for the session. In contrast, if the system determines that it is after 8 PM on Monday and before 7 AM Tuesday, the system may enter sleep operational mode. In sleep operational mode, the system may be inactive, or may provide sleep apnea therapy 340.

At step 256, the process receives a session activation signal to begin neurostimulation for a respiration conditioning session. The neurostimulation may commence in direct response to a user initiated session activation signal without a determination of any patient state or physiological status. In various embodiments, the session activation signal is received from the patient-specific computing device 215. To that end, a user interface 220 may be provided to the patient.

FIG. 4 schematically shows a user interface 220 of the patient control mode in accordance with illustrative embodiments described herein. The patient-specific computing device 215 may provide the user interface 220. The user interface 220 may include an activation button 222 that is selectable by patient input. Additionally, the user interface may include a delay button 224 selectable by patient input. When the patient is ready to begin a respiratory conditioning session, they may select the activation button 222 to provide the activation signal to the system 10. At this point in the process, as an example, respiratory conditioning session #1 begins.

Unlike passive stimulation during sleep, awake sessions rely on the patient's conscious involvement to synchronize their natural breathing efforts with the stimulator's electrical impulses. Further these conscious sessions train the patient not to fight or resist the stimulation and help to reduce psychological and/or physical discomfort associated with neurostimulation, particularly with unilateral stimulation where the patient is able to resist stimulation. Further a patient using the conditioning regime has the necessary muscular control to breathe without stimulation thus further enabling the effectiveness of unilateral stimulation. In contrast, a patient requiring a ventilator or suffering from a loss of muscular control due to a condition like paralysis or amyotrophic lateral sclerosis (ALS) may require bilateral stimulation and may lack the ability to override or resist the stimulation as they may be unable to breathe in the absence of stimulation. Commencing stimulation upon receipt of the activation signal from the patient is possible in the awake mode because the system 5 provides neurostimulation compatible with an awake patient without creating discomfort. Additionally, the neurostimulation in this mode may be below a threshold to contract the diaphragm and may merely serve as a prompt to the patient to provide the physiological benefits of slower and deeper breaths and/or to acclimate the patient to the sensation of neurostimulation and to the experience of breathing in rhythm with neurostimulation. Additionally, the signal confirms that the patient is prepared and in a suitable state to proceed, minimizing discomfort and ensuring safety. This patient-led initiation also fosters engagement and compliance, key factors in achieving long-term respiratory improvement.

Returning to FIG. 2C, after receiving the session activation signal, the process continues to step 257, which selects a conditioning stimulation profile (e.g., as discussed further herein, for example, at least in regard to FIG. 11B). The processor may select a conditioning stimulation profile having a synchronous sub-mode or an asynchronous sub-mode. Preferably, an asleep time synchronous sub-mode includes the back-up apnea triggered asynchronous sub-mode. In contrast, the awake time synchronous sub-mode does not include the backup mode. Additionally, in an implementation the processor may select a conditioning profile, with either a synchronous or asynchronous delivery mode and depending on the patient therapy mode indicator 62 as determined at the step 240. Thus, each of 208a, 208b, and 208c may correspond to the same process 250 with the exception of a selection of the particular conditioning stimulation profile. For example, the mode 208a may include selection of a conditioning stimulation profile without reference to a patient therapy mode indicator 62 (e.g., in the absence of the entry of such an indicator) or may include selection of a conditioning stimulation profile targeted for sleep apnea therapy. The mode 208b may include selection of a conditioning stimulation profile without reference to a patient therapy mode indicator 62 (e.g., in the absence of the entry of such an indicator) or may include selection of a conditioning stimulation profile targeted for AF therapy. The mode 208c may include selection of a conditioning stimulation profile without reference to a patient therapy mode indicator 62 (e.g., in the absence of the entry of such an indicator) or may include selection of a conditioning stimulation profile targeted for sleep apnea and AF therapy. The process then continues which provides stimulation for the respiration conditioning session 259 in accordance with the selected mode and stimulation profile.

Table 2, below, shows examples of different sub-mode combinations for awake time respiratory conditioning with asleep time sleep-apnea treatment on any given day of treatment. The various available sleep-apnea treatment stimulation profiles and conditioning stimulation profiles may cause the IPG to provide stimulation according to different sub-modes (e.g., synchronous or asynchronous). Each of the asynchronous sub-mode and the synchronous sub-mode may be used for awake stimulation or for sleeping stimulation, such as a sleep disordered breathing stimulation mode. However, while the sub-mode may be the same for an awake period as for a sleep period, stimulation parameters (examples of which are provided below in Table 3) may not be the same for an awake period and a sleep period. The system 10 may determine whether to enter an asynchronous stimulation or synchronous stimulation sub-mode based on programming and/or other settings and/or may be based on instructions from the external computing device 210 and/or the patient-specific external computing device 215. For a given patient, the system 10 may switch between asynchronous and synchronous stimulation at various times. For example, the system 10 may provide asynchronous or synchronous stimulation while the patient is awake, and may provide asynchronous or synchronous stimulation while the patient is asleep, but the stimulation sub-mode provided while the patient is awake may or may not be the same as the stimulation sub-mode provided while the patient is asleep.

The sub-modes shown below are the available sub-modes for (a) the patient control operational mode, or non-sleep time conditioning mode, for the awake patient where stimulation is provided for respiratory conditioning and (b) the sleep operational mode for the sleeping patient where stimulation is provided to treat sleep disordered breathing.

TABLE 2

| | Patient | | |
|---|---|---|---|
| | Status | Sub-Mode Description | Start Condition |
| Example | Asleep | Asynchronous sub-mode | Motion below sleeping |
| 1 | | | threshold & Time |
| | Awake | Asynchronous sub-mode | Time & Patient activation |
| Example | Asleep | Asynchronous sub-mode | Motion below sleeping |
| 2 | | | threshold & Time |
| | Awake | Synchronous sub-mode | Breath detection & Time & |
| | | (excludes back-up apnea-triggered | Patient activation |
| | | asynchronous sub-mode) | |
| Example | Asleep | Synchronous sub-mode | Motion below sleeping |
| 3 | | (includes back-up apnea-triggered | threshold & Time & Breath |
| | | asynchronous sub-mode) | detection |
| | Awake | Synchronous sub-mode | Breath detection & Time & |
| | | (excludes back-up apnea-triggered | Patient activation |
| | | asynchronous sub-mode) | |
| Example | Asleep | Synchronous sub-mode | Motion below sleeping |
| 4 | | (includes back-up apnea-triggered | threshold & Time & Breath |
| | | asynchronous sub-mode) | detection |
| | Awake | Asynchronous sub-mode | Time & Patient activation |

In various examples, the asleep and awake stimulation (e.g., as controlled by the awake profiles 58 and asleep profiles 60 as shown in FIG. 11B) may differ in other ways than the sub-mode changes. For instance, the reduction in breathing rate when awake could be much lower than when asleep. Thus in a range of 8-30 bpm available from the IPG 12 based on stimulation rate, the awake stimulation profiles 58 may stay in an 8-14, 10-14, 8-18, or 10-18 bpm range whereas the asleep stimulation profiles 60 may stay closer to an intrinsic breathing rate in the 16-24 bpm range. As another example, the stimulation amplitudes may be higher for the asleep stimulation profiles 60 than for the awake stimulation profiles 58 because patients are better able to tolerate higher amplitudes when asleep. As a further example, the inspiration to expiration (I:E) ratio may vary along with amplitude to provide a deeper inspiration and longer expiration (e.g., I:E of 1:2 or 1:3) in an awake stimulation profile 58 than in the asleep stimulation profile 60 (e.g., I:E of 1:1).

Illustrative embodiments provide three primary sub-modes of IPG operation used in respiratory therapy: asynchronous, synchronous, and back-up asynchronous. In asynchronous sub-mode, stimulation is delivered at a fixed, pre-set rate based on the patient's intrinsic breathing pattern without stimulation. This mode does use detection of inspiration to trigger stimulation. In contrast, in the synchronous sub-mode, breath detection triggers stimulation. This mode is especially helpful during the day when the patient is awake and can respond to the breath pacing, it also supports breath depth acclimation by training the patient to tolerate breath holds and develop deeper, slower breathing.

The synchronous sub-mode of IPG operation may be a sleeping time period synchronous sub-mode or a non-sleeping time period synchronous sub-mode. The sleeping time period synchronous sub-mode includes a third sub-mode, back-up asynchronous which is a apnea triggered asynchronous mode. In the sleeping time period synchronous sub-mode, if the IPG does not detect a breath for a period of 3-10 seconds, it automatically switches to asynchronous delivery, ensuring that stimulation continues even in the absence of breathing effort. In the sleep time modes, back-up asynchronous may be applied if apnea is detected (i.e., no breath for 3-10 seconds) during synchronous sub-mode, because breath detection triggers stimulation in the synchronous sub-mode. Therefore, if no breath is detected, then stimulation starts at a pre-determined stimulation rate.

In contrast, the non-sleeping time period synchronous sub-mode excludes the apnea triggered asynchronous sub-mode. During the non-sleeping time period and during stimulation for respiratory conditions, patients are, or should be, conscious and capable of initiating a breath. Furthermore, during the non-sleeping time period, an awake and conscious patient may necessarily refrain from breathing for at least 3-10 seconds while talking, eating, drinking, etc.

As shown in Table 2, time is a start condition for neurostimulation in all sub-modes. As discussed at least in regard to FIGS. 2A-1, 2A-3, 2A-4, and 3, the processor 50 may be programmed with a pre-determined sleep time period 320 and a pre-determined non-sleep time period 310. Thus, the applied sub-mode needs to correspond to the designation of a particular time as a sleep time or a non-sleep time (an awake time).

As additionally shown in Table 2, and as discussed at least in regard to FIGS. 2A-1, 2A-3, 2A-4, and 3, for all sub-modes provided when the patient is asleep, a determination that motion is below a sleeping threshold is a start condition for neurostimulation. As further shown in Table 2, and as discussed at least in regard to FIG. 2B, patient activation, or the receipt by the processor 50 of a session activation signal, is a start condition for neurostimulation for all sub-modes provided when a patient is awake. Finally, as shown in Table 2, for synchronous sub-modes, provided either when the patient is asleep or when the patient is awake, the trigger for neurostimulation, and thus a necessary start condition, is detection of breath.

Respiratory conditioning may provide several benefits: rate acclimation, breath depth and rate training, sensation acclimation, sympathovagal balance, and/or improvement in AF metrics. Rate acclimation refers to training the body to accept a set breathing pace. The asynchronous sub-mode may be of particular benefit in this regard as the stimulation rate is pre-determined and not modulated in by detected breathing in the manner of the synchronous sub-mode. Breath depth and rate training refers to neurostimulation that trains the patient to take slower and deeper breaths. Such breathing is beneficial to overall health, particularly for patients presenting with central sleep apnea. The synchronous sub-mode may be of particular benefit in this regard as the stimulation may be triggered by inhalation and then may effect a hold between inhalation and exhalation thereby slowing down breathing. Further, the neurostimulation may effect various degrees of diaphragmatic response to effect deeper breaths at the slower rate. Sensation acclimation involves helping patients become comfortable with sensation of neurostimulation and of respiratory modulation. Some patients are apprehensive about neurostimulation at night and/or try to resist the breath hold sensation. Both the synchronous and asynchronous sub-modes may be of particular benefit in this regard. Thus, in addition to health benefits directly from the daytime respiratory conditioning, such conditioning may improve efficacy of sleep disordered breathing therapy. Faster acclimation to the sleep-disordered breathing therapy may enable patients to reach optimum stimulation energies and stimulation durations faster without discomfort or awakening. In turn, this may enable patients to more reduce or eliminate sleep apneas at night more efficiently and over a shorter time period. Further, as discussed herein, regulation of the respiratory cycle, during daytime conditioning, has the potential to improve the sympathetic and/or parasympathetic balance by decreasing the respiratory rate of the patient and creating longer, deeper breaths that in turn increase vagal tone. This improvement in vagal tone may decrease a patient's heart's susceptibility to AF.

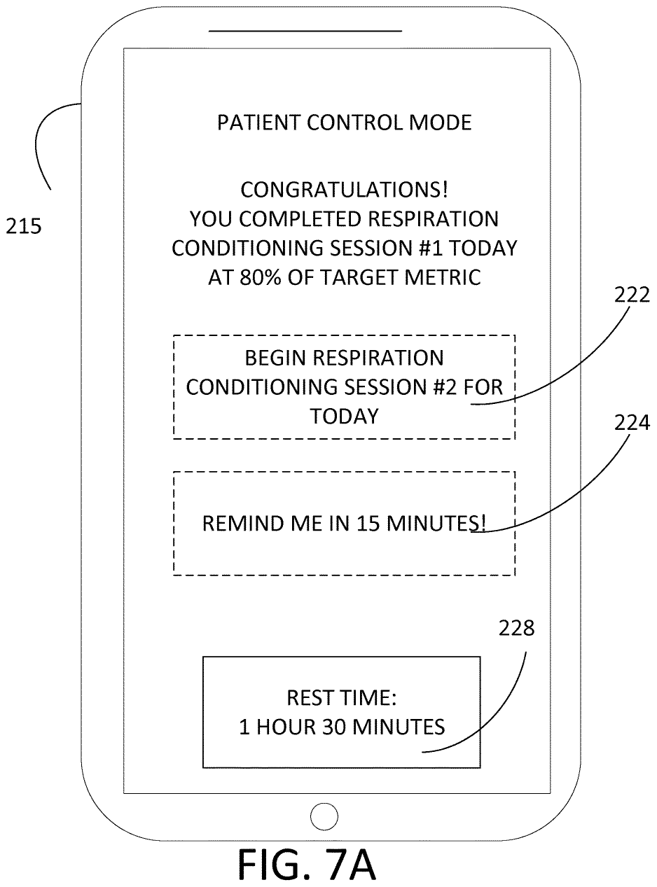

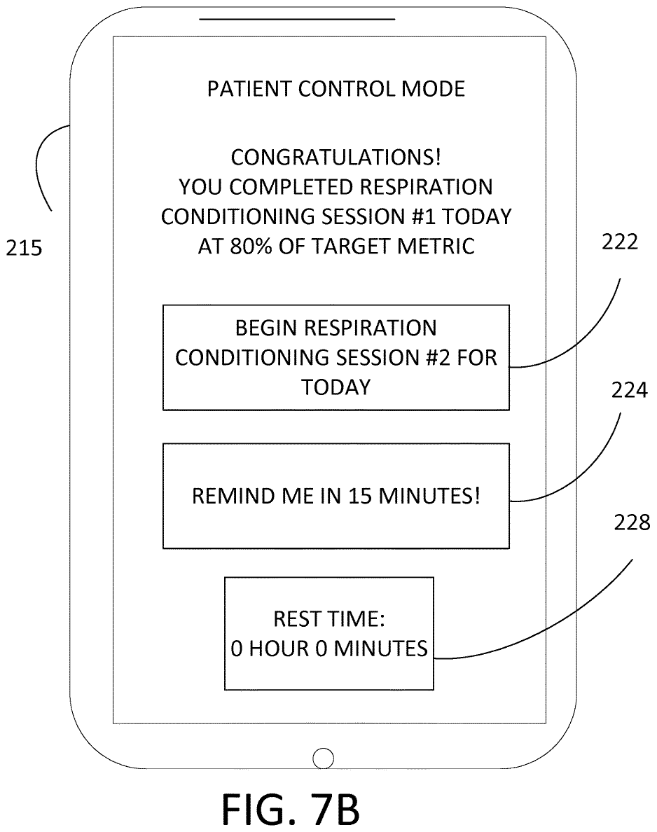

Returning to FIG. 2B with further reference to FIG. 7A, the process 250 continues to step 268 where the respiration conditioning session is completed. FIG. 7A schematically shows the user interface 220 of the patient control mode when the respiratory conditioning session is completed in accordance with illustrative embodiments described herein. The user interface may display a message indicating that the session was completed, and may include various performance parameters, including a patient compliance rate. The compliance rate may be tracked by the IPG processor and/or the mobile device of the patient (e.g., a mobile device having a sensor for measuring respiration). If there are additional respiratory conditioning sessions, the user interface may display the activation button 222. However, in some embodiments, the medical practitioner may require that the respiratory conditions be spaced out some minimum amount of time. Accordingly, there may be a refractory period, or a rest period, during which a subsequent respiration conditioning session may not be activated. In various embodiments, the rest period may be a function of a patient metric, such as patient compliance in the previous respiration conditioning session being above a threshold. If the patient performance is above the threshold, then a first pre-determined amount of time may pass before a subsequent respiration conditioning session activation button 222 becomes selectable. If the patient performance is below the threshold, then a second pre-determined amount of time (preferably longer than the first period of time) may pass before a subsequent respiration conditioning session activation button 222 becomes selectable. The user interface may display the rest period remaining 228.

The process then continues to step 270, which asks whether there are more respiration conditioning sessions for the current awake period. If yes, the process returns to step 256. Returning to the example of FIG. 3, the process would proceed to respiration conditioning session 2. The subsequent respiration conditioning session has an associated respiratory conditioning stimulation profile that may be pre-programmed by a medical practitioner based on patient clinical profile. For example, subsequent stimulation profiles may be configured to increase the intensity of the stimulation so as to acclimate the patient to the sensation of stimulation.

In some embodiments, subsequent stimulation profiles may be configured to increase the intensity of stimulation—such as by raising the amplitude or prolonging the stimulation duration—to gradually condition the patient's tolerance and respiratory coordination. Conversely, subsequent profiles may be made easier to complete by reducing the amplitude of stimulation, shortening the overall profile duration, decreasing the respiratory rate, or modifying the stimulation ramp characteristics to allow a gentler onset and offset. These adjustments can help patients who are struggling to meet compliance targets or who require more gradual acclimation. In some embodiments, the subsequent stimulation profile may begin immediately following the early termination of the first profile, while in others it may begin at its regularly scheduled time.

In some embodiments, a requisite rest period may need to pass before a subsequent respiration conditioning session may begin. FIG. 7B schematically shows the user interface 220 of FIG. 7A after the requisite rest period has expired. As shown, the buttons 222, 224 in the user interface may now be selectable.

In some embodiments, the process stops at step 271 prior to returning to step 256. Alternatively, the subsequent sessions may be pre-programmed and not changeable without the intervention of a medical practitioner. At step 271, subsequent respiration conditioning stimulation profiles are adjusted for the patient control operational mode as a function of feedback from the prior respiration conditioning sessions.

In various embodiments, feedback from the patient (e.g., the controls) or sensors obtained during a prior respiratory conditioning session (e.g., at steps 261-266) may be used to adjust subsequent respiratory conditioning sessions. For example, when the patients request lower stimulation or sensors detect signs of distress such as rapid breathing, increased heart rate, or irregular muscle activity, this data can be analyzed to identify thresholds where stimulation becomes uncomfortable or counterproductive. Using this information, future stimulation profiles can be modified to start at lower intensities, increase ramp-up durations, or fine-tune stimulation frequency, ensuring the regimen remains both effective and tolerable. Over time, as the patient adapts, the system can gradually increase the challenge to improve and/or expedite patient acclimation to therapy and/or enhance diaphragm strength and endurance without exceeding comfort limits.

As an example, Stimulation Profile 1 may use a stimulation amplitude of 2.5 mA, and Profile 2 may be originally configured for 3.0 mA. Based on subject patient feedback indicating that 2.5 mA was already perceived as strong, and objective feedback that Profile 1 was just barely completed, Profile 2 may be automatically or manually adjusted to a lower intensity of 2.75 mA to remain within the patient's tolerability range. Alternatively, if proceeding with the originally planned 3.0 mA amplitude, Profile 2 may be modified by shortening its duration—by 50% or even 75%—to reduce the overall stimulation burden while still introducing a higher-intensity challenge. These types of adaptive adjustments enable a personalized therapy progression that balances efficacy with patient comfort and compliance.

Similarly, objective feedback regarding the patient's ability to follow breathing instructions allows the system to refine these patterns for future sessions. If a patient struggles to achieve specific target rates or depths (also referred to as a respiratory goal), the system can adjust the targets to align with their current capabilities, ensuring better compliance and ease of execution. Additionally, objective biofeedback data helps the system set dynamic safety thresholds for stopping or adjusting stimulation in subsequent sessions, minimizing the risk of discomfort or distress. Progress tracking, such as trends in reduced distress markers or improved compliance rates, informs the gradual intensification of the regimen, helping the patient achieve measurable improvements in respiratory function. By continuously adapting to feedback, the system advantageously profiles safer, more comfortable, and more effective subsequent respiratory conditioning sessions.

If there are no more respiration conditioning sessions for the current awake period, the process continues to step 272, which determines that the patient is in a sleep time period. The system may enter the sleep operational mode during the patient's designated sleep hours. The system may monitor the patient's body position and activity level, ensuring activation occurs only when the patient is reclined and at rest. If the patient moves, sits up, or gets out of bed, the therapy pauses and may resume once the patient returns to a reclined, motionless state.

Upon activation, the device delivers electrical stimulation to the phrenic nerve in accordance with a sleep stimulation profile. This process helps maintain consistent breathing patterns throughout the night, reducing apnea episodes and improving sleep quality. Patients may initially be aware of the diaphragm's movement due to stimulation, but most adjust to this sensation within the first few months of therapy.

At step 274, the sleep stimulation profile may be adjusted as a function of feedback from the earlier respiration conditioning session. Respiratory conditioning sessions that take place while the patient is awake can greatly enhance the effectiveness of phrenic nerve stimulation for sleep apnea during sleep, and also may be used make adjustments to the sleep stimulation profile. These respiratory conditioning sessions may help improve and/or expedite patient acclimation to therapy, strengthen the diaphragm, improve its endurance, and/or refine neuromuscular coordination. Over time, a well-conditioned diaphragm responds more effectively to stimulation, enabling consistent and efficient contractions during sleep. Moreover, the awake sessions allow the patient to adapt to the sensation of electrical stimulation, reducing nighttime disruptions. Based on feedback from successful respiration conditioning sessions, the intensity or frequency of the sleep stimulation profile can be optimized, ensuring that the diaphragm is stimulated at a level that maximizes therapeutic benefit without causing discomfort. For example, sleep stimulation intensity might be slightly increased if the respiration conditioning sessions indicate improved and/or expedited patient acclimation to therapy and/or improved diaphragm strength, allowing for enhanced respiratory support during sleep. Unsuccessful respiratory conditioning sessions (e.g., stopped or reduced) may signal the need for adjustments to the sleep stimulation profile to address potential challenges. If awake sessions reveal difficulties, such as inadequate diaphragm engagement or discomfort from the stimulation, the intensity of the sleep mode stimulation may need to be reduced to ensure the patient's comfort and minimize disruptions to sleep. Alternatively, the profile might incorporate a gentler ramp-up or lower baseline intensity to allow the diaphragm to gradually acclimate to the stimulation. Additionally, if the awake sessions indicate poor synchronization with breathing patterns, the frequency or timing of the sleep mode stimulation could be adjusted to better align with the patient's natural respiratory cycle. These refinements, informed by awake session outcomes, help to mitigate potential negative impacts on the effectiveness of sleep apnea therapy.

Additionally, the user of the respiration conditioning stimulation may advantageously step up the timeline for beginning sleep stimulation. Generally, there is a waiting period after IPG 12 implantation of about 4 to 6 weeks before CSA therapy begins. Early implementation of awake mode respiration conditioning sessions may allow for earlier initiation of CSA therapy (e.g., 1 to 3 weeks). In part this is due to the strengthening of patient respiration, but also because of expedited adjustment to stimulation sensations.

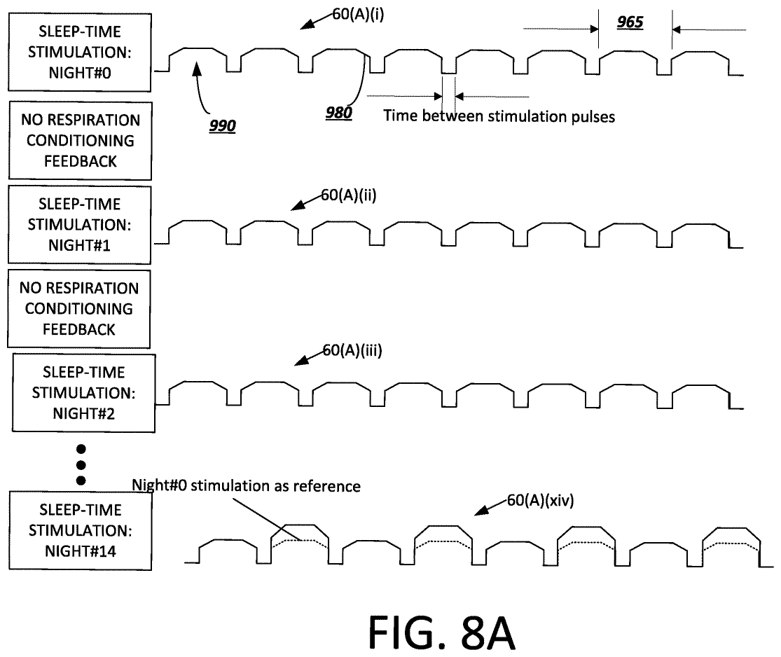

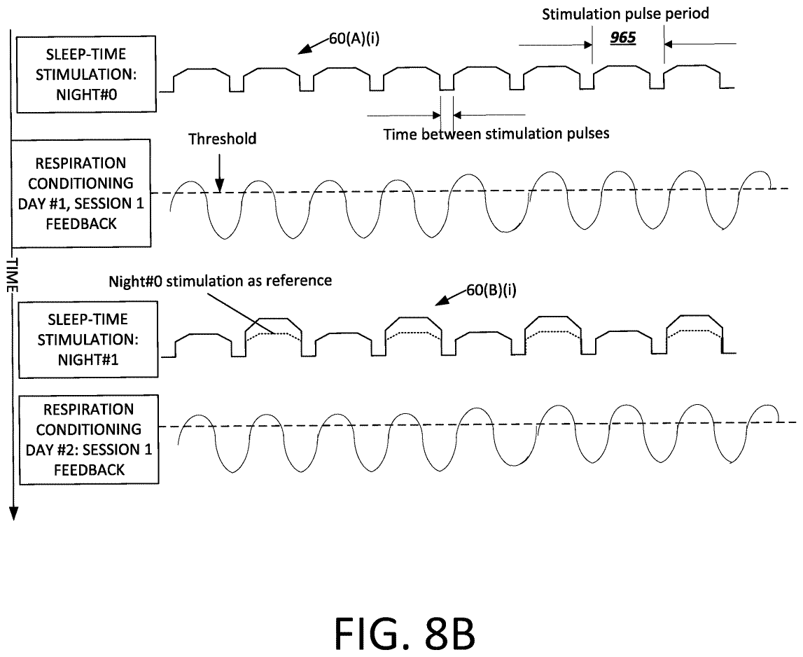

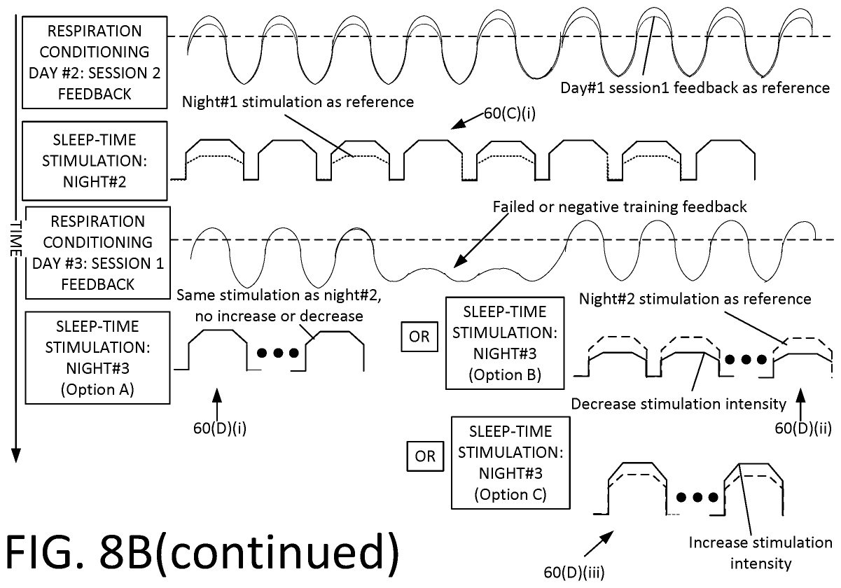

FIG. 8A schematically shows an example of sleep stimulation therapy without feedback from respiratory conditioning sessions. In this example, the medical practitioner decides that a patient should be stimulated using a given sleep stimulation profile 60(A) for 2 weeks (night #0-night #13). Thus, every night, in accordance with this plan, the patient may be stimulated according to the initial sleep stimulation schedule (e.g., 60(A)(i)-60(A)(xiii)). In this example, all of these stimulations remain the same until the 14th night, when stimulation 60(a)(xiv) is increased. This sequence of sleep stimulation profiles 60 takes place as assigned by a medical practitioner.

FIG. 8B schematically shows an example of sleep stimulation therapy using feedback from respiratory conditioning sessions to adjust sleep stimulation profiles in accordance with illustrative embodiments described herein. A first baseline sleep stimulation profile 60(A)(i) (night #0) may be provided to the patient as they sleep. The next morning during the patient control operational mode, the patient may have a first session of respiration conditioning (day #1, session #1) with a respiratory conditioning stimulation profile 58 (not shown). Feedback may be obtained from the session. In this example, the feedback indicates that the overall patient metric was above a threshold. Accordingly, the subsequent sleep-time stimulation (Night #1) may have an adjusted stimulation profile 60(B)(i). For the sake of discussion, the stimulation is shown as having increased alternating intervals, but it should be understood that this is merely for the sake of discussion. Those skilled in the art can determine a variety of ways of adjusting the stimulation profile 60 based on patient performance in the patient control mode.

During a subsequent respiration conditioning session (day #2, session #1) additional feedback may be obtained. Because of acceptable patient performance, the system 10 may provide a second respiration conditioning session on day 2 (day #2, session 2). Because both of the respiration conditioning sessions resulted in satisfactory patient performance (above the threshold) the sleep time stimulation 60(C)(i) on night #2 may be increased again.

The subsequent day, the patient may have another respiration conditioning session (day #3, session #1). The objective feedback from the session may show that patient performance was not adequate (e.g., below the threshold). Accordingly, the feedback may be said to be negative feedback. At this point, the subsequent sleep-time stimulation profile may be adjusted in a variety of ways. For example, in response to the negative feedback, the sleep stimulation profile may: remain substantially the same as the previous night stimulation profile parameters 60(D)(i) (Option A), decrease stimulation profile 60(D)(ii) parameters relative to the previous night (Option B), or increase stimulation profile 60(D)(iii) parameters relative to the previous night (Option C).

In various embodiments, the stimulation profile parameters may include stimulation intensity, pulse width, frequency, ramp-up duration, and ramp-down duration. These parameters may be adjusted in response to awake-session feedback, the system ensures that sleep therapy remains safe, effective, and aligned with the patient's evolving respiratory needs. This dynamic approach helps maximize therapeutic benefits while minimizing the risk of discomfort or poor adherence. For example, daytime respiratory conditioning may improve patient acclimation to sleep apnea therapy such that an average nighttime stimulation intensity may progress to a higher intensity over a shorter period of time (e.g., some period of days, weeks, or months) with daytime respiratory conditioning than without.

It should be understood that although the changes in the stimulation profile are shown as occurring in subsequent night time stimulation profiles, various embodiments may adjust more gradually. For example, the system may monitor performance over a longer period of time (as opposed to one particular respiratory conditioning session or the immediately prior respiratory conditioning session) and may change sleep stimulation profiles from week-to-week rather than night-to-night.

At step 276, the process adjust subsequent respiration conditioning stimulation profiles 58 for patient control operational mode as a function of feedback from the prior respiration conditioning session(s). This is similar to the process described at step 271, but does not necessarily occur during the same patient control mode session. However, it should be noted that the collected patient data may be provided to a medical practitioner who may make changes to subsequent respiration conditioning stimulation profiles. However, in some embodiments, the system may be programmed to make adjustments.

For example, if feedback indicates that the previous stimulation profile(s) caused discomfort or muscle fatigue, the intensity can be reduced in future sessions. Conversely, if the diaphragm responded well and the patient tolerated the intensity, the intensity can be gradually increased to enhance the conditioning effect. Similarly, if the timing or frequency of the pulses was not well-aligned with the patient's natural breathing patterns, adjustments can be made to synchronize the stimulation better with their respiratory cycle, improving diaphragm engagement.

As another example, biofeedback such as elevated heart rate or skin conductance during prior sessions might suggest that the ramp-up period for stimulation needs to be gentler, allowing the patient to acclimate more comfortably. If the patient reports difficulty achieving target breathing patterns, such as maintaining the instructed rate or depth, future sessions can simplify or adjust these instructions to align more closely with their current respiratory capabilities. For instance, the system may reduce the frequency of stimulation bursts or increase the rest intervals between pulses to facilitate easier compliance.

As yet another example, data from sensors measuring diaphragm engagement, such as electromyography (EMG), may indicate suboptimal muscle activation, parameters like pulse width or amplitude may be increased to enhance the muscle response. Conversely, if overstimulation leads to irregular muscle contractions or discomfort, these parameters can be reduced. Safety thresholds for automatic stimulation cessation can also be adjusted based on feedback, ensuring that the system stops stimulation earlier if similar signs of distress occur. By dynamically adjusting parameters such as intensity, frequency, pulse width, and ramp-up time in response to specific feedback, the therapy becomes personalized for the patient.

Thus, it should be understood that the process may be configured to adjust one or more stimulation parameters over successive respiration conditioning sessions. Adjustments may be based on feedback and parameters over a first group of one or more respiration conditioning sessions

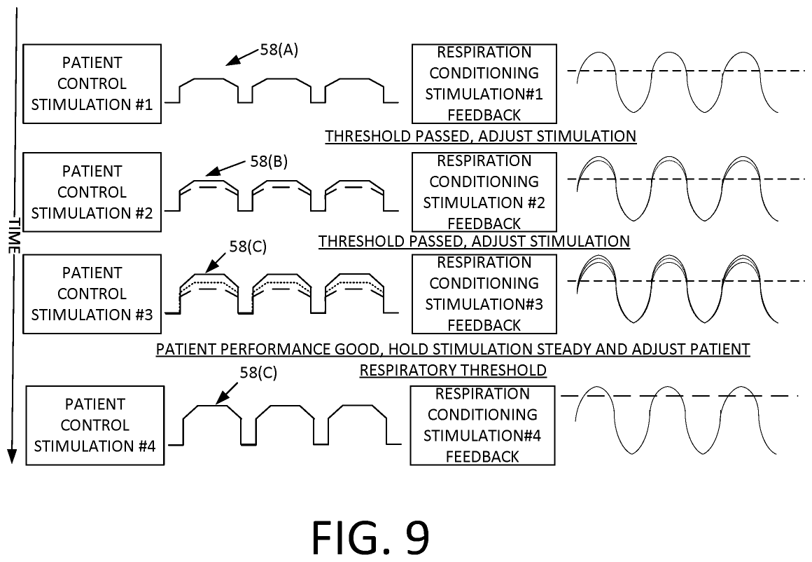

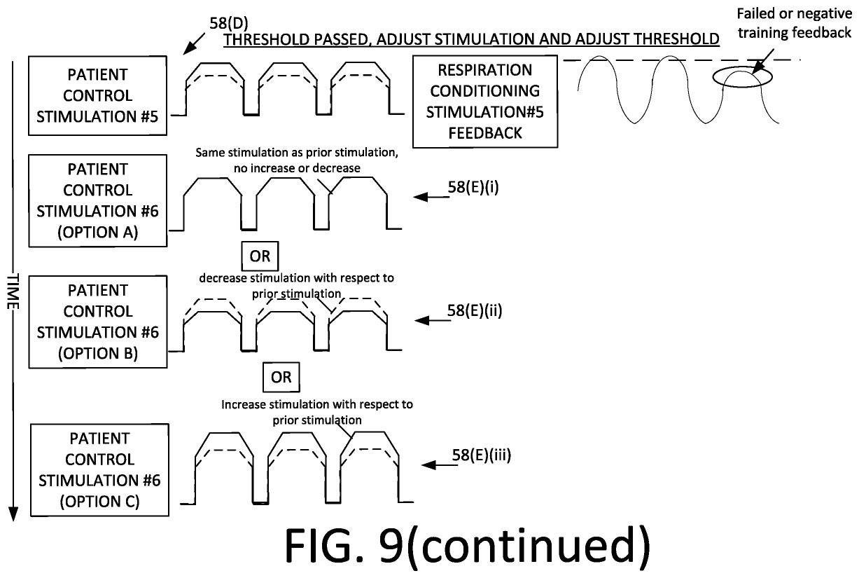

FIG. 9 schematically shows an example of feedback from one or more respiratory conditioning sessions being used to adjust stimulation profiles for one or more subsequent respiratory conditioning sessions in accordance with illustrative embodiments described herein. It should be understood that the general principles described here apply to adjustments made in both step 271 and step 276.

FIG. 9 begins by showing a baseline patient control stimulation profile 58(a), and a corresponding respiration conditioning feedback obtained from the patient while performing the first respiratory conditioning session. As shown, in this example, the respiration conditioning #1 feedback was successful (i.e., above threshold). The patient control stimulation #2 parameters may be slightly adjusted (e.g., increased as compared to prior profile shown in dashed lines). The new stimulation profile 58(b) is shown. Again, the corresponding patient feedback is successful (prior feedback shown in dashed lines). Accordingly, the stimulation profile may again be adjusted to third stimulation profile 58(c) (e.g., increased as compared to prior profile shown in dashed lines). Assuming patient feedback again is improved, the system 10 may adjust patient respiratory thresholds (e.g., represented by the increased threshold line). Thus, the stimulation profile 58(c) may remain the same at the respiratory conditioning session #4. Continued acceptable patient performance may result in continuously adjusted patient stimulation parameters (e.g., increased). In various embodiments, the system may require receiving approval from the patient's physician before increasing or adjusting the patient stimulation. The physician may receive updates from the patient's mobile device.

When the patient performance is not acceptable (e.g., represented by the failed or negative training feedback from the fifth patient control stimulation), the system 10 may make a number of adjustments. For example, the subsequent stimulation profile may, remain substantially the same as the previous respiration conditioning stimulation profile parameters (Option A, stimulation profile 58(E)(i)), decrease stimulation profile parameters relative to the previous respiration conditioning session (Option B, stimulation profile 58(E)(ii)), or increase stimulation profile parameters relative to the previous respiration conditioning session (Option C, stimulation profile 58(E)(iii)).

For the sake of discussion, the figure illustrates a single instance of failed or negative training feedback. It should be noted that a “failed” patient performance is not determined by a single breath. Instead, patient feedback is typically assessed based on an average or trend over time. In this context, there generally isn't truly a “failure” in training; rather, it is an indication that the training is not effectively producing a meaningful change in the patient's overall respiratory patterns, particularly during sleep.

Ineffectiveness in training may be due to insufficient stimulation energy-either because the energy level is too low to induce an appropriate muscular response or because it is low enough that the patient can consistently override the stimulation. Additionally, failure could arise from indications of patient harm or pain, as reported through feedback or detected by sensors monitoring physiological responses. Pain or discomfort may prevent the patient from fully engaging in the training or could signal that the stimulation parameters are inappropriate for their condition. Since the system only stimulates one hemidiaphragm, the patient retains voluntary control over their diaphragm, unlike a pacemaker that directly controls cardiac activity. This means that the system supports, rather than fully dictates, diaphragm function, and the training's success depends on appropriately balancing the stimulation to achieve the desired respiratory improvements while avoiding harm or discomfort.

Table 2, below, shows examples of different operative mode combinations and stimulation profile parameters for respiratory conditioning, and the interplay between sleep mode and awake mode.

After the adjustments to the stimulation profile(s) are made, the process 250 then comes to an end.

FIG. 2C shows a detailed process of the patient control mode of the system of FIG. 1 in accordance with illustrative embodiments. For example, the processor 50 may perform steps of the process 200. FIG. 2C may have many or all of the steps of FIG. 2B. Furthermore, the detailed process of FIG. 2C may be integrated into FIG. 2B. For example, steps 258-266 (identified collectively as 290) of FIG. 2C may begin after step 257 of FIG. 2B and end before step 268 of FIG. 2B. Because of the similarity of steps with FIG. 2B, discussion of identical or similar steps is not again repeated here. After selecting the stimulation profile for respiration conditioning at the step 257, the process proceeds to step 258, which begins stimulation according to the stimulation profile.

TABLE 3

| | Min | Max | | | | | Rising Ramp/ |
|---|---|---|---|---|---|---|---|
| | Current | Current | Pulse | | | I:E | Falling Ramp/ |
| | Amplitude | Amplitude | width | Frequency | Rate | (duty | Baseline Current |
| | (mA) | (mA) | (μsec) | (Hz) | (bpm) | cycle) | Amplitude (%) |
| Example 1 | 5 | 9 | 300 | 40 | 15 | 1.0 | 20/20/50 |
| Example 2 | 3 | 7 | 150 | 20 | 12 | 0.8 | 30/10/50 |
| Example 3 | 1 | 3 | 60 | 10 | 10 | 1.0 | 30/30/50 |
| Example 4 | 5 | 9 | 150 | 20 | Variable | 1.2 | 20/20/30 |
| Example 5 | 5 | 9 | 300 | 40 | 15 | 0.5 | 10/10/70 |
| Example 6 | 5 | 9 | 300 | 40 | 10 | 1 | 50/50/50 |

Table 3 outlines daytime respiratory stimulation profiles designed to support various goals of respiratory therapy, such as rate training, depth of breath, diaphragm strengthening, and acclimation to breath pacing. Each example reflects a distinct stimulation profile, shaped by its specific stimulation parameters: amplitude (min and max), pulse width, frequency, breathing rate, I:E ratio, and ramping pattern.

The I:E ratio refers to the ratio of stimulation pulse train duration (inhalation phase) to the time between stimulation pulses (exhalation phase). A higher I:E value reflects a longer duration of stimulation relative to the rest period, which promotes prolonged inhalation or even a breath hold. Conversely, a lower I:E means shorter inhalation relative to exhalation. Additionally, the baseline amplitude percentage refers not to a percentage of the maximum amplitude shown in the third column of Table 3, but to a percentage of the maximum amplitude within an individual stimulation pulse train, providing more granular control of comfort and intensity. This baseline often begins at lower values to allow patient comfort before ramping up to stronger stimulation. In contrast, the maximum and minimum amplitudes shown in the second and third columns of Table 3 refer to the minimum and maximum currents over an entire stimulation profile made up of a series of individual stimulation pulse trains.

To further clarify, the baseline amplitude (%) represents the starting intensity of each stimulation burst, expressed as a percentage of the peak amplitude within that specific burst. It serves as the “floor” or initial level of stimulation from which the system ramps up during inhalation. This baseline is the starting point for each pulse cycle relative to that same cycle's peak.

The rising ramp (%) defines how quickly the stimulation increases from the baseline amplitude to its peak during inhalation. Rather than representing a portion of the overall power, this value functions more like a slope or speed setting. A smaller percentage indicates a steeper or faster rise to peak intensity, whereas a larger percentage creates a slower, more gradual increase.

Similarly, the falling ramp (%) controls how quickly the stimulation drops from the peak back down to the baseline during exhalation. Like the rising ramp, it doesn't represent a share of total amplitude but instead shapes the smoothness or sharpness of the decline in stimulation. Together, these parameters define the dynamics of each breath cycle and how the patient experiences the onset and offset of stimulation.

Stimulation Profile Example 1 uses a high-energy profile (5-9 mA, 300 μs, 40 Hz) with an I:E of 1.0 and balanced ramping (20/20/50), targeting depth of breath training. It is configured to train patients to breathe more deeply.

Stimulation Profile Example 2, by contrast, uses moderate energy levels (3-7 mA) and a shorter pulse width (150 μs) to help patients acclimate to a target breathing rate (12 bpm). Its I:E of 0.8 favors exhalation, and its ramp pattern (30/10/50) supports a fast rise in stimulation with a short fall-reinforcing a quicker inhalation.

Stimulation Profile Example 3 is a low-energy sensory prompting profile (1-3 mA, 60 μs, 10 Hz) with an I:E of 1.0, aimed at providing gentle sensory input to cue voluntary breathing rather than actively causing diaphragmatic contraction. The stimulation is configured to produce a sensation that prompts the patient to breath This suits patients undergoing sensation acclimation or early-stage conditioning.

Stimulation Profile Example 4 is a dynamic training profile that may be used to exercise the diaphragm and acclimate the patient. It starts at the patient's intrinsic breathing rate and intrinsic tidal volume, then gradually increases energy to boost tidal volume while slowing the rate (I:E 1.2), before ramping back down to the intrinsic rate and intrinsic tidal volume. The moderate energy and 20/20/30 ramp pattern allow for gradual transitions in stimulation intensity and timing.

Stimulation Profile Example 5 is configured for inspiratory hold training—with a short, forceful inhalation followed by a prolonged exhalation. Its I:E of 0.5 and ramp pattern of Oct. 10, 1970 emphasize the long exhale phase, which may support CO₂ clearance and diaphragm relaxation.

Stimulation Profile Example 6 encourages slow, deep breathing with both long inhalation and exhalation (I:E 1.0), high energy, and a fully balanced ramp pattern (50/50/50), promoting full respiratory engagement without a breath hold.

Across these examples, lower amplitudes and longer I:E values can be used for patient comfort and to support longer inhalation holds, which help deepen air intake and promote respiratory control. Likewise, decreased ramp durations help the stimulation reach peak intensity more quickly, reinforcing the inhalation phase and its associated respiratory training goals.

It should be understood that these stimulation profiles for awake time respiration conditioning are merely exemplary, and are not intended to limit various embodiments. At step 260 and while the IPG 12 provides stimulation to the patient, the patient-specific mobile device 215 may provide coordinated breathing instructions for the patient at a user interface (e.g., the user interface 220 described herein) of the patient-specific mobile device 215. Such breathing instructions may include for example, but not limiting of the disclosure:

### Session 1: Slow Deep Breathing (Visual or Audible)

Mode: Guided with Phrenic Nerve Stimulation
Instructions
   1. Get Comfortable.
      Sit upright or recline slightly. Relax your shoulders and jaw.
   2. Breath with the Stimulator
      Inhale gently through your nose or mouth
         Feel the stimulation begin.
      Exhale slowly when the stimulation stops
         Let your breath fall away naturally.
   3. Match the Rhythm
      Try to keep your breaths in sync with the stimulation.
      Allow the pulse to guide your breathing pace.
   4. Focus on the Sensation
      Feel your belly rise as you inhale.
      Let your body relax as you exhale.
   5. Additional Notes
      If you lose sync, simply wait for the next stimulation and begin again.
      If using audio or vibration, follow the cue with your breath.
         For a tactile instruction, for example, the patient can be instructed to start their inhale when they feel vibration or pulsing, and to begin their exhale when the vibration or pulsing stops. Additionally, or alternatively, the breathing instruction may show visual representation of a breathing instruction (e.g., with target breathing pattern.

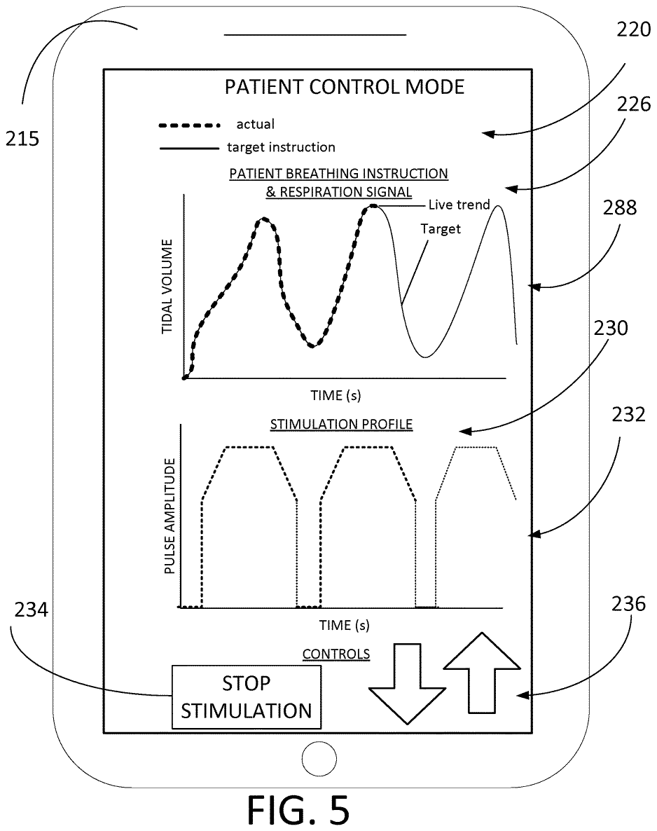

FIG. 5 schematically shows the user interface 220 of the patient control mode in accordance with illustrative embodiments described herein. The user interface 220 is configured to provide an intuitive and interactive experience for the patient. The interface 220 includes a dynamic visual representation 288 of the breathing instruction 226. As shown, the visual representation 288 may include a waveform depicting the target breathing pattern. This target waveform may be displayed overlayed with or alongside a real-time graph of the patient's actual breathing (e.g., the live trend), allowing the patient to compare their performance with the target. Furthermore, the associated patient breathing instruction may be temporally aligned with the selected stimulation profile 230. The interface 220 may also display a dynamic visual representation 232 of the stimulation profile 230 being delivered by the phrenic nerve stimulator. Patient stimulation occurs in accordance with the predefined stimulation profile, which may include parameters such as intensity, frequency, and duration of the electrical impulses. These parameters may be displayed on the user interface, helping the patient understand the structure and purpose of the stimulation as it synchronizes with the respiratory conditioning regimen.

In some embodiments, the dynamic visual representations 288 and 232 scroll continuously in real time, displaying the most recent data (e.g., live trend data and/or the target data), while older data scrolls off the screen. Alternatively, in some embodiments the dynamic trend line 288 may refresh after a fixed duration (e.g., 10 seconds), clearing the previous data and restarting from the origin.

Various embodiments may include a compliance indicator (not shown) that provides real-time feedback on the patient's adherence to the breathing instruction, using visual cues such as a percentage score, color-coded indicators (e.g., green for high compliance), or encouraging messages. This feature motivates the patient to align their breathing efforts with the target pattern. In some embodiments, the feedback from the device to the patient may be based on the synchrony between the patient's breathing and the stimulation pattern. Based on this analysis, the system may provide dynamic coaching prompts—such as “slow down,” “speed up,” “take deeper breaths,” or “good job, you're right on target.” In place of, or in addition to, a graphical display of the stimulation profile, simple animations may be used to guide and reinforce proper breathing, such as a balloon gently expanding and contracting in sync with the desired pattern.

Control buttons 236 on the interface allow the patient to personalize their experience, including options to adjust the stimulation profile within safe, predefined limits or to request modifications to the breathing instruction. A prominent “Deactivate” button 234 is accessible, enabling the patient to stop the stimulation immediately. By selecting the deactivate button 234, a deactivation signal is sent to the stimulator. The user interface is configured to display data and controls that help the patient confidently engage with the respiratory conditioning session, improving patient confidence and compliance during the respiratory conditioning session.

After stimulation begins, at step 261 the process determines whether the stimulation creates patient discomfort. This determination may be made by a user input. For example, the patient may select the deactivate button 234, or may request that the stimulation profile intensity be lowered using the control buttons 236. Accordingly, the patient may provide a stimulation profile adjustment signal to the system.

Additionally, or alternatively, patient feedback may be obtained via one or more sensors. For example, pain and discomfort indicators, such as skin conductance sensors (GSR) may measure changes in skin conductivity linked to stress or discomfort, while heart rate monitors and respiratory rate sensors detect physiological responses like rapid heartbeats or irregular breathing patterns, which may signify pain or strain. Additionally, electromyography (EMG) sensors can measure involuntary muscle activity, such as spasms near the stimulation site, that indicate discomfort. Strain or difficulty indicators, including oxygen saturation monitors (such as a pulse oximeter), assess whether the patient is experiencing impaired breathing or overexertion, and motion sensors can detect sudden or abnormal movements that suggest difficulty. These measurements are analyzed in real-time to identify signs of overstimulation, such as significant increases in stress-related biomarkers, and/or irregular breathing patterns.

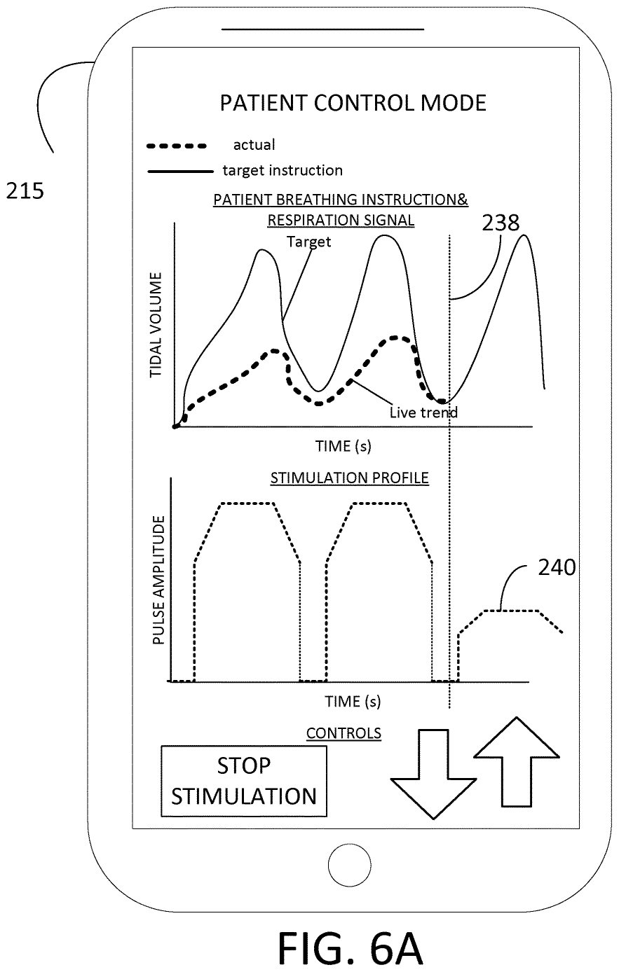

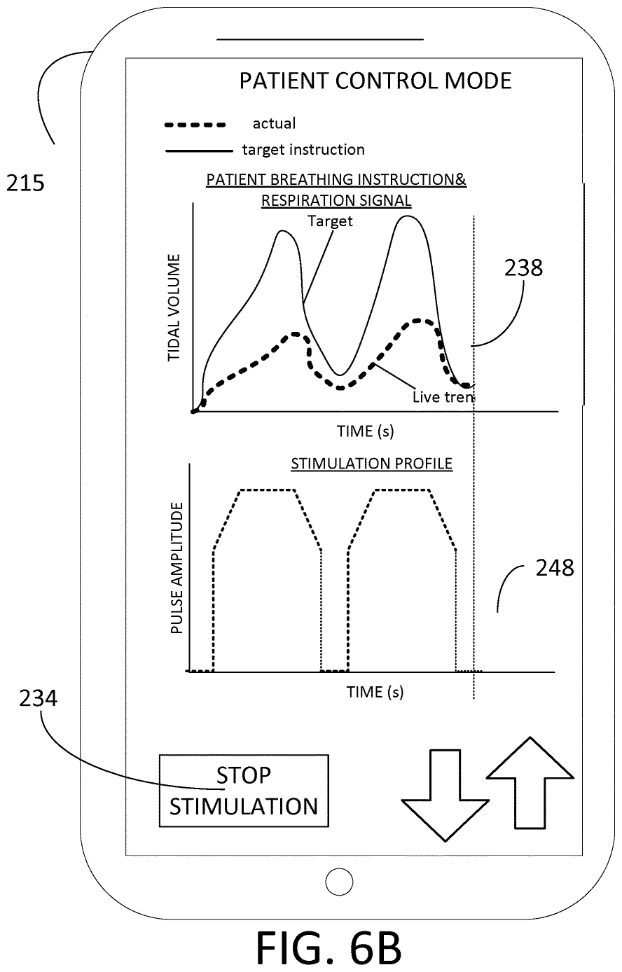

If the patient indicates that the stimulation is too difficult or painful and/or a pain threshold indicating discomfort or strain is reached, the process proceeds to step 262, which adjusts the respiratory conditioning stimulation profile or prematurely ends the stimulation. FIG. 6A schematically shows the user interface 220 of the patient control mode when the stimulation profile is adjusted in accordance with illustrative embodiments described herein. As shown, the stimulation profile is adjusted at a time 238 when the patient uses the controls to lower the stimulation or when the system detects signs of distress. If the patient presses the control button to decrease stimulation, the system adjusts the intensity, frequency, or duration of the stimulation profile to ensure a more comfortable experience. Simultaneously, the display updates to reflect these changes in real time. Accordingly, an adjusted stimulation profile 60(B) may be displayed.

Additionally, or alternatively, the device continuously monitors biofeedback indicators, such as irregular breathing patterns, rapid breathing, or other signs of stress detected through sensors (e.g., respiratory rate monitors, skin conductance sensors, fluid load sensors, or heart rate monitors). When these signs suggest that the patient is struggling to breathe or experiencing discomfort, the system automatically modifies the stimulation profile to alleviate strain. In some embodiments, the signal from the fluid load sensor may be used to calibrate a transthoracic impedance value from the transthoracic impedance signal to the tidal volume of the patient based on the fluid load of the patient.

In a similar manner, the system may stop stimulation altogether. FIG. 6B schematically shows the user interface 220 of the patient control mode when the stimulation is stopped in accordance with illustrative embodiments described herein. In various embodiments, the system is configured to prioritize patient safety and comfort by immediately stopping the stimulation altogether if the patient indicates distress or if the device detects significant physiological or behavioral signs of discomfort. If the patient presses the dedicated “Stop” button 234 on the user interface at time 238, a deactivation signal is sent and the system halts all stimulation, ensuring no further discomfort. This action may be reflected on the display, which indicate that the stimulation has been stopped and prompts the patient to rest or take further action if needed.

The process then returns to step 256, which waits to receive the patient session activation signal (e.g., if the stimulation was stopped). Alternatively, if the stimulation profile was adjusted in real-time, the process may proceed directly to step 258 and continue stimulation.

If the stimulation is not too painful, the process proceeds to step 264, which monitors a patient respiration signal and other objective biofeedback. Thus, the system may continuously monitor biofeedback indicators, such as abnormal breathing patterns, excessive respiratory rate, or signs of stress detected through sensors (e.g., heart rate spikes, irregular muscle activity, or skin conductance changes).

At step 266, if these indicators suggest that the patient is struggling significantly to comply with the breathing instructions, or is at risk at time 238, the process returns to step 262 and prematurely ends or adjusts the stimulation to prevent overstimulation or harm. In some embodiments, this determination may be made by the processor tracking the one or more respiratory parameters from step 264 of the patient over successive one or more respiration conditioning sessions, and determining a difference between the one or more respiratory parameters and the respiratory goal. Furthermore, the processor may store the one or more respiratory parameters, the difference, and an associated time stamp in a memory associated with the IPG. The at least one processor may store one or more stimulation parameters that are associated with an achievement of the respiratory goal, based on the difference between the one or more respiratory parameters and the respiratory goal being substantially zero.

The user interface 220 then updates to confirm that stimulation has ceased (e.g., stopped stimulation profile 248). This safety feature ensures that the system responds promptly to protect the patient during respiratory conditioning sessions.

If the patient is above a pre-set threshold compliance with the breathing instructions, the process continues to step 268 where the respiration conditioning session is completed. Such compliance may include for example, parameters such as the percentage of breaths that are synchronized with the stimulation pattern, consistency of inhale/exhale timing with tactile or visual cues, breath depth (e.g., based on tidal volume proxies or respiratory effort sensing), and overall synchronization rate over a defined time window. In some embodiments, compliance may further consider whether the patient successfully completes the specified profile duration or number of repetitions, particularly in profiles where stimulation amplitude and rate are varied. Failure to synchronize for the full profile duration, or to maintain synchrony across changing stimulation parameters, may indicate non-compliance. Additionally, compliance evaluation may account for parameters beyond the current session, for example, whether the patient has met prescribed training frequency targets—such as completing a required number of sessions per day or on designated days. These metrics, obtained through synchronization detection or other respiratory sensing capabilities, may be integrated into a patient compliance score to determine whether the patient is eligible to advance to subsequent training sessions.

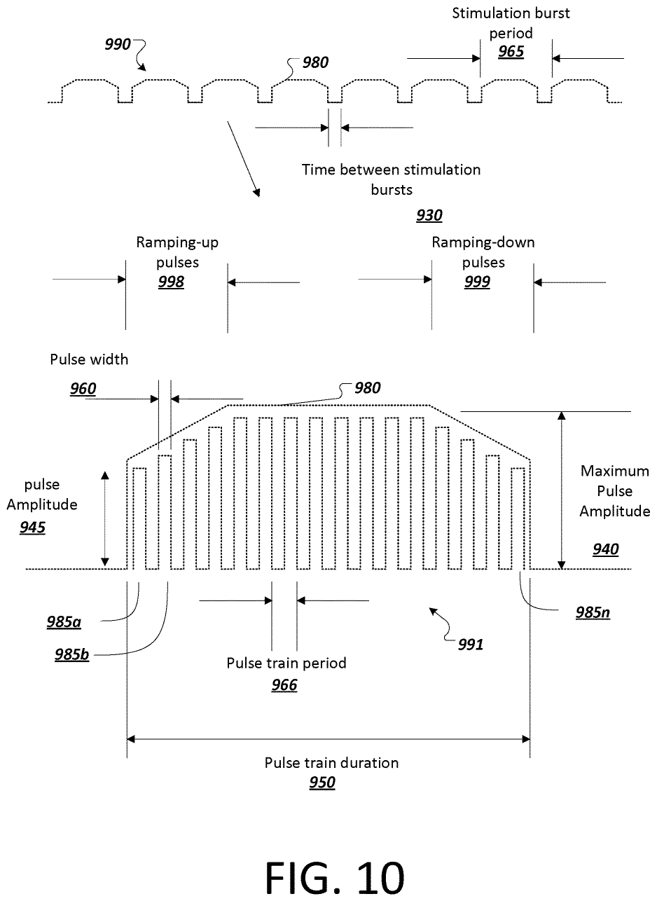

FIG. 10 illustrates an example of an electrical stimulation trace 990. The electrical stimulation includes a series of stimulation bursts 980. Each stimulation burst 980 may be provided as a pulse train 991, such that the stimulation burst 980 is a pulse envelope for the individual pulses (for example, the n pulses 985a, 985b, . . . , 985n) of the pulse train 991. The pulse train 991 provides a burst of individual pulses. The leads 14, 16 may each include electrodes to deliver electrical stimulation to a target nerve according to the electrical stimulation profile 58 that specifies the various parameters or characteristics illustrated for the electrical stimulation trace 990. For example, the processor 50 may control the stimulation circuitry 42 to provide the electrical stimulation bursts to the leads 14, 16. The stimulation bursts 980 are delivered at a stimulation burst period corresponding to a stimulation rate. The stimulation rate (for example, the rate of the stimulation bursts 980) may be within the range of about 0.1 Hz to about 0.5 Hz, corresponding to a targeted stimulated breathing rate of 6 to 18 breaths per minute. In some examples, a stimulation rate indicated by a stimulation profile may be determined based on a predetermined unstimulated respiration rate, that is, a predetermined respiration rate when stimulation is not applied.

For example, the stimulation rate may be equal to the predetermined unstimulated respiration rate, or may be greater or less than the predetermined unstimulated respiration rate. In one example, the stimulation rate may be a predetermined number of cycles-per-minute fewer than the predetermined unstimulated respiration rate (for example, one cycle-per-minute fewer, two cycles-per-minute fewer, three cycles-per-minute fewer, or some other value). In other examples, the stimulation rate is a predetermined number of cycles-per-minute greater than the predetermined unstimulated respiration rate (for example, one cycle-per-minute greater, two cycles-per-minute greater, three cycles-per-minute greater, or some other value). A stimulation burst 980 may be 1-10 seconds in total duration. In the example of FIG. 10, the total duration of the stimulation burst 980 is the pulse train duration 950. For example, the stimulation burst may be about 2 seconds in total duration. Each stimulation burst 980 may be provided as a pulse train having a pulse train period 966 corresponding to a pulse train frequency. The pulse train frequency (that is, the frequency of the individual pulses within the stimulation burst 980) may be 10-40 Hz.

The electrical stimulation profile 58 may specify parameters including one or more of the stimulation rate (corresponding to the stimulation burst period 965), the pulse train duration 950, and the pulse train frequency (corresponding to the pulse train period 966). Additionally, the parameters may include a time between stimulation bursts 930. The pulse train envelope forming each stimulation burst 980 defines the maximum pulse amplitude 940 as the maximum amplitude of one or more individual pulses within the pulse train. Each individual pulse may be further characterized by a pulse width 960. As illustrated in FIG. 10, the individual pulses 985a, 985b, . . . , 985n of the pulse train may not all have the same pulse amplitude 945 and may vary in amplitude so as to ramp up 998 the energy of the stimulation burst 980 and/or ramp down 999 the energy of the stimulation burst 980. The ramp up may occur over a ramping-up time, for example, the time interval for the ramping up pulses 998. The ramp down may occur over a ramping-down time, for example, the time interval for the ramping down pulses 999. In some examples, the stimulation burst 980 may not include a ramp up and/or a ramp down. The IPG 12 configuration with regard to operational parameters and/or with regard to stimulation circuitry 42 may exclude a square wave capability for an electrical stimulation profile in which the pulse trains exclude both the ramping-up period and the ramping-down period.

The electrical stimulation delivered by the IPG 12 may be characterized by one or more electrical stimulation parameters as specified by the electrical stimulation profile 58. The IPG 12 may adjust one or more of these parameters to titrate the electrical stimulation energy delivered to a target nerve. The electrical stimulation parameters may include pulse train parameters. The pulse train parameters, as discussed above, may include one or more of a pulse amplitude 945, a maximum pulse amplitude 940, a pulse train period (or frequency) 966, a pulse width 960, and/or a pulse train duration 950. The pulse amplitude 945 may include an amplitude of at least one of the ramping-up pulses, an amplitude of the constant-amplitude pulses, and/or an amplitude of at least one of the ramping-down pulses. The pulse train parameters may further include one or more of a time over which an amplitude of the pulses is ramped up, a time over which the amplitude of the pulses is ramped down, a number of ramping-up pulses, and/or a number of ramping-down pulses. The stimulation parameters may further include one or more of the stimulation rate corresponding to the stimulation burst period 965, the time between stimulation bursts 930, a stimulation current, a stimulation voltage, and a stimulation polarity (for example, monophasic or biphasic), stimulation energy ramps, and so forth. The stimulation energy ramps may include changes in energy delivered by a stimulation burst 980 over a series of stimulation bursts 980. One or more electrical stimulation parameters may be adjusted to change the energy delivered by stimulation bursts 980 over time to titrate the stimulation energy delivered to the target nerve. Such titration may achieve a desirable balance between patient comfort and stimulation efficacy. For example, more intense electrical stimulation of a patient's phrenic nerve may yield higher activation of a patient's diaphragm, which may increase the efficacy of nerve stimulation therapy in some examples. The stimulation profile 58 may also include titration parameters for titration of stimulation energy over a period of minutes, hours, days, months, and so forth.

However, more intense electrical stimulation may also yield a greater sensory response from a patient. For example, a patient may feel more discomfort as the stimulation intensity is increased. Experiencing discomfort may adversely impact a patient's sleep. For example, even if a particular set of stimulation parameters effectively activate the patient's diaphragm, treatment according to that particular set of stimulation parameters may be contraindicated if the particular set of stimulation parameters causes the patient to wake up and lose sleeping time, for example due to discomfort produced by the particular set of stimulation parameters. It is therefore beneficial to the efficacy of stimulation therapy to identify stimulation parameters that produce a desired muscle response (for example, diaphragm contraction and expansion) while minimizing or avoiding patient discomfort.

In an implementation, the IPG 12 may ramp up the stimulation amplitude to increase the intensity of the stimulation, or may ramp down the stimulation amplitude to decrease the intensity of the stimulation. In general, the stimulation amplitude may vary over a range from a minimum amplitude for a pulse train to a maximum amplitude for the pulse train. The individual pulses within the pulse train may vary in amplitude but the maximum amplitude defines the maximum amplitude reached during a stimulation pulse train. In at least one example, individual pulse train pulses that constitute a stimulation burst may ramp up for a period of time and may ramp down for a period of time. In various examples, a start time of a pulse train may refer to a time at which a pulse train begins to ramp up, and an end time of a pulse train may refer to a time at which a pulse train finishes ramping down.

The one or more parameters may include one or more of a duration of a ramping-up period and/or an amount by which to increase the energy during the ramping-up period, a duration of a ramping-down period and/or an amount by which to decrease the energy during the ramping-down period, a duration of a time between ramping-up and/or -down periods during which energy is maintained, a pulse amplitude, a number of pulses in a pulse train, a pulse width of the pulses, a time between pulse trains, a waveform of each pulse, a frequency of pulses, a pulse amplitude, a maximum and/or minimum pulse amplitude, a stimulation current, a stimulation voltage, a stimulation polarity (for example, monophasic or biphasic), or any other parameters affecting how the IPG 12 delivers the stimulation energy to the patient.

Adjustment of stimulation parameters to adjust stimulation intensity may include a change in one or more stimulation profile parameters. The adjustment of the stimulation intensity may be referred to as a titration. An increase in stimulation energy may resolve ineffective stimulation, or stimulation that fails to effectively change or resolve irregularities in respiration. For example, adjustment or titration may include increasing or decreasing one or more of current amplitude (e.g., the pulse amplitudes 940 and/or 945 as shown in FIG. 10), pulse width (e.g., the pulse width 960 as shown in FIG. 10), pulse train period or frequency (e.g., the pulse train period 966 as shown in FIG. 10). As additional examples, adjustment or titration may include increasing or decreasing a pulse train duration (e.g., the pulse train duration 950 which is the duration of the stimulation burst 980) and/or shortening, lengthening, or changing the slope of the rising and/or falling ramps, etc. to name a few further examples. As further examples, adjustment or titration may include increasing or decreasing an intensity of electrical stimulation in response to the respiration reduction. The intensity titration may include titration of stimulation parameters (e.g., current, amplitude, pulse width, pulse train frequency, etc.). For example, a pulse train frequency of 40 Hz is more intense than a pulse train frequency of 20 Hz. As an additional example, the adjustment may include a change to the inspiration to expiration ratio (I:E).

FIG. 11A schematically shows details of a neurostimulation system 5 in accordance with illustrative embodiments described herein and FIG. 11B schematically shows details of a processor and memory of the IPG 12 in the system 5. Each of these components is operatively connected by any conventional interconnect mechanism.

FIGS. 11A and/or 11B simply shows a bus communicating each the components. Those skilled in the art should understand that this generalized representation can be modified to include other conventional direct or indirect connections. Accordingly, discussion of a bus is not intended to limit various embodiments.

Indeed, it should be noted that FIGS. 11A and 11B only schematically shows each of these components. Those skilled in the art should understand that each of these components can be implemented in a variety of conventional manners, such as by using hardware, software, or a combination of hardware and software, across one or more other functional components. For example, patient control mode controller 152 may be implemented using a plurality of microprocessors executing firmware. As another example, the patient control mode controller 152 may be implemented using one or more application specific integrated circuits (i.e., “ASICs”) and related software, or a combination of ASICs, discrete electronic components (e.g., integrated circuits), and microprocessors. Accordingly, the representation of the patient control mode controller 152 and other components in a single box of FIG. 11A or 11B is for simplicity purposes only. In fact, in some embodiments, the controller of FIG. 11A or 11B is distributed across a plurality of different components—not necessarily within the same housing or chassis. For example, one or both of the profile libraries 57A and 57B can additionally or alternatively be on the patient-specific mobile device 215.

The respiratory conditioning system 5, as discussed herein, includes an implantable system 10 and an external computing device 210, and one or more external sensors 18. The implantable system 10 includes an implantable medical device 12 (for example, an implantable pulse generator 12, or IPG 12), one or more phrenic nerve stimulation leads 14, one or more airway nerve stimulation leads 16, and, optionally, one or more optional additional leads 13 and/or one or more optional distributed sensors 22 and/or one or more external sensors 18. The implantable system 10 may be configured to be implanted in a patient. For example, FIG. 1 illustrates a patient 20 implanted with the implantable system 10 according to an example.

The system 5 may include sensors. The sensor(s) may provide sensed information to the IPG 12, such as therapy-specific indicator data and/or patient-specific indicator data. The sensors may be external sensors 18, implantable IPG sensors 19, and/or implantable distributed sensors 22. The sensor(s) 18 (for example, at least one first sensor and at least one second sensor) may include sensors implanted inside of the patient and/or sensors disposed externally on or near the patient. One or more sensors may be disposed on an external surface of the patient. In some embodiments, the leads may be separate from the sensor(s) 18. Additionally, or alternatively, the leads may include or be coupled to one or more of the sensor(s) 18.

Among other things, the sensors 18 may include a belt-style sensor, such as a respiratory inductance plethysmography belt. The belt-style sensor 18 may also include an accelerometer-based belt. Additionally, or alternatively, the sensors 18 may include finger sensors, such as pulse oximeters and/or photoplethysmography (PPG) sensors.

In an implementation, the sensors 19, 32a, 32b, 22, and/or 18 may include at least one cardiac sensor (e.g., a heart rate sensor and/or an electrocardiogram (ECG) sensor). The cardiac sensor(s) may enable the IPG 12 to monitor heart rate variability as a function of patient position and/or monitor the heart rate, sinus rhythm, ECG signal and correlate those measurements with AF targeted therapy and/or incidental AF benefits from sleep apnea targeted therapy. In an implementation, the cardiac sensor(s) may include a transthoracic impedance sensor, or an ECG sensor configured to measure an electrical vector across the patient's chest due to heart beat (e.g., measurements of electrical signal changes due to cardiac tissue contractions). In an implementation, the cardiac sensor(s) may include a dedicated lead proximate to and/or connected to heart tissue. The IPG 12 may monitor the cardiac signals intermittently or periodically or on a substantially continuous basis.

The IPG 12 may include IPG sensors 19, principal stimulation circuitry 42, subordinate stimulation circuitry, at least one processor 50, a wireless communication interface 52, at least one power supply 54, and a memory 56.

As shown in FIG. 11B, the processor 50 may include a clock 29 and is configured to include and/or execute a patient control mode controller 152, a sleep mode controller 154, an operational mode controller 156, a patient therapy mode controller 6, and a stimulation profile adjuster 158. The clock 29 may count time over a lifetime of the processor 50 and may correlate to real time of day and date. The patient control mode controller 152, the sleep mode controller 154, the operational mode controller 156, the patient therapy mode controller 199, and the stimulation profile adjuster 158 may include hardware (for example, one or more controllers configured to execute computer-executable instructions and/or one or more hard-wired circuits), software and/or firmware (for example, instructions stored on non-transitory computer-readable media that, when executed by hardware such as the one or more controllers, causes the system 10 to deliver therapy), or a combination thereof. In some examples, the actions performed by non-sleep time period mode controller 152 (or a patient control mode controller 152), sleep mode controller 154, operational mode controller 156, patient therapy mode controller 199, and stimulation profile adjuster 158 may be implemented by hardware, software, firmware, microcode, hardware description languages, and so forth, or any combination thereof. When implemented in software, firmware, or microcode, the program code or code segments to perform the tasks may be stored in a non-transitory processor-readable medium such as a storage medium. One or more processors, including the processor 50, may perform the described tasks. In an implementation, the at least one processor may include one or more integrated circuits and/or at least one application specific integrated circuit (ASIC).

An operational mode controller 156 determines an operational mode of the system 10. As shown in FIG. 3, the operational mode controller 156 determines whether the current time is within a pre-determined sleep time period. If yes, the device enters a sleep mode, where stimulations are controlled by the sleep mode controller 154. Alternatively, the device may be in a null mode, where it is not active and stimulation is deactivated. The operational mode controller 156 also determines whether the current time is within a pre-determined non-sleep time period. If yes, the device enters a patient control mode, where stimulations are controlled by the patient control mode controller 152. The operational mode controller 156 is further configured to allow selection between modes, ensuring that only one mode is active at any given time. For instance, the device may transition between the patient control mode and the sleep mode based on a programmed schedule, manual input, or analysis of physiological data (e.g., respiration rate or time of day). This ensures that the device delivers stimulation appropriate for the patient's activity level or state of rest.

The device configured to provide this capability through selectable modes, where the processor and control logic are configured to deliver only one stimulation pattern at a time in accordance with the active mode.

The patient control mode controller 152, or equivalently the non-sleep time conditioning mode controller 152, includes a respiration conditioning session scheduler 151 and a conditioning stimulation profile selector 153. Based on patient data (e.g., weight, condition, sex, etc.) and/or medical practitioner entered input, the scheduler 151 produces a respiration conditioning schedule for a patient awake time period. The schedule includes one or more stimulation profiles 58 (e.g., selected by the selector 153) and spread out over the course of the awake time period.

The patient control mode controller 152 has a UI module 163 that is configured to communicate with the external devices (e.g., patient-specific mobile device 215, sensors 18, external computing device 210, etc.) via the communications interface 52. The UI module 163 allows the controller 152 to receive patient feedback directly from the patient or sensors 18. This information may be sent to a profile adjuster 158 configured to make changes to the profiles and/or select different profiles. The stimulation profile adjuster 158 includes a conditioning stimulation profile adjuster 158A for adjusting respiratory conditioning stimulation profiles 58, and a sleep stimulation profile adjuster 158B for adjusting sleep stimulation profiles 60. The conditioning stimulation profile adjuster 158A may additionally be configured to make adjustments to the corresponding instructions 59 for the stimulation profile. Additionally, a patient preference module 161 receives input from patient controls (e.g., see FIG. 5), providing a patient preference regarding the stimulation (e.g., the intensity). An activation signal checker 165 checks for the receipt of an activation signal from the patient before send instructions to the stimulation circuitry to begin the stimulation session. Similarly, a deactivation signal checker 167 monitors for the receipt of a deactivation signal that sends instructions to the stimulation circuitry to immediately end the stimulation session.

During this awake-time window, the IPG operates in a patient-controlled mode, meaning the patient can start, stop, and/or adjust the stimulation delivery as needed. For instance, stimulation may begin when the patient activates the session via a smartphone interface or external controller and may stop either at the end of the scheduled session or when the patient manually deactivates the therapy. This setup provides flexibility and ensures that stimulation aligns with the patient's comfort and readiness, while also allowing for real-time adjustments based on patient feedback or detected physiological responses.

Post-stimulation feedback may include both respiratory and non-respiratory information, such as respiratory patterns, patient activity levels, body position, heart rate, and sleep stage. For instance, higher activity levels outside the therapy window can indicate more effective therapy, as the patient is likely to feel more energetic and less lethargic. Additionally, feedback may include patient-reported information, such as survey responses about sleep quality, respiration conditioning energy levels, and other health indicators linked to reduced sleep disruptions.

This post-therapy information, along with therapy-effectiveness data collected during the therapy window, can be used to optimize treatment. For example, both the patient and the clinician may provide feedback to the system or a connected computing platform to fine-tune therapy settings. Adjustments could be based on a combination of objective data, like heart rate and sleep metrics, and subjective assessments, such as improved quality of life and reduced respiration conditioning fatigue. This integration of feedback ensures a personalized and adaptive approach to therapy.

The sleep mode controller 154 controls selection of sleep stimulation profiles 59 during the pre-determined sleep time period. In general, these sleep stimulation profiles 59 are configured to treat sleep apnea. The sleep mode controller 154 may include an entrainment engine 24 and an entrainment diagnostics engine 28. In an implementation, the external computing device 210 may also include the entrainment engine 24. The entrainment engine 24 may include hardware (for example, one or more controllers configured to execute computer-executable instructions), software and/or firmware (for example, instructions stored on non-transitory computer-readable media that, when executed by hardware such as the one or more controllers, causes the system 10 to deliver therapy), or a combination thereof. In some examples, the actions performed by the entrainment engine 24 may be implemented by hardware, software, firmware, microcode, hardware description languages, and so forth, or any combination thereof. When implemented in software, firmware, or microcode, the program code or code segments to perform the tasks may be stored in a non-transitory processor-readable medium such as a storage medium. One or more processors, including the processor 50, may perform the described tasks.

In an implementation, the patient therapy mode controller 199 controls the sleep stimulation profile selector 157 and the conditioning stimulation profile selector 153 based on a stored patient therapy mode indication 62, for example in the patient data 61 of the memory 56. The patient therapy mode indication 62 may be a pre-programmed patient therapy mode for sleep apnea therapy, AF therapy, or a combination thereof, e.g., from the external computing device 210, or may be a patient input therapy request from the patient specific mobile device 215.

The processor 50 may also include the clock 29. The clock 29 may be configured to identify a time of day, day of the week, month of the year, or another time indicator or clock information. In an implementation, the IPG 12 may use the clock information to control delivery of electrical stimulation signals to the leads 14 and/or 16.

The system includes one or more stimulation profile libraries 57. In particular, the system may have a dedicated library 57A for respiratory conditioning stimulation profiles 58, and corresponding breathing instructions 59. Some embodiments may also include a sleep stimulation profile library 57B having sleep stimulation profiles 60. One of skill in the art will appreciate that sleep stimulation profiles 60 generally do not have corresponding patient breathing instructions 58, as the patient is asleep.

As described previously, each profile 58, 60 is defined by a set of electrical stimulation parameters, including among other things, current amplitude, which determines the intensity of the pulses (e.g., 2 mA to 10 mA); frequency, which sets the number of pulses per second (e.g., 20 Hz for rapid stimulation or 10 Hz for slower breathing); pulse width, which controls the duration of each pulse (typically 150 μs to 300 μs); and stimulus morphology, which shapes the stimulation bursts with varying rise and fall times (ramps) and overall durations. These profiles 58, 60 are designed to achieve different therapeutic goals, such as promoting deep breathing with lower frequencies and longer ramps, improving vagal tone, improving overall cardiovascular health through decreases in adrenaline release, improved parasympathetic performance, etc., enhancing muscle strength with higher amplitudes, supporting relaxation with smoother, gradual stimulation patterns, and/or other physiological benefits of respiratory conditioning.

Each respiration conditioning stimulation profile 58 in the library 57A is paired with a corresponding breathing instruction 59, ensuring that the patient's active engagement aligns with the therapeutic intent. Instructions may guide the patient to breathe at a specific rate, such as 12 breaths per minute, emphasize deeper inhalations to expand lung capacity, or synchronize their breathing with the stimulation's rising and falling phases. These instructions are typically presented through a user interface, such as a smartphone app or external device, enabling the patient to participate actively in their respiratory conditioning.

The stimulation profiles 58 within the respiratory conditioning system may be provided in coordinated sets (e.g., 58A(i) . . . 58A(N)), each designed to progress systematically through a sequence of conditioning goals. For example, a coordinated set of stimulation profiles might begin with low-intensity stimulation to warm up the diaphragm, progress to higher amplitudes and longer ramp durations to promote deeper breathing, and conclude with a tapering profile to support relaxation.

One or more of the stimulation profiles 58 and 60 may correspond to a particular therapy mode of sleep apnea 242, AF 244, or AF+Sleep apnea 246, as determined at the step 240 in FIG. 2A-2 . . . . Furthermore, the coordinated breathing instructions 59 may also correspond to the particular therapy mode determined at the step 240 in FIG. 2A-2.

In various embodiments, the sleep mode controller 154 selects sleep stimulation profiles 60 as part of a therapy. The sleep mode controller 154 may communicate with the patient control mode controller 152 and/or the patient data database 61. The sleep mode controller 154 may access the patient results of the awake respiratory conditioning sessions (e.g., patient feedback, improved tidal volume, respiratory effort, patient compliance) and the corresponding stimulation profile 58. Based on this information, the sleep mode controller may adjust its selection of sleep stimulation profiles 60 for sleep therapy.

For example, if daytime conditioning has stimulations of increasing amplitude, the sleep mode controller 154 (e.g., the sleep stimulation profile selector 157) may select a sleep stimulation profile 60 with increased stimulation intensity or frequency because the patient is more likely to be accustomed to the sensation of stimulation. As another example, the sleep treatment therapy scheduler 155 may expedite the initiation of a sleep stimulation (e.g., after surgery) upon the patient exhibiting particular feedback (e.g., no pain) for a given conditioning stimulation profile 58. In some embodiments, the sleep mode controller 154 may recommend these actions to the patient and/or medical practitioner, and may seek an activation signal to change the therapeutic plan.

Additionally, as the patient demonstrates acclimation through successful completion of daytime stimulation profiles, the sleep mode controller 154 may adjust nighttime parameters such as shortening delays after patient movements (e.g., rolling or returning from a bathroom break) to increase effective therapy time. Sleep latency—the pause before beginning stimulation after detecting sleep—may also be reduced, allowing the therapy to initiate sooner once sleep is detected, further enhancing overall treatment efficiency.

A stimulation profile adjuster 158 may adjust the order of stimulation profiles within the coordinated set based on patient feedback and physiological responses. To that end, the adjuster 158 communicates with one or more sensors 18 and communications interface 52 to determine that a patient struggles with a specific profile 58A(i), such as one requiring higher amplitude or frequency. The adjuster 158 may adjust the stimulation sequence to provide additional conditioning with less challenging profiles (e.g., to 58B(i)) before reintroducing the more advanced settings. Similarly, patient-reported comfort levels or compliance data can guide the reordering of profiles to ensure that therapy remains effective and tolerable. This adaptive approach allows the system to deliver personalized therapy, improving patient engagement and maximizing the therapeutic benefit of the stimulation profiles.

The wireless communications interface 52 may include an electromagnetic telemetry transceiver along with at least one antenna for telemetry-based data communication and programming. Alternatively or additionally, the wireless communications interface 52 may include one or more of a transmitter, receiver, and/or transceiver configured for wireless communications, for example, according to a Bluetooth® protocol or other short-range communications protocol. In various implementations, the principal stimulation circuitry 42, the subordinate stimulation circuitry 44, and the at least one processor 50 may support dual-channel operations to support stimulation of a combination of at least one upper-airway nerve and at least one phrenic nerve.

The system 10 is configured to be communicatively coupled to the computing system 210 via a wireless communication connection 30. Wireless communications may be exchanged pursuant to a short-range wireless communication protocol such as, for example, the Bluetooth® wireless communication protocol, or inductive telemetry, or another telemetry method. The computing system 210 may program and/or configure the IPG 12.

The IPG 12 may receive programmable operating parameters via software and/or firmware downloaded to the IPG 12 via wired or wireless communications. In an implementation, the external computing device 210 may generate the programmable operating parameters, for example, based at least in part on user input to the external computing device 210. These programmable operating parameters may include one or more of stimulation energy and/or energy titration parameters.

It should be noted that discussion of conditioning stimulation profiles 58 also applies to sleep stimulation profiles 60, unless the context otherwise requires. Therefore, for convenience, reference to the stimulation profiles 58 applies to the conditioning stimulation profiles 58 and the sleep stimulation profiles 60. In a similar manner, reference to the stimulation profile library 57 applies to the stimulation profile library 57A and to the sleep stimulation profile library 57B. This discussion is therefore not repeated again for the sleep stimulation profiles 60 or the sleep stimulation profile library 57B.

In some examples, one or more stimulation profiles 58 may specify programmable operating parameters and/or the electrical stimulation parameters thereof, as discussed in greater detail elsewhere herein. The IPG 12 may store the one or more stimulation profiles 58 in a stimulation profile library 57. The one or more stimulation profiles may include one or more continuing stimulation profiles and/or one or more intermittent stimulation profiles. Additionally, the one or more stimulation profiles 58 may include stimulation profiles for the principal channel and/or for the subordinate channel. Each of these may be continuing stimulation or intermittent stimulation profiles depending on the particular stimulation mode selected by the operational mode controller 156. Over the course of therapy provided during a sleeping period of the patient, the sleep mode controller 154 may select a first, second, third, or additional sleep stimulation profile 60 for a particular channel, as discussed in further detail elsewhere herein. In a similar manner, the patient control mode controller 152 may select a first, second, third, or additional conditioning stimulation profile 58 for a particular channel, in addition to an associated patient breathing instruction 59. The selected stimulation profile may be retrieved from the library 57 (e.g., stored in the memory of the IPG 12). In an implementation, the patient control mode controller 152 may select the conditioning stimulation profiles 58 based on a patient therapy mode as determined at the step 240. For an IPG, various electrical circuitry components may be enclosed in a hermetically sealed casing to protect these components from the body environment. The IPG may further include a header disposed on the casing. The header may be a structural component of the IPG that provides one or more receptacles, or connector ports, configured to receive respective connector assemblies for leads connected to electrodes. Each lead may couple with one or more connector ports. The header thus provides paths to electrically and mechanically couple the leads to the enclosed components of the IPG 12 without comprising the hermetic seal. The connector ports may be configured and/or re-configured as sensor ports, stimulation ports, sensor-and-stimulation ports, and so forth according to programmed configuration settings provided to the IPG 12 through the firmware and/or software. The firmware and/or software may configure the connector ports to control and/or designate various electrodes as anodes, cathodes, and/or anode-cathode pairs.

The power source 54 provides power to one or more components of the implantable system 10. The power source 54 may include an energy-storage device, such as a battery. In some examples, the power source 54 may be a rechargeable battery or a non-rechargeable battery. For example, the power source 54 (e.g., a rechargeable battery) may be configured to be wirelessly recharged by a wireless charger outside of the patient's body. A non-rechargeable battery requires replacement through surgical procedures.

Several factors influence battery design and efficiency. For example, respiratory training sessions during the day typically use lower stimulation energies compared to sleep therapy. For example, respiration conditioning stimulation may involve intensities such as 2 mA compared to higher sleep mode requirements, significantly reducing battery drain. This energy efficiency ensures the rechargeable battery can support frequent respiration conditioning training sessions without depleting rapidly.

Effective respiration conditioning training may reduce the amount of stimulation needed at night, preserving battery life further. For instance, training may enhance patient compliance with synchronizing breath to the stimulation and/or acclimation to stimulation sensations. As a result, the sleep disordered breathing therapy may require less gradual acclimation as the patient may better tolerate a faster ramp up to higher stimulation currents with more efficacy in regard to sleep apnea neurostimulation treatment. Additionally, or alternatively, the patient may be less susceptible to awakening at night due to physical and/or psychological discomfort with sleep apnea neurostimulation treatment and/or may be less inclined to resist breathing in synchrony with stimulation. As another possible advantage, the patient may entrain more readily during neurostimulation for sleep disordered breathing. These effects may additionally decrease overall energy consumption of the IPG as the patient may be able to progress faster to the most effective sleep apnea stimulation profiles.

Awake respiratory conditioning may decrease the intrinsic breathing rate of the patient and thus allow a lower stimulation frequency during sleep therapy. In turn, this may reduce the overall energy demand during sleep therapy and thereby extend battery life without compromising therapy efficacy.

The rechargeable battery is designed to be recharged easily by the patient, with procedures integrated seamlessly into their daily routine. The system may use a magnetic inductive charging wand, which is already part of the device's setup for communication and telemetry. This wand could be enhanced to serve a dual purpose: enabling both recharging and starting/stopping therapy. Patients could place the wand over the implanted device for a specified period (e.g., 30-60 minutes) to recharge the battery. The interface on the wand or companion app could indicate the charging status and battery level.

In some embodiments, a single device may combine recharging and therapy control to simplify the process for patients. For instance, the wand or a smartphone application may include controls to start or stop therapy while recharging the battery. This integration ensures patients only need one device to manage their therapy.

Some embodiments may include a two-battery hybrid system. The hybrid battery design may include a rechargeable battery powering respiration conditioning respiration training and a replaceable battery for sleep therapy. This approach minimizes the risk of battery depletion during the sleep mode period, ensuring consistent therapy delivery while allowing the rechargeable battery to focus on respiration conditioning usage. Some embodiments may include a fully rechargeable battery.

Patients may recharge the device daily, typically during non-therapy periods, such as in the afternoon. This design prioritizes convenience while ensuring that the battery can support both respiration conditioning and sleep therapy effectively.

By improving the efficiency of sleep therapy, respiration conditioning training reduces the battery's overall burden. For example, stronger diaphragm muscles resulting from respiration conditioning may require less stimulation intensity during sleep therapy, prolonging battery life.

In various examples, the processor 50 is configured to process information and control operation of the system 10, such as by controlling the delivery of electrical stimulation, for example, as one or more stimulation bursts, to a target nerve. Controlling the delivery of the electrical stimulation may include controlling operation of the principal stimulation circuitry 42 and/or the subordinate stimulation circuitry. The processor 50 may include one or more processors to execute instructions stored on one or more non-transitory computer-readable media, such as the memory 56. Accordingly, when the system 10 is described as determining information or parameters, executing operations such as comparing values to thresholds, modifying parameters for electrical stimulation, sensing or obtaining parameters or information, and so forth, the processor 50 may be performing at least some of these operations.

The memory 56 may include one or more of memory devices, one or more storage devices, or a combination thereof. For example, the memory 56 may include one or more non-transitory computer-readable media. The one or more non-transitory computer-readable media may store instructions that, when executed by the processor 50, cause the processor 50 to execute operations discussed herein, such as titrating a stimulation energy. The memory 56 may also store other information, such as sensed data, user preferences, control commands from a physician, and so forth.

In operation, electrical stimulation burst(s) generated by the IPG 12 travels along the leads 14 and/or 16. The leads 14 and/or 16 include electrodes to deliver the electrical stimulation to a target nerve. For example, the phrenic nerve stimulation leads 14 include phrenic nerve stimulation electrodes 34 and the airway nerve stimulation leads 16 include upper-airway nerve stimulation electrodes. Stimulation burst(s) provided for a period of time to an electrode results in electrical stimulation of the nerve or other target tissue. The energy of the stimulation and the duration of the period of time, potentially amongst other factors, determine the stimulation power provided to the target tissue. The electrical stimulation activates the fibers (that is, generates an action potential) causing a functional response by the nerve. A higher energy stimulation may activate more nerve fibers resulting in a larger functional response. A lower energy stimulation may activate fewer nerve fibers resulting in a smaller functional response.

For a particular functional response, such as the inducement of the contraction of a muscle (for example, the diaphragm), a minimum threshold energy may be required. The strength of the functional response (for example, the strength and/or extent of a muscular contraction needed to achieve a desired effect on respiration) determines the minimum threshold energy. In order to provide a range of functional responses (for example, a range of strengths and/or extents of muscular contractions to create a range of effects on respiration), a device, such as the IPG 12, may be configured to provide electrical stimulation to satisfy various energy thresholds. For example, there may be weak, medium, and strong thresholds to describe a range of functional responses.

The IPG 12 may receive sensed signals from one or more sensors, such as the external sensors 18, the IPG sensors 19, and/or the distributed sensors 22. Each of the sensors 18, 19, and/or 22 may include any type and/or number of sensors. The sensors 18, 19, and/or 22 may include one or more sensors such as one or more of motion sensors (for example, accelerometers), pressure sensors, acoustic sensors, transthoracic-impedance sensors, light sensors, airflow sensors, microphones, ultrasonic transducers, heart rate sensors, blood oxygenation sensors (for example, pulse oximeters), muscular electrical activity sensors (for example, electromyography sensors), peripheral arterial tone sensors, or other types of sensors. In some examples, one or more of the leads 14 and/or 16 may include and/or act as sensors. For example, the phrenic nerve electrodes 34 and or the airway nerve stimulation leads may include or provide for one or more sensors 32a and 32b, respectively. For example, a measurement of impedance between the pulse generator 12 and the phrenic nerve electrodes 34 may function as a transthoracic impedance sensor. Implanted and/or external accelerometers, such as the accelerometer 36, may be single axis accelerometers, or may be two- or three-axis accelerometers.

The external sensors 18 are external to the patient (that is, not implanted). External sensors 18 may communicate wirelessly with the IPG 12 via a wireless communications link 68. The external sensors 18 may be wearable or otherwise coupled to the patient. The external sensors 18 may be communicatively coupled to one or more of the implantable device 12 or the external computing device 210 and may be electrically coupled to the external computing device 210. The external sensors 18 may provide sensed information to the IPG 12 via a wireless communication connection 68. The wireless communication connection 68 may enable wireless communications to be exchanged between the external sensors 18 and the implantable system 10 in a similar manner as the wireless communication connection 30. For example, wireless communications may be exchanged pursuant to a short-range wireless communication protocol such as, for example, the Bluetooth® wireless communication protocol, or inductive telemetry, or another telemetry method.

In at least one example, any one or more of the sensors 18, 19, and/or 22 may include transthoracic-impedance sensors configured to measure an impedance across a patient's thorax. Furthermore, in some examples, the phrenic nerve electrodes 34 include the TTI sensor(s) 32. The transthoracic-impedance sensor data may be indicative of, for example, chest size, chest motion (that is, changes in chest size), cardiac parameters, and/or respiratory parameters that may be indicative of a degree of arousal and/or patient motion. For example, patient motion may include a patient activity like coughing or snoring that correlate with a change in degree of arousal. As another example, the transthoracic impedance data may be indicative of a change in lung volume due to changes in the respiratory pattern of a patient caused by a change in patient position. Thus, while the transthoracic impedance may not directly measure patient motion in the manner of an accelerometer, the transthoracic impedance may serve as a proxy or predictive measurement of patient motion.

Transthoracic-impedance data may be representative of, and/or may be used to determine, a patient's respiratory rate (for example, based on a frequency of the signal from the transthoracic-impedance sensor) and/or a patient's tidal volume (for example, based on an amplitude of the signal from the transthoracic-impedance sensor). The external sensors 18 may additionally or alternatively include other sensors, such as one or more of a heart-rate sensor, an optical sensor, a pressure sensor, an effort belt, an electrocardiogram sensor, an electroencephalogram sensor, and/or other types of sensors.

The IPG sensors 19 may include one or more sensors on or within the IPG 12. For example, the at least one accelerometer 36 is an implantable accelerometer as opposed to the external sensors 18. For example, the at least one accelerometer 36 may be housed within the IPG 12. The at least one accelerometer 36 may be configured to sense a position and/or movement of a patient. The IPG sensors 19 may be implantable within a patient, at least because the IPG 12 may be implantable within a patient. Because the at least one accelerometer 36 may be implantable within a patient, the processor 50 may use the position and/or movement information sensed by the at least one accelerometer 36 to determine, for example, a position of a sleeping patient to determine when a patient rolls from one position to another and/or a pitch of a patient to determine when a patient sits up because they are awake.

The distributed sensors 22 may include one or more sensors electrically coupled to the IPG 12, but not physically located within a housing of the IPG 12. For example, the distributed sensors 22 may include one or more pressure sensors implantable within a patient. Pressure sensors implanted within a patient may measure changes in pressure caused by a patient breathing as the patient's lungs expand and contract, or may measure changes in pressure caused by a patient's heart beating as the patient's heart expands and contracts, or may measure other changes in pressure. The distributed sensors 22 may include at least one accelerometer in addition to, or in lieu of, other sensors, such as the pressure sensors. In an example, an accelerometer on a lead proximate to the hypoglossal nerve, or located elsewhere proximate to the airway, may detect motion indicative of airway patency. For example, at least one accelerometer may be implemented on one or more of the optional additional leads 13. The distributed sensors 22 may be electrically coupled to the IPG 12 to provide sensed information to the IPG 12.

The external computing device 210 may include, for example, a computing device operated by a physician. A physician may use the external computing device 210 to control and/or program the implantable system 10. in some examples, the external computing device 210 communicates with IPG 12 of system 10 through a communication connection 30. In some examples, the communication connection 30 can be a wireless communication connection. For example, the wireless communication link 30 can be a telemetry communication connection and/or a Bluetooth® wireless communication connection between suitable circuitry in each of computing device 215 and the implanted (or implantable) pulse generator (IPG) 12 of system 10. For example, the physician may use the external computing device 210 to control and/or program the entrainment engine 24 to modify parameters of stimulation delivered by the IPG 12. Those parameters may include parameters discussed in regard to FIG. 10, for example, one or more of a minimum stimulation energy, a maximum stimulation energy, a number of stimulation bursts, a frequency of stimulation bursts, a width of the stimulation bursts, a stimulation burst amplitude, a maximum stimulation burst amplitude, a minimum stimulation burst amplitude, a stimulation current, a stimulation voltage, a stimulation polarity (for example, monophasic or biphasic), stimulation energy ramps, and so forth. As discussed in regard to FIG. 10, the stimulation bursts may be provided as pulse trains and therefore the parameters may include pulse train parameters.

Stimulation of a phrenic nerve (for example, stimulation of the thoracic phrenic nerve and/or the cervical phrenic nerve by the IPG 12) to produce rhythmic contractions of one or both hemidiaphragms innervated by the stimulated nerve and consequent rhythmic lung inflations may synchronize the patient's respiratory drive with the stimulation. When the patient's respiratory drive is synchronized in this manner during phrenic nerve stimulation treatment for sleep disordered breathing, the patient may be referred to as entrained.

Indicators of synchronization may include a respiratory rate of the patient that is clustered at or near the programmed rate of the stimulation. In addition, the contralateral hemidiaphragm may move in synchronization with the stimulated hemidiaphragm. Unilateral stimulation of a hemidiaphragm may provide some improvements in oxygenation and other respiration and/or sleep metrics even without synchronization. However, the unilateral stimulation with synchronization provides more substantial improvements in respiratory function. Thus, synchronization may be a beneficial and important goal for disordered breathing therapy. With entrainment, spontaneous breathing and central respiratory drive are preferably preserved. Thus, an ordered rhythmic breathing pattern is established without a patient becoming dependent on the phrenic nerve stimulation. The patient is thus said to be “entrained” as opposed to “paced” by the nerve stimulation.

In one example, IPG 12 (e.g., the entrainment engine 24 of the processor 50) may receive and process a signal representative of patient respiration to generate a histogram frequency distribution of patient breath lengths over a signal collection period, identify a stimulation frequency band (SFB) of sensed lengths in the histogram that is centered on a stimulation frequency, calculate a first sum of occupancies of breath lengths in a range of the stimulation frequency+/−a selected band width, calculate a second sum of occupancies of all of the lengths of sensed breaths (a respiratory frequency band [RFB]), and calculate the capture index as a ratio of the first sum over the second sum. In another example, the IPG 12 (e.g., the entrainment engine 24 of the processor 50) may determine the capture index based on a ratio of a variance of the SFB to a ratio of the total variance across the histogram. In yet a further example, the IPG 12 (e.g., the entrainment engine 24 of the processor 50) may determine the capture index by dividing the spectral power in an SFB by the spectral power in an RFB.

The SFB includes a range of frequencies at which electrical stimulation (or stimulation pulse train) is applied to the patient 20 by the system 10. The RFB includes a range of frequencies at which the patient 20, or a human being in general, naturally respirates while asleep (for example, 0.5-1 Hz, 0.1 to 0.5 Hz, or another range). The capture index may represent the fraction of total spectral plot of respiration that falls into the SFB, and/or may implement a time-domain-based comparison of the relationship between the electrical stimulation and the breathing of the patient 20. For example, the time-domain-based comparison may include detecting a time between inhalation peaks over a certain period of time and determining, over that time, the number of breaths that are within a threshold of the programmed rate of treatment. In some examples, the value of capture index may increase proportionally with the entrainment of respiration by stimulation. The stimulation frequency thus becomes the dominant frequency of the respiration signal as entrainment increases.

The IPG 12 may control the electrical energy delivered to the leads 13 to treat decreased respiratory drive (that is, to cause the diaphragm to contract due to stimulation of the phrenic nerve) and/or to improve or restore airway patency (that is, to cause movement of muscles in the upper-airway due to stimulation of an upper-airway nerve such as, for example the hypoglossal nerve). For example, the IPG 12 may deliver electrical stimulation to one or more target nerves via the phrenic nerve stimulation lead(s) 14 and/or the airway nerve stimulation lead(s) 16. Such target nerves may include, for example, one or more of a phrenic nerve, a hypoglossal nerve, the ansa cervicalis, other nerves that activate a diaphragm and/or modify a position of airway tissue which may include the tongue, and so forth.

In various examples, the IPG 12 may deliver electrical stimulation signals to one or more of the leads 13. The leads 13 may each include electrodes to deliver electrical stimulation to a target nerve based on the electrical stimulation signals. For example, the processor 50 may cause the stimulation circuitry 42, 44 to deliver the electrical stimulation to the leads 13. The processor 50 causing the stimulation circuitry 42, 44 to deliver stimulation may include controlling one or more switching elements, such as one or more metal-oxide-semiconductor field-effect transistors (MOSFETs), bipolar junction transistors (BJTs), relays, diodes, other switching elements, a combination thereof, and so forth, within the stimulation circuitry 42, 44 to modulate power (for example, derived from the power supply 54) and produce an electrical stimulation with desired electrical stimulation parameters.

In at least one example, the processor 50 may include and/or execute the operational mode controller 156 to control the stimulation parameters of the electrical stimulation provided by the stimulation circuitry 42, 44 according to a stimulation profile (e.g., as selected from the one or more stimulation profiles 58). The stimulation profile may include electrical stimulation parameters dictating electrical parameters of the stimulation bursts. The operational mode controller 156 may dictate parameters of a stimulation provided by the principal stimulation circuitry 42 and of a stimulation provided by the subordinate stimulation circuitry 44 according to the stimulation profile selected by the operational mode controller 156 from the one or more stimulation profiles 58 of the stimulation profile library 57A. A stimulation profile of stimulation provided by the principal stimulation circuitry 42 may be different than a stimulation profile of stimulation provided by the subordinate stimulation circuitry 44. The electrical stimulation parameters may be upper-airway nerve electrical stimulation parameters (e.g., hypoglossal nerve electrical stimulation parameters and/or ansa cervicalis electrical stimulation parameters) and/or phrenic nerve electrical stimulation parameters.

Stimulation profiles (e.g., as stored in the stimulation profile library 57) may thus include one or more electrical stimulation parameters. Stimulation profiles being different than one another may include at least one electrical stimulation parameter of one stimulation profile being different than at least one electrical stimulation parameter of another stimulation profile. Similarly, stimulation profiles being the same may include the stimulation parameters of one stimulation profile being the same as the stimulation parameters of another stimulation profile.

As discussed elsewhere herein, the processor 50 may control timing of delivery of the principal stimulation by the principal stimulation circuitry 42 and of subordinate stimulation delivered by the subordinate stimulation circuitry 44. The processor 50 may include and/or execute the operational mode controller 156 and the channel coordination timer 27 to coordinate the relative timing of the subordinate and principal stimulations. As discussed in greater detail elsewhere herein, one or more aspects of timing of the subordinate stimulation may be dictated by one or more aspects of timing of the principal stimulation. Thus, the timing of the subordinate stimulation may be controlled relative to the timing of the principal stimulation.

In some examples, the IPG 12 may be programmed to initiate disordered breathing therapy with a delivery of stimulation energy to one or more target nerves based on satisfaction of one or more sleep indication conditions. The processor 50 may include and/or execute the operational mode controller to determine whether one or more sleep indication conditions are met to evaluate whether the patient is asleep.

The sleep indication conditions may include detected movement of the patient. For example, movement detected by one or more accelerometers (including, for example, the accelerometer 36) may provide movement data that may serve as a proxy for the actual sleep state of the patient. One or more of patient activity, motion, position, and pitch as detected, for example, by an accelerometer, may be indicative of sleep particularly when combined with a time of day that corresponds to a regular sleeping time of the patient. Thus, the sleep indication conditions may further include pre-programmed stop and start times, a pre-programmed duration, and pre-programmed thresholds for patient pitch and patient movement. Optionally, the sleep indication conditions may include a patient position (for example, a patient position in one of four positional quadrants including a left-side position, a right-side position, a prone position, and a supine position) and/or changes in physiological parameters, such as a reduction in heart rate. The IPG 12 may be pre-programmed to deliver therapy according to a regular schedule and may use the patient motion or position data to titrate that pre-programmed delivery.

In some examples, the IPG 12 may modulate the intensity of the stimulation based on feedback information indicative of the efficacy of the treatment in addition to, or in lieu of, modulating the stimulation energy based on pre-programmed parameters. Feedback information may include, for example, an entrainment index. As discussed above, an entrainment index may be determined based on a patient's breathing rate.

The IPG 12 may receive information indicative of the patient's breathing rate from one or more respiration sensors and determine the entrainment index based on the patient's breathing rate. Respiration sensors may include sensors configured to provide information indicative of a patient's respiration. For example, transthoracic impedance sensors such as the transthoracic impedance sensor (TTI) 32 may be respiration sensors, at least because the transthoracic impedance information may be used to determine a patient's breathing rate and/or tidal volume. Alternatively or additionally, the respiration sensor may be an accelerometer.

The processor 50 may include and/or execute the respiration detector to detect patient respiration (including, for example, the patient's breathing rate) based on sensed information. For example, the TTI sensors 32 may send sensed information to the processor 50 which may be used to determine the patient's breathing rate. In some examples, the distributed sensors 22 and/or the external sensors 18 may include at least one transthoracic-impedance sensor. For example, the distributed sensors 22 may include a lead that forms a cathode-anode pair with the housing of the IPG 12 itself. That is, the lead may be used to measure an impedance between the lead and the IPG 12, and this impedance may be a transthoracic impedance which may be used to determine breathing rate. The respiration detector 25 may use the transthoracic-impedance data to detect patient respiration parameters, such as breathing rate and/or tidal volume. The respiration detector 25 may additionally or alternatively use sensed information to determine an entrainment index in some examples, and/or may use the sensed information to classify patient respiration, such as by identifying certain patterns of respiration as being or including Cheynes-Stokes respiration (CSR).

In examples in which the IPG 12 receives feedback information indicative of patient respiration from the respiration detector 25 and/or patient-sleep-condition information from the operational mode controller, the IPG 12 may modulate the energy of the stimulation based on the feedback information. For example, the operational mode controller 156 may modulate one or more stimulation parameters based on the feedback information, and the channel coordination timer 27 may modulate timing of electrical stimulation based on the feedback information. Accordingly, the operational mode controller 156, operational mode controller, respiration detector 25, and channel coordination timer 27 may all operate in combination with one another in some examples to deliver and modulate electrical stimulation. Such modulation may be subject to an upper bound of stimulation energy as determined by a pre-programmed limit or as determined by an indication from the feedback information that the patient's respiration has reached a desired condition. Such a desired condition may include, for example, that the patient is successfully entrained.

FIG. 11C schematically shows details of the implantable components 10 of the respiration conditioning system 5 of FIG. 1. As illustrated in FIG. 11C, the implantable components 10 include leads 11A, which include the phrenic nerve stimulation leads 14 and/or the upper-airway stimulation leads 16, and the IPG 12. The phrenic nerve leads 14 include a plurality of phrenic nerve electrodes 306, and the airway nerve leads 16 include a plurality of upper-airway nerve electrodes 308. The electrodes 306 and/or 308 may be transvenous lead electrodes and/or nerve cuff electrodes. The electrodes 306 and/or 308 may serve as distributed sensors and/or the stimulation leads may include distributed sensors. In some examples, the implantable components may include additional leads 399 without stimulation electrodes but which include the distributed sensors.

The one or more phrenic nerve leads 14 may be configured for implantation in a blood vessel proximate to the phrenic nerve or may include a nerve cuff configured for implantation around the phrenic nerve. The one or more upper-airway stimulation leads 16 may be configured for implantation in a blood vessel proximate to the hypoglossal nerve or may include a nerve cuff configured for implantation around the hypoglossal nerve. For example, the leads 14, 16 may be sized and/or shaped in a configuration that allows the leads 14, 16 to be implanted in, and remain stable in, a respective target blood vessel without damaging the respective target blood vessel. Alternatively or additionally, the leads 16 may include leads configured for implantation in a blood vessel proximate the ansa cervicalis or other upper-airway nerve or may include a nerve cuff configured for implantation around the ansa cervicalis or other upper-airway nerve. Where a nerve has a left and right occurrence, the leads 11 may include leads configured to service one or both of the occurrences (for example, the left phrenic nerve, the right phrenic nerve, or both).

The IPG 12 may include, and/or be coupled to, one or more sensors 19 as shown in FIG. 11A. In some examples, leads 14 and/or 16 may include one or more sensors 32a and 32b, respectively as shown in FIG. 11A. In other examples, the system 10 may include sensor 22 that may be distributed, for example on additional non-stimulation leads 26, as shown in FIG. 11A, or as implanted at other locations on the patient. In various examples, the sensors may be configured to sense information indicative of treatment provided by the system 10, respiration of the patient 20, information related to the respiration of the patient 20, and so forth. Examples of particular types of sensors are provided below. In some examples, the lead 14 may be a dedicated stimulation lead and the lead 16 may be a dedicated sensing lead. In such examples, the lead 14 may include stimulation electrodes but exclude sensing electrodes and/or other sensors, and the lead 16 may include sensing electrodes and/or other sensors and exclude stimulation electrodes.

In various examples, the one or more leads 11 may be an intravascular stimulation lead placed proximate a target nerve to send signals from the IPG 12 to muscles that control breathing (for example, the diaphragm) and/or airway patency. The one or more leads 14 if implanted in the thoracic cavity may provide a measurement of transthoracic impedance indicative of physiological signals such as breathing, fluid state, pulse rate, and so forth. Examples herein may refer to the one or more leads 11 in singular form (the lead 14 or the lead 16) for simplicity but such examples are not limited to a single lead and may include one or more leads 11. Similar principles may be applicable to the one or more leads 16 (which may be referred to as the lead 16 for simplicity), but may not be repeated for purposes of brevity.

Electrodes may include any of various electrodes, such as ring electrodes (that is, electrodes spanning around a circumference of the lead) or other types of electrodes. In some examples, a lead may include a single electrode. In other examples, a lead may include multiple electrodes. A position of the electrode(s) may be selected to correspond to a position of a target tissue relative to the lead when the lead is implanted such that an electrical stimulation applied to the electrode is provided to the target tissue. In some examples, multiple electrodes may be positioned close together in a location corresponding to a position of the target tissue. For example, when viewing a radial cross-section of a spiral or coiled portion of a lead, it may be advantageous for all of the electrodes of the lead to be located within a single quadrant of the radial cross-section.

In some examples, the lead 11 may include or be coupled to stimulation-delivery elements in addition to, or alternative to, electrodes. For example, the lead 11 may include or be coupled to one or more nerve cuffs. Once implanted, the nerve cuff may be disposed circumferentially around a target nerve.

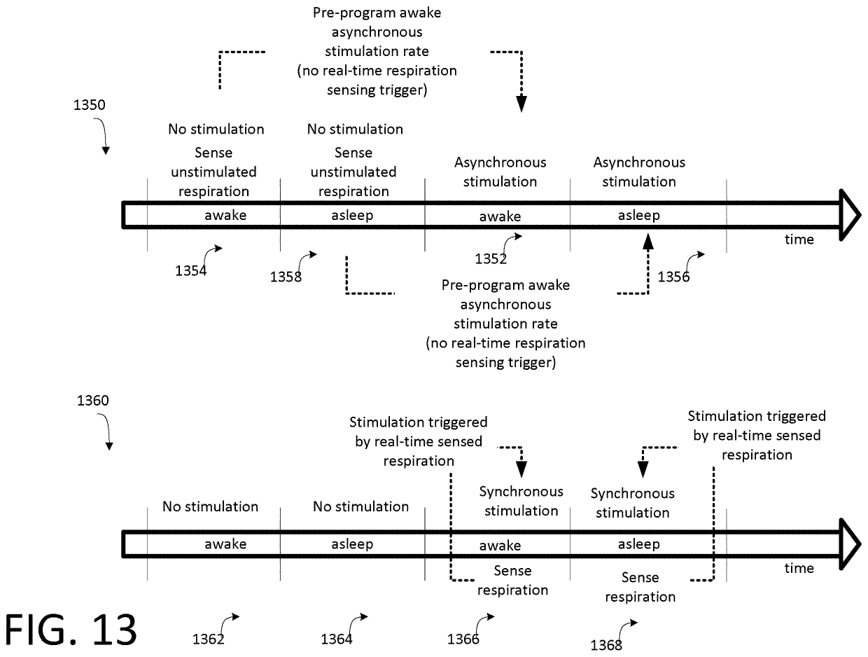

In various examples, the implanted system 10 may provide sleep mode or non-sleep mode stimulation therapy in an asynchronous sub-mode or a synchronous sub-mode. These modes are illustrated schematically in FIG. 13 as discussed below. The asynchronous sub-mode is a mode of therapy delivery in which the stimulation rate is pre-determined and stimulation is not triggered by respiration sensed in real-time. In an implementation, the IPG 12 may determine the stimulation rate based on an intrinsic breathing rate for the patient as measured in an absence of stimulation and determined prior to delivery of stimulation. The synchronous sub-mode is a respiration-triggered mode in which the stimulations are triggered by respiration sensed in real-time. In an implementation, the synchronous sub-mode may include a back-up apnea triggered asynchronous stimulation delivery. In such a stimulation delivery, the stimulation rate may be determined prior to delivery of stimulation similarly to the asynchronous sub-mode. However, in the back-up delivery, the stimulation may be provided only in response to a lack of detected breath, in other words, detection of an apnea. In various examples, a patient may be experiencing an apnea when the patient's respiration remains below a threshold value for at least a threshold period of time. For example, a patient may be experiencing an apnea if the amplitude of the patient's respiration does not exceed a threshold value within, say, 3-10 seconds. Modification of the electrical stimulation therapy by the IPG 12 as discussed herein may depend on the therapy mode. For example, the IPG 12 may modify the therapy based on the therapy-specific indicator of capture index (described in more detail elsewhere herein) in the asynchronous sub-mode but not in the synchronous sub-mode. In this mode, the IPG 12 may modify electrical stimulation current to modify therapy. As another example, the IPG 12 may modify pulse duration for the electrical stimulation signal to modify therapy in the synchronous sub-mode.

The IPG 12 may provide the electrical stimulation as a pulse train, for example, as described with regard to FIG. 10. Modification of the electrical stimulation therapy may include modification of one or more parameters to increase or decrease the stimulation energy provided by the electrical stimulation as discussed in regard to FIG. 10.

It should be reiterated that the representations of FIGS. 11A, 11B, and 11C are significantly simplified representations of an actual system 5. Those skilled in the art should understand that such a device has other physical and/or functional components, such as central processing units, other packet processing modules, and short-term memory. Accordingly, this discussion is not intended to suggest that FIGS. 11A, 11B, and 11C represent all of the elements of the system 5.

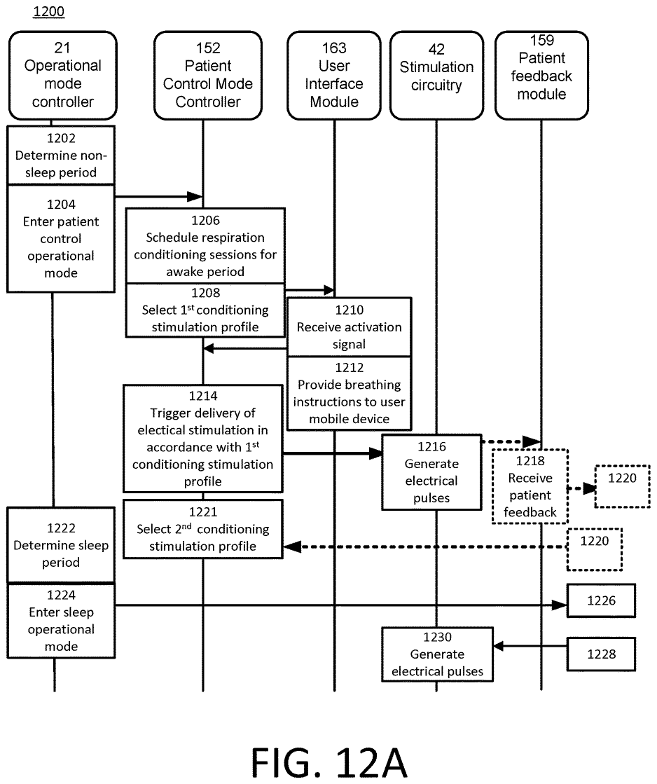

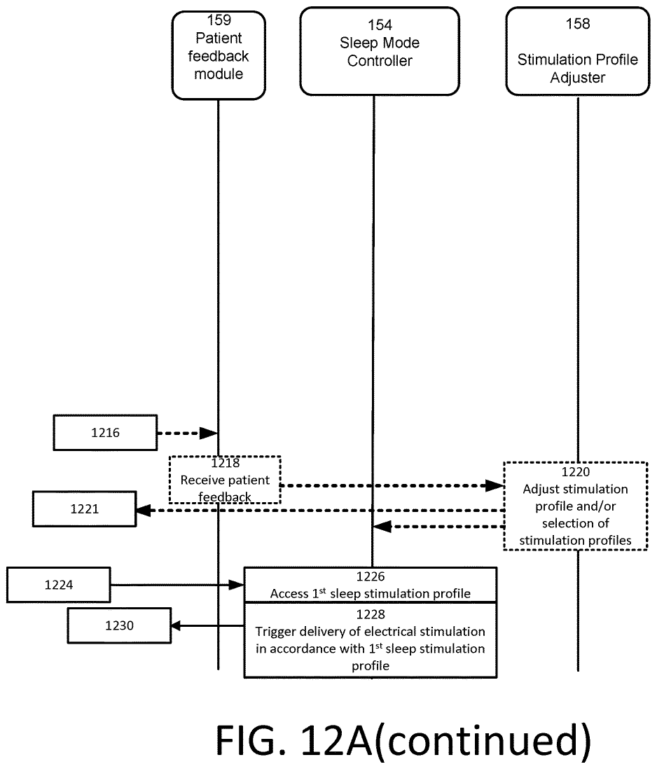

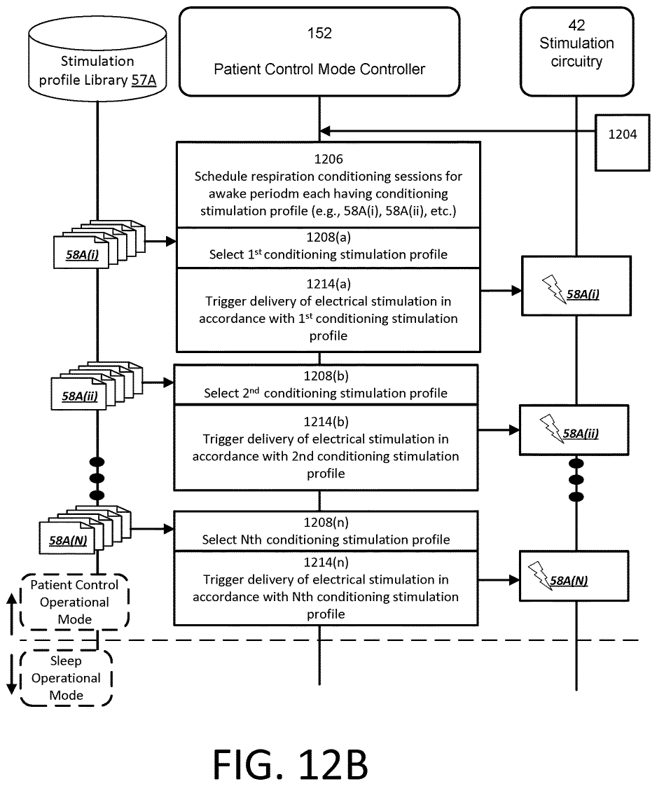

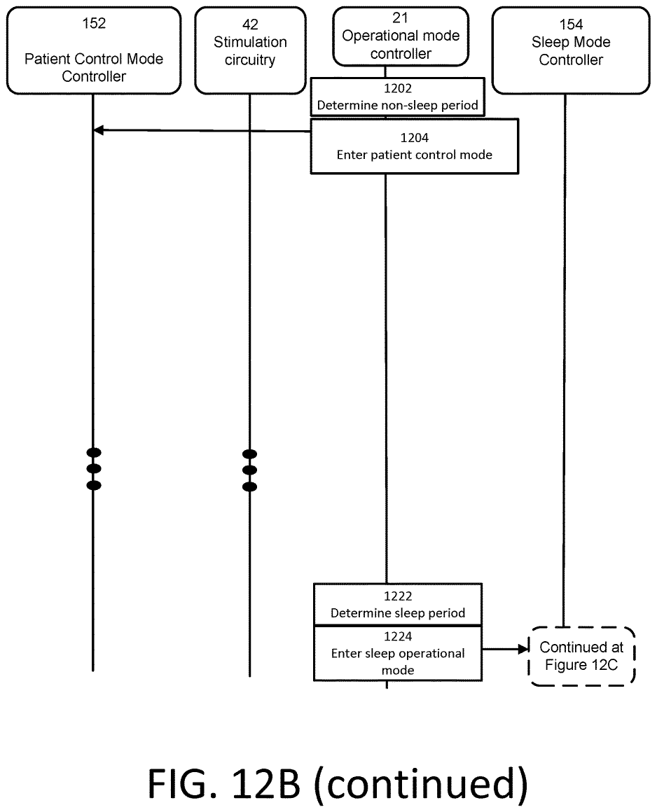

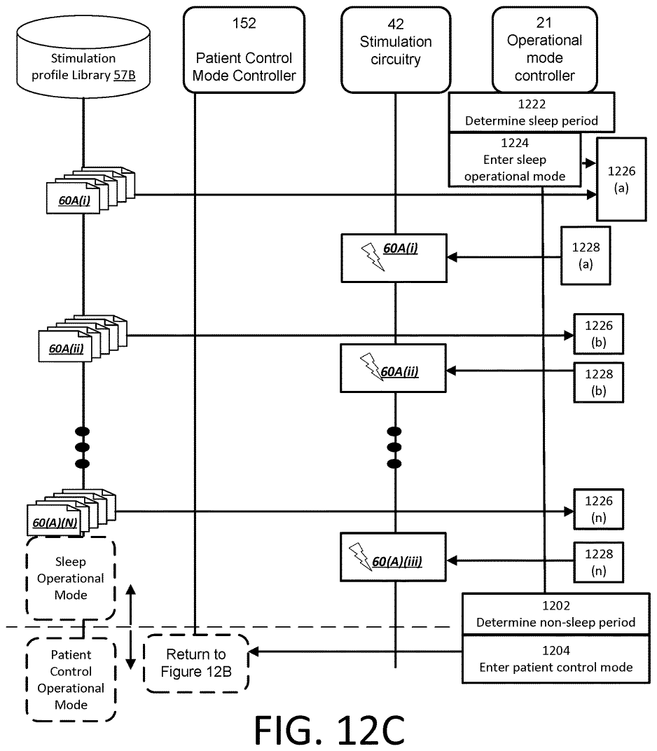

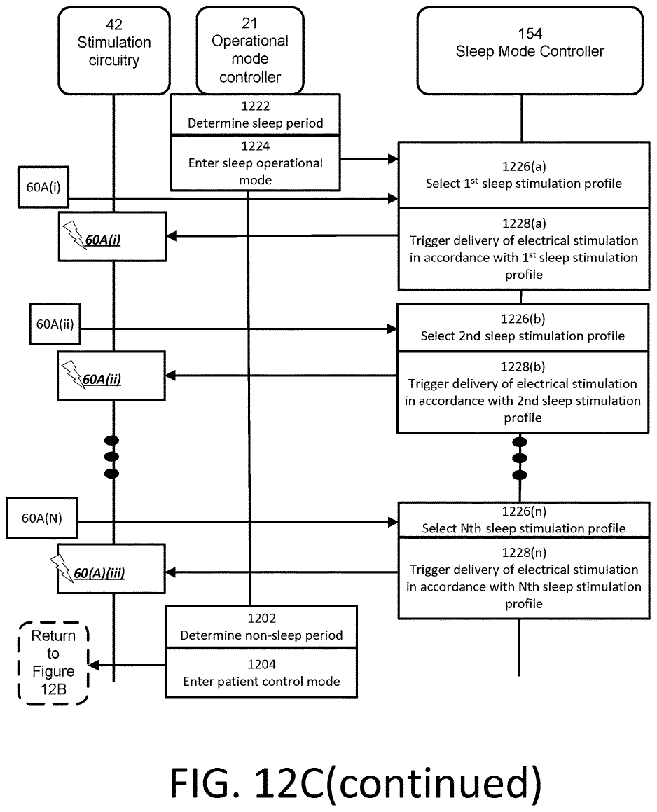

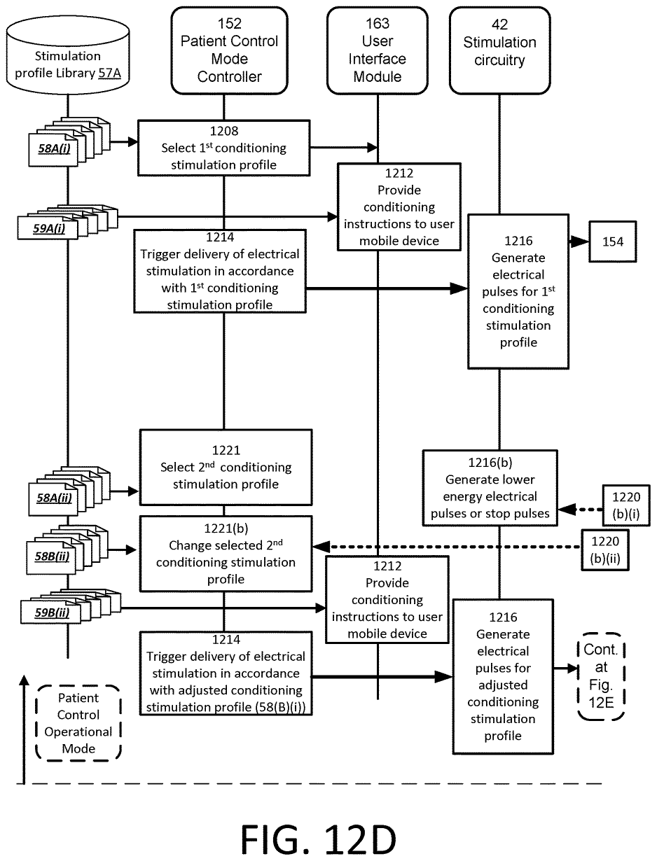

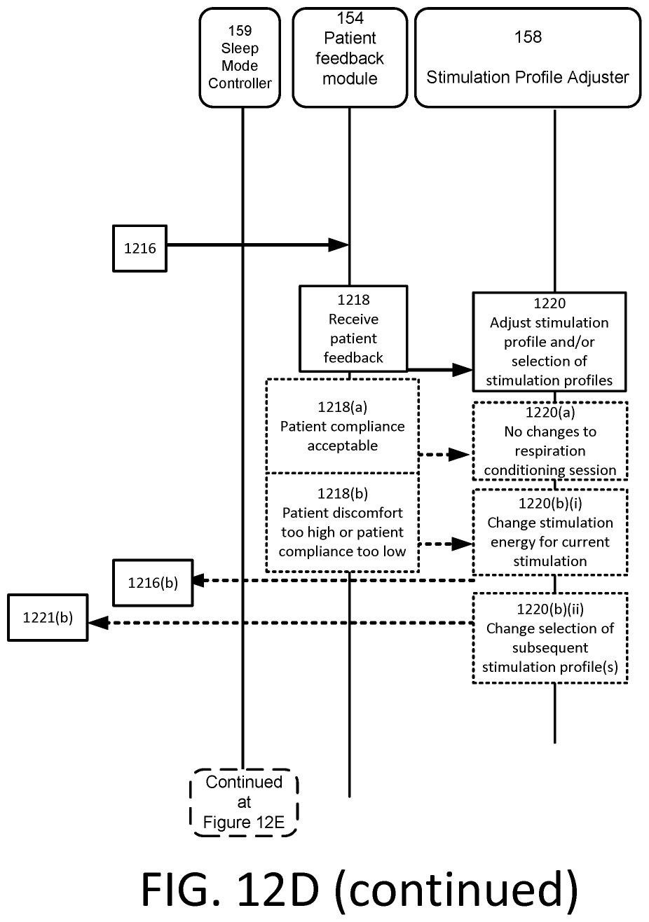

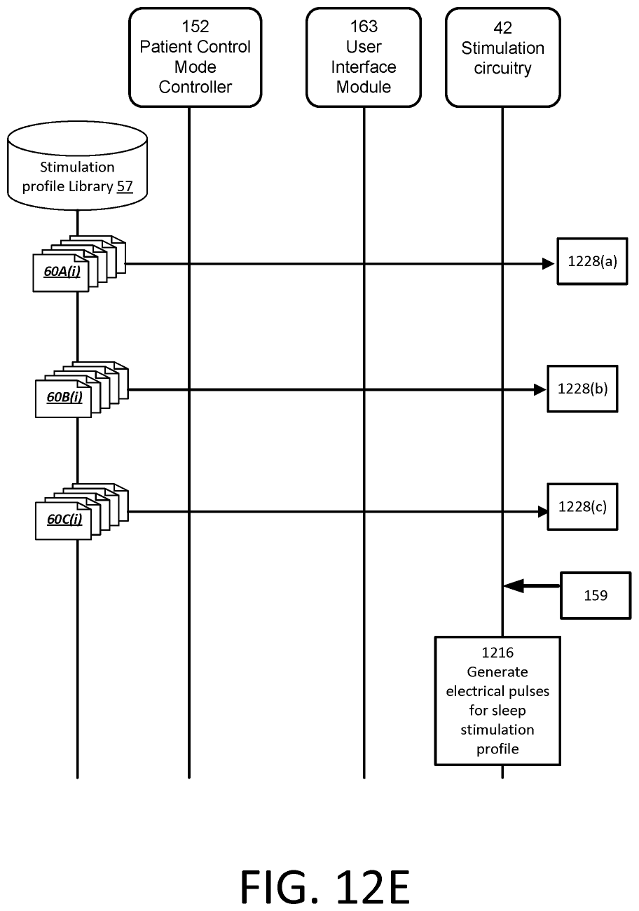

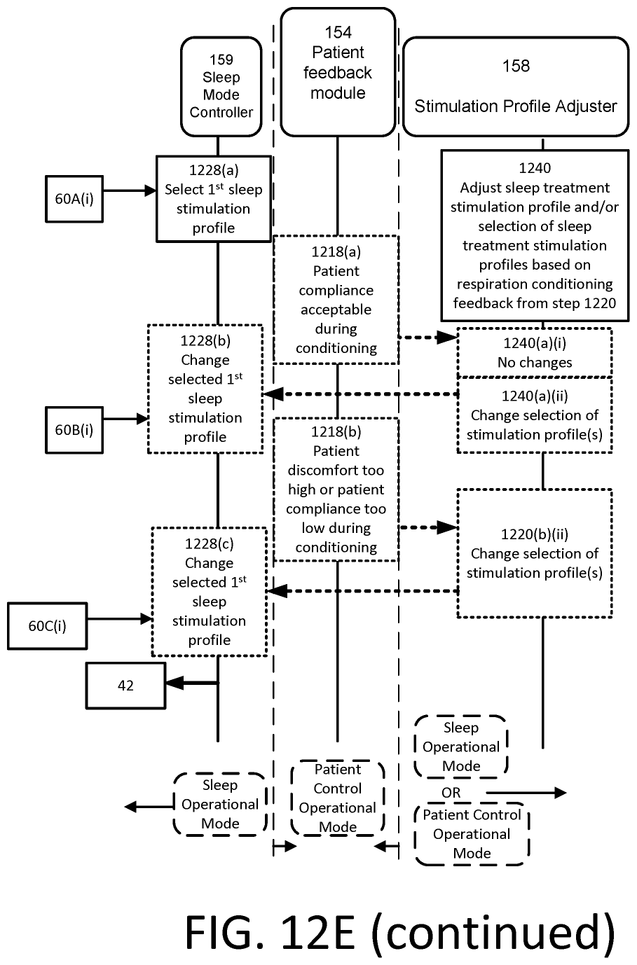

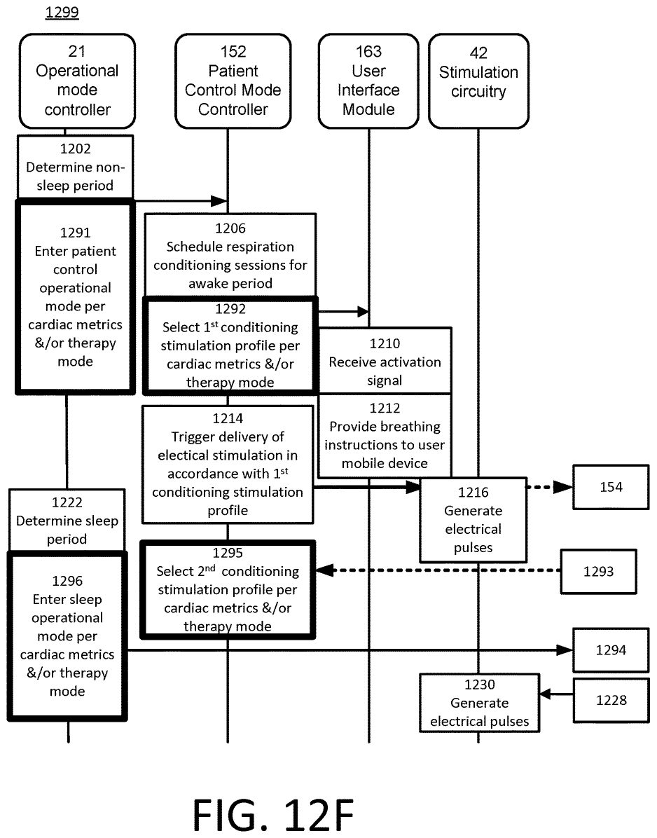

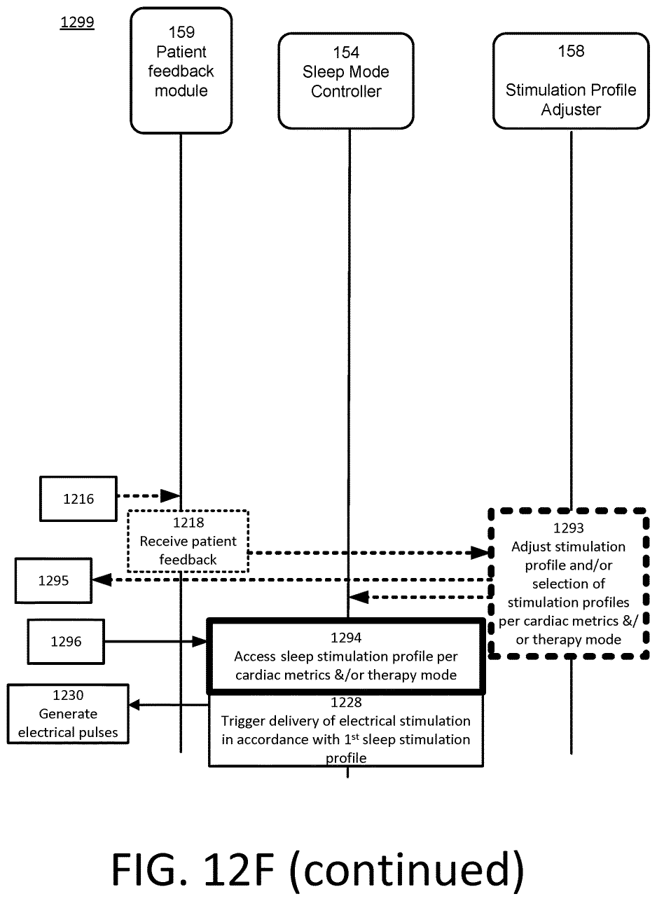

FIGS. 12A-12E illustrate swim-lane diagrams 1200, depicting operation of the processor 50. FIG. 12F illustrates swim-lane diagram 1299, depicting operation of the processor 50. The operations shown in FIGS. 12A-12F may include additional steps and some of the steps may be performed in a different order than that shown, or at the same time. Furthermore, some of these steps may be optional in some embodiments. Accordingly, the FIGS. 12A-12F are merely exemplary of one process in accordance with illustrative embodiments described herein. Those skilled in the art therefore can modify the process as appropriate.

The system 10 provides a method for adaptive respiratory stimulation through dual operational modes: patient control mode for non-sleep periods and sleep operational mode for sleep periods. The system includes an operational mode controller, a patient control mode controller 152, a user interface module 163, stimulation circuitry, a patient feedback module 159, a sleep mode controller, and a stimulation profile adjuster 158. These components work together to deliver personalized respiratory therapy tailored to the patient's needs in both awake and sleep scenarios.

Referring to FIG. 12A, at step 1202, the operational mode controller 156 determines when there is a non-sleep period and transitions the system into patient control mode (Step 1204). Although shown as beginning with 1202 and 1204 to determine a non-sleep period and provide patient controlled conditioning and then proceeding to 1222 and 1224, the IPG 12 may begin with 1222 and 1224, deliver sleep therapy, and then proceed to 1202 at the completion of a sleep therapy. In some examples, the IPG 12 may bypass delivery of sleep therapy or non-sleep therapy as described herein. As described previously, the non-sleep period may be pre-determined and entered in by the patient and/or the medical practitioner. In an embodiment the determination of the non-sleep period could be a result of the patient putting the device in the patient control mode (e.g., via the user interface 220). Similarly, the patient could put the device in the sleep mode. Thus, in various embodiments, steps 1202/1204 could be one step or two; similarly, steps 1222 and 1224 could be one step or two.

In this mode, the patient control mode controller 152 schedules respiratory conditioning sessions (Step 1206). The patient control mode controller 152 enables the scheduling of respiratory conditioning sessions that can be tailored to suit the patient's lifestyle and therapeutic needs. These sessions may range from all-day training periods to shorter, focused training sessions lasting 10-60 minutes, scheduled 1-4 times per day. The flexibility in scheduling allows the patient to integrate therapy seamlessly into their daily routine while ensuring effective respiratory conditioning.

Sessions may be pre-programmed by the clinician or system during setup to establish a structured training regimen. For example, a patient with a high need for respiratory conditioning might follow a pre-programmed schedule of 30-minute sessions, four times a day, spaced evenly throughout waking hours. This approach ensures consistent stimulation and gradual improvement in patient acclimation to therapy, diaphragm strength, and cardiovascular and/or respiratory function over time.

Alternatively, sessions can be patient-initiated, giving the patient the autonomy to start a session as needed or when their schedule allows. For instance, a patient may choose to initiate a 20-minute session in the morning before work and a second session in the evening after dinner. This flexibility accommodates individual preferences while maintaining the therapeutic benefits of regular respiratory training.

The system can also combine these approaches, allowing patients to follow pre-programmed schedules while initiating additional sessions as needed. For example, a patient on a pre-programmed regimen of three 15-minute sessions per day might initiate an extra session to address feelings of respiratory discomfort or to practice breathing exercises.

In some embodiments, the implantable pulse generator (IPG) for the phrenic nerve stimulator includes the ability to track and log therapy sessions, providing detailed records of all implemented sessions. This feature enables clinicians to monitor patient progress, evaluate therapy effectiveness, and make informed adjustments to stimulation profiles. Additionally, the IPG can be programmed to implement a series of different therapy sessions, offering a flexible approach to managing respiratory conditioning.

The system can step through a series of therapy sessions in various ways, depending on the programmed criteria. For example, the IPG may progress to the next session in the series upon receiving each successive activation signal from the patient. Alternatively, it could advance through the series after a pre-defined number of iterations for each profile, such as completing X number of activations on stimulation profile A before transitioning to Y activations on stimulation profile B. The progression could also be driven by compliance metrics, where the system evaluates patient adherence to the prescribed therapy and adjusts accordingly. As a further example, the series could be scheduled to progress based on day, time, or other calendar information, ensuring therapy aligns with the patient's daily routine or clinician-defined timeline.

Stimulation profiles can be tailored based on respiratory goals or targets established by a physician or, in some cases, jointly with the patient. For instance, a goal might be based on the maximum achievable tidal volume measured during an office visit. A goal may include, among other things, a minute ventilation goal. Progress toward these goals can be tracked through metrics such as, but not limited to, tidal volume (converted from TTI), blood oxygenation, respiratory rate, waking or arousal rates during therapy for sleep disordered breathing, reported patient comfort, and/or diaphragm contraction strength. The system can display progress via a smartphone user interface, showing milestones or percentage achievement toward the set goals to motivate the patient.

A respiratory conditioning session may begin with, for example, a low stimulation current and a higher stimulation rate stimulation profile. Over the course of the stimulation (e.g., 10-60 minutes), the system incrementally increases the stimulation current to elicit stronger diaphragm contractions while gradually decreasing the stimulation rate. For instance, the session might start at 10 mA with a rate of 20 pulses per minute and progress to 15 mA with a rate of 15 pulses per minute.

To train the patient for slower, deeper breaths, the stimulation profile may increase the length of the rising and falling ramps during a pulse cycle. For example, a typical pulse might have a 1-second rise and fall time. This can be extended to 2 seconds over the course of the session, allowing the patient to achieve more gradual and deeper inhalations and exhalations.

In another example, the system increases the amplitude of the stimulation to elicit more robust diaphragm contractions while decreasing the stimulation rate to emphasize deeper breaths. For instance, the amplitude might increase from 2 mA to 9 mA for typical applications, aligning with common ranges for both cuff and transvenous leads. Although transvenous stimulation could theoretically reach up to 18 mA, doing so may induce Extra Respiratory Sensation (ERS) and is generally avoided. Concurrently, the pulse rate may decrease from 18 to 12 breaths per minute. This combination provides respiratory conditioning benefits over time.

However, some embodiments may provide the delivery of the electrical stimulation to the phrenic nerve at an intensity below an intensity level that activates the diaphragm, such that the electrical stimulation provides a reminder to breath without functionally causing a diaphragm contraction.

In yet another example, a stimulation profile might start with a low energy setting (e.g., 8 mA) and gradually increase the stimulation energy independent of the rate. For instance, the amplitude might increase from 2 mA to 9 mA for typical applications, aligning with common ranges for both cuff and transvenous leads. Concurrently, the pulse rate may decrease from 18 to 12 breaths per minute. This encourages the diaphragm to contract more strongly without altering the rhythm of breathing.

Using TTI (TransThoracic Impedance) as a representative metric for tidal volume, the system could establish a correlation during an office visit. For example, the TTI value might be calibrated to reflect a tidal volume of 500 mL. Training profiles could then aim to increase TTI values over successive sessions, indicating greater tidal volume and improved respiratory function. The system monitors TTI during therapy, adjusts stimulation profiles to optimize tidal volume, and accounts for baseline changes due to factors like fluid retention and/or fluid load.

In various embodiments, baseline parameters may be used for monitoring and adjustment. For example, tidal volume may be used. Tidal volume is the volume of air moved into or out of the lungs during a single breath cycle and is indirectly monitored through TTI signals. During calibration in an office setting, TTI signals are correlated with tidal volume using a conversion equation (e.g., calibration with a sphygmomanometer). TTI values are also corrected for baseline changes caused by fluid retention, ensuring accurate monitoring. For example, if a baseline TTI signal corresponds to 450 mL of tidal volume, the profile may adjust to stimulate stronger diaphragm contractions if TTI values drop below baseline.

As another example, minute ventilation may be used. The system can monitor and adjust minute ventilation (respiratory rate× tidal volume). For example, if minute ventilation falls below a target value during training, the system may increase stimulation intensity or modify rate settings to promote better air movement.

The IPG 12 can be programmed to automatically transition between stimulation profiles based on metrics like tidal volume. For example, if TTI values during a session indicate that the diaphragm has achieved a stronger contraction, the IPG might switch to a profile with slower stimulation rates to emphasize deeper breaths. Alternatively, if TTI values are not improving as expected, the system may increase the amplitude or extend the ramp duration to enhance diaphragm engagement.

Non-sleep time training profiles are distinct from sleep therapy stimulation profiles, which prioritize passive and consistent respiratory support rather than active muscle training. In various embodiments, daytime profiles vary parameters such as the inspiratory-to-expiratory (I:E) ratio, stimulation amplitude, and respiration rate. For example, daytime protocols may begin at intrinsic values, progressively increase amplitude (e.g., mA) while decreasing respiratory rate to promote deeper breathing, and then return to baseline. This variation conditions the diaphragm and improves respiratory strength, and it is not characteristic of the steady, unchanging profiles used during nighttime support. This deliberate separation ensures that each mode of therapy—active daytime training and passive nighttime support—is optimally tailored to its purpose.

The process selects a first stimulation profile (Step 1208) appropriate for the session. The selection of stimulation profiles may vary depending on the patient needs. In some cases, the physician pre-sets the profiles, and the patient uses the controller simply to activate or deactivate the therapy. In other cases, the patient might have the ability to choose from a limited set of profiles, such as one for relaxation and/or vagal tone improvements, another for therapy acclimation improvement, and another for muscle strengthening, etc. based on their immediate needs. Therapy acclimation improvement may be an acclimation to the controlled rate of breathing and/or acclimation to the sensation of neurostimulation, both of which are aspects of sleep disordered breathing therapy. For instance, the user interface may allow the patient to select a “deep breathing training” profile that emphasizes slower stimulation patterns or a “strengthening” profile that provides more intense stimulation. As other examples, the user interface may allow the patient to select a “neurostimulation training” profile that aids in acclimation to the neurostimulation sensations or a “rhythmic training” profile that aids in acclimation to a non-intrinsic breathing rate as caused by neurostimulation. However, these options would be curated by the physician to ensure safety and effectiveness.

The system waits for an activation signal from the patient (Step 1210), ensuring that the patient is ready to proceed. The activation signal may be received via the user interface. When the session begins, the user interface module 163 causes instructions to be provided to the patient, such as breathing guidance, via a mobile device or similar interface (Step 1212). FIG. 5, for example, shown such a breath instruction.

Upon receiving the activation signal, the patient mode controller triggers delivery of electrical stimulation in accordance with 1st conditioning stimulation profile (Step 1214). The stimulation circuitry then generates the electrical pulses in accordance with the selected stimulation profile (Step 1216), triggering diaphragm engagement.

Throughout the session, the patient feedback module 159 collects data on the patient's physiological responses, such as breathing patterns, heart rate, and comfort levels (Step 1218). This feedback is processed by the stimulation profile adjuster 158 (Step 1220) to modify stimulation parameters for future sessions. For example, the system may select a 2_{nd} conditioning stimulation profile 58A(ii) (Step 1221). The adjuster may adjust, among other things, the intensity, frequency, or ramp-up duration to enhance the therapy's effectiveness and improve patient comfort based on this feedback. This feedback may optionally be used to produce an adjusted second conditioning stimulation profile 58B(ii) (Step 1221(b)).

The operational mode controller determines that the patient is in a sleep period (Step 1222). Accordingly, the system enters sleep operational mode (Step 1224), during which the sleep mode controller manages therapy delivery. In this mode, the system accesses a sleep-specific stimulation profile (Step 1226) preconfigured to provide consistent respiratory support.

To enhance safety, the system may include a self-start lock-out feature during the night, preventing the patient from initiating therapy during sleep hours. This ensures that therapy designed for awake conditioning does not interfere with the pre-programmed sleep therapy or disrupt the patient's rest. For instance, the app could display a message during locked-out periods, notifying the patient that therapy can only be initiated during designated awake hours.

The sleep mode controller triggers delivery of electrical stimulation in accordance with 1st sleep stimulation profile (Step 1228). The stimulation circuitry then delivers electrical pulses (Step 1230) in accordance with the 1st sleep stimulation profile, thereby preferably maintaining respiratory stability throughout the sleep period.

Notably, in sleep mode, therapy operates primarily on a preprogrammed schedule, minimizing the need for patient interaction. However, the system remains responsive to the patient's state of arousal. If the patient wakes or exhibits physiological changes indicative of arousal, the system can automatically pause or adjust stimulation as needed to maintain effectiveness without disturbing the patient's sleep.

Feedback collected during both awake and sleep modes informs the stimulation profile adjuster 158 (Step 1220) to ensure ongoing optimization of therapy. For example, if patient feedback during awake sessions reveals discomfort or insufficient diaphragm engagement, the system may adjust the sleep stimulation profile by lowering intensity or modifying timing to ensure more effective sleep therapy. Similarly, feedback indicating successful awake sessions may lead to increased stimulation levels during sleep to further enhance respiratory outcomes.

This system ensures a seamless and adaptive approach to respiratory therapy, leveraging real-time feedback and preprogrammed schedules to deliver safe, effective, and personalized care across both awake and sleep periods.

FIG. 12B shows a detailed swim-lane diagram for a typical flow of a patient control mode. Many of the same steps from FIG. 12A are repeated here, and therefore, are not described again in detail.

During the non-sleep period, the operational mode controller determines that the patient is awake (Step 1202) and transitions the system into patient control mode (Step 1204). In this mode, the patient control mode controller 152 schedules a series of respiratory conditioning sessions (Step 1206) for the awake period. Each scheduled respiration conditioning session is associated with a specific stimulation profile selected from the stimulation profile library.

Although the term “scheduled” is used, it should be understood that the scheduling may be provided based on the medical advice of a medical practitioner. However, it is up to the patient to perform the breathing exercise, and the patient must provide an activation signal before each session. Furthermore, as has been described previously, the patient may make inter-session adjustments to the breathing exercises (e.g., because of pain).

For the first conditioning session, the system selects a first conditioning stimulation profile 58A(i) from the conditioning stimulation profile library 57A (Step 1208(a)). This profile 58A(i) contains predefined parameters such as stimulation intensity, frequency, pulse width, and ramp-up time, tailored to the patient's respiratory needs. The stimulation circuitry then delivers electrical stimulation (Step 1214(a)) in accordance with the selected profile, triggering the desired diaphragm response. This process enables the patient to actively engage in respiratory exercises guided by the system.

Subsequent conditioning sessions during the awake period are similarly managed. For the patient control mode, the system may select a second conditioning stimulation profile 58A(ii) (Step 1208(b)) or additional conditioning stimulation profiles 58A(N) (Step 1208(n)), as appropriate, from the stimulation profile library 57A. Each profile 58A is uniquely configured to incrementally challenge or accommodate the patient's evolving respiratory capabilities or performance. For example, the system may progressively increase stimulation intensity or modify the timing of pulses to optimize therapy outcomes. After selecting each profile 58A, the stimulation circuitry delivers electrical stimulation (Steps 1214(b), 1214(n)) in accordance with the specific parameters of the chosen profile 58A(i), 58A(ii) . . . 58A(N).

The patient control mode allows for flexibility and adaptation based on the patient's feedback and performance. The system can dynamically adjust the sequence and parameters of the stimulation profiles during subsequent sessions to maximize effectiveness and comfort. The ability to schedule multiple conditioning sessions and apply tailored stimulation profiles ensures that the therapy remains personalized and aligned with the patient's progress.

As the non-sleep period concludes, the operational mode controller transitions the system from patient control mode to sleep operational mode (Steps 1222 and 1224). This transition ensures a seamless shift from active respiratory conditioning during awake periods to automated therapy delivery during sleep, as detailed in FIG. 12C.

FIG. 12C shows a detailed swim-lane diagram for a typical flow of a sleep mode of the system. Many of the same steps from FIGS. 12A and 12B are repeated here, and therefore, are not described again in detail.

After entering sleep mode, the sleep mode controller provides one or more stimulation profiles 60A. For example, the IPG 12 may be furnished with a single profile through firmware and/or software and the sleep mode controller may implement the single profile. Alternatively, the IPG 12 may be furnished with a library of stimulation profiles and the sleep mode controller may select from the stimulation profile library 57B. These profiles are specifically configured to maintain stable and effective respiratory support during sleep. For the initial sleep stimulation session, the sleep mode controller selects the first sleep stimulation profile (Step 1226(a)) from the library 57B. This profile includes preconfigured parameters such as intensity, frequency, and pulse width, optimized for sleep therapy. The stimulation circuitry then delivers electrical pulses (Step 1228(a)) in accordance with the selected profile 60A(i), triggering diaphragm activation to support consistent breathing throughout the sleep session.

As the sleep therapy progresses, stimulation may conform to the first stimulation profile or additional stimulation profiles may be utilized, either through reprogramming of the IPG 12 or via a library including multiple profiles 60A(ii) . . . 60(A)(N). The system may access a second sleep stimulation profile or select a second sleep stimulation profile (Step 1226(b)) or subsequent profiles (Step 1226(n)) to adjust the therapy parameters as needed. The selection may be from the stimulation profile library. For each new profile, the stimulation circuitry delivers electrical stimulation (Steps 1228(b), 1228(n)) in accordance with the defined parameters. This sequence allows the system to adapt to the patient's evolving respiratory needs during the sleep period, ensuring both comfort and effectiveness.

The sleep operational mode operates predominantly on a preprogrammed basis, minimizing patient involvement. However, the system remains responsive to changes in the patient's condition, such as state of arousal or physiological feedback. If such changes are detected, the therapy can be adjusted or paused to accommodate the patient's needs.

At the conclusion of the sleep period, the operational mode controller may transition the system back to patient control mode for the non-sleep period (Step 1202). This seamless handoff ensures that the system provides continuous and adaptive therapy across both sleep and awake periods.

This structured approach to sleep therapy leverages tailored stimulation profiles, preprogrammed scheduling, and real-time responsiveness to provide consistent respiratory support during the patient's sleep, improving overall therapy outcomes while maintaining patient comfort.

FIG. 12D shows a detailed swim-lane diagram for a typical flow of a stimulation profile adjusted of the system. Many of the same steps from FIG. 12A-12C are repeated here, and therefore, are not described again in detail.

As described previously, the system includes a stimulation profile adjuster 158 to dynamically modify the stimulation parameters and profiles during patient control mode and sleep mode based on real-time patient feedback and compliance. This process ensures the delivery of tailored respiratory therapy that adapts to the patient's needs and physiological responses.

The process begins with the patient control mode controller 152 selecting the first conditioning stimulation profile 58A(i) from the stimulation profile library 57A (Step 1208). Steps 1212-1216 have already been described, and therefore, are not repeated here.

As the session progresses, the patient feedback module 159 collects data on patient compliance and physiological responses (Step 1218). This feedback is used to evaluate the effectiveness of the stimulation. If the feedback indicates that patient compliance is acceptable and there is no discomfort (step 1218(a)), the system makes no changes to the stimulation profile or parameters (Step 1220(a)).

However, if the feedback indicates patient discomfort or poor compliance—such as overstimulation, insufficient respiratory engagement, or extra respiratory sensation—the stimulation profile adjuster 158 intervenes. Depending on the specific feedback:
1. The stimulation energy for the current session may be adjusted (Step 1220(b)(i)). Thus, if the energy level is too high, the system may reduce intensity to improve comfort. Conversely, if the energy level is too low to elicit the desired diaphragm response, it is increased. For example, energy may be adjusted by one step (e.g., a step in current may be 0.5 mA or 1.0 mA). As an additional example, energy may be adjusted on a percentage basis (e.g., 5%).
2. The selection of subsequent stimulation profiles may be modified (Step 1220(b)(ii)). This adjustment allows future sessions to better match the patient's evolving needs, ensuring incremental progression in respiratory conditioning. For example, the subsequent stimulation profile that is selected may be selected based on a comparative adjustment in energy relative to the most recent stimulation profile. For example, the system may select a stimulation profile that is adjusted by one step (e.g., a step in current may be 0.5 mA or 1.0 mA) or on a percentage basis (e.g., 5%).

Adjustments to stimulation energy are not limited only to peak or average values, but refer generally to the total area under the curve (AUC) of the delivered stimulation waveform. Assuming a constant inspiration-to-expiration ratio, changes to parameters such as current amplitude (which has the largest influence), pulse width, and pulse frequency can be used to modulate the AUC. These adjustments enable precise control over the total energy delivered, thereby tailoring therapy intensity to optimize one or more of patient comfort, diaphragm engagement, upper airway control, patient acclimation, entrainment during sleep disordered breathing therapy, etc.

In cases where further adjustments are necessary, the patient control mode controller 152 selects and applies the second conditioning stimulation profile 58A(ii) (Step 1221) or an adjusted version of the profile 58B(ii) (Step 1221(b)). This new profile may include changes to stimulation energy, timing, or other parameters to address the issues identified in the feedback. The user interface provides revised instructions 59B(ii) instead of 59B(i) (Step 1212). The stimulation circuitry then delivers electrical stimulation in accordance with the updated profile (Steps 1214-1216).

FIG. 12E shows a detailed swim-lane diagram for a typical flow of a stimulation profile adjuster 158 of the system. Many of the same steps from FIG. 12A-12D are repeated here, and therefore, are not described again in detail.

The system extends the functionality of the stimulation profile adjuster 158 to the sleep operational mode. This process integrates data from patient feedback during conditioning sessions and applies adjustments to the sleep stimulation profiles to optimize therapy outcomes (step 1240). A number of different examples of adjustments based on feedback are discussed below (e.g., steps 1240(a)(i), 1240(a)(ii), 1220(b)(ii)).

During sleep mode, the sleep mode controller 44 selects the first sleep stimulation profile 60A(i) from the stimulation profile library (Step 1228(a)). This profile 60A(i) includes preconfigured parameters tailored for the patient's specific respiratory needs during sleep, such as stimulation intensity, frequency, and pulse width. The stimulation circuitry delivers electrical pulses in accordance with the selected profile (Step 1216), ensuring consistent respiratory support throughout the sleep session.

As feedback is collected during sleep therapy, the patient feedback module 159 evaluates the patient's compliance and physiological responses (Step 1218). If the feedback indicates acceptable compliance and no signs of discomfort (step 1218(a)), the system maintains the current stimulation settings without modification (Step 1240(a)(i)). This ensures continuity and stability in therapy delivery when the existing profile meets the patient's needs.

However, if the feedback reveals issues such as patient discomfort overstimulation, or insufficient engagement (step 1218(b)), the stimulation profile adjuster 158 intervenes to modify the therapy parameters (1220(b)(ii)). Depending on the feedback:
1. If adjustments to the current session are required, the system may change from the first sleep stimulation profile 60A(i) to a second sleep stimulation profile 60B(i) during the ongoing sleep session (Step 1228(b)). This allows for immediate corrections to enhance comfort and effectiveness.
2. For long-term improvements, the system modifies subsequent stimulation profiles 60C(i) in the sleep therapy schedule (Step 1240(a)(ii)). For example, the system may adjust parameters such as intensity or timing to better align with the patient's physiological needs based on trends observed during sleep sessions.

In cases where feedback from prior awake-time respiratory conditioning sessions (handled in patient control mode) influences sleep therapy, the system integrates this data to refine sleep stimulation profiles further. For instance, if awake conditioning revealed inadequate diaphragm engagement or overstimulation, the sleep mode stimulation intensity may be reduced, or ramp-up durations extended to ensure smoother transitions into therapy during sleep (Step 1220(b)(ii)). The operation of the adjuster may then return to patient control mode shown in FIG. 12D.

Referring to FIG. 12F, a process of delivering therapy in an AF mode 244 is illustrated. At step 1202, the operational mode controller 156 determines when there is a non-sleep period and transitions the system into patient control mode (Step 1291). Although shown as beginning with 1202 and 1291 to determine a non-sleep period and provide patient controlled conditioning and then proceeding to 1222 and 1296, the IPG 12 may begin with 1222 and 1296, deliver sleep therapy, and then proceed to 1202 at the completion of a sleep therapy to deliver patient controlled conditioning. In some examples, in the AF mode 244, the IPG 12 may deliver only sleep therapy (i.e., bypass entry into the patient control operational mode at step 1291) or may only deliver non-sleep therapy (i.e., bypass entry into the sleep operational mode at step 1296). The steps 1291 and 1296 are substantially the same, respectively, as the steps 1204 and 1224 of the process 1200 with the added feature that in the process 1299, the operational mode controller 21 may determine entry at 1291 and 1296 based at least in part on cardiac metrics (e.g., the cardiac status indicator 63) and/or the therapy mode determined at the step 240 (e.g., the patient therapy mode indicator 62).

In an implementation, respiration signals from the patient may lack indicators of sleep apnea and cardiac signals from the patient may include indicators of atrial fibrillation. These signals may be detected by the IPG 12 and/or a respiration and/or cardiac signal monitor separate from the IPG 12. For instance, a patient may be diagnosed with atrial fibrillation without sleep apnea. Alternatively, the respiration signals may include indicators of sleep apnea and the patient may be diagnosed with atrial fibrillation and sleep apnea. The patient with atrial fibrillation, with or without sleep apnea, may be provided with the IPG 12 for respiratory conditioning. At the stage 1222, the IPG 12 may determine that the patient is in a sleep time period and then, at the stage 1296, the IPG 12 may enter a sleep operational mode and provide phrenic nerve stimulation according to a pre-programmed sleep stimulation profile at the stage 1228. The IPG 12 may access the profile at the stage 1294. The IPG 12 may include a single profile and provide stimulation according to this profile and/or may include a library of profiles and select the profile from the library.

The remainder of the steps in FIG. 12F are substantially the same as described in regard to FIG. 12A, with the exception of the steps 1292, 1293, 1294, and 1295. The steps 1292, 1294, and 1295 are substantially the same, respectively, as the steps 1208, 1226, and 1221 with the added feature that in the process 1299, the operational mode controller 21 may select conditioning profiles at 1292, 1294, and 1295 based at least in part on cardiac metrics (e.g., the cardiac status indicator 63) and/or the therapy mode determined at the step 240 (e.g., the patient therapy mode indicator 62). The step 1293 is substantially the same as the step 1220 with the added feature that in the process 1299, the operational mode controller 21 may adjust a stimulation profile and/or selection of stimulation profiles based at least in part on cardiac metrics (e.g., the cardiac status indicator 63) and/or the therapy mode determined at the step 240 (e.g., the patient therapy mode indicator 62).

Conditioning the diaphragm during the day focuses on improving its strength, endurance, and coordination to enhance respiratory function and support airway stability. This approach leverages muscle memory, enabling the diaphragm to adapt to stimulation patterns that can be reproduced during sleep for continuous therapeutic benefit. As the diaphragm is a sheet muscle that atrophies quickly, respiration conditioning training is essential for improving tonic tone in both the diaphragm and airway muscles, which is especially beneficial for patients with conditions like sleep apnea.

During patient control sessions, the diaphragm may be conditioned to breathe at a specific rate, such as 12 breaths per minute, using initial stimulation parameters such as 2 mA intensity, 20 Hz frequency, and 150 μs pulse width. These parameters are carefully calibrated to engage the diaphragm without causing discomfort. The system monitors the diaphragm's response and adjusts as needed to ensure consistent engagement and effective conditioning.

The resulting conditioned state—a combination of respiration rate and stimulation parameters—is stored for later use. For patients with sleep apnea, this data can be applied to sleep therapy. For instance, if respiration conditioning training achieves a stable respiration rate of 12 breaths per minute with the specified parameters, these settings can be automatically transferred to sleep therapy to replicate the conditioned state during sleep. This integration occurs seamlessly, with the system adapting in real-time to maintain consistency between respiration conditioning and sleep therapy.

After the initial conditioned state is achieved, it is monitored and sustained for 1-2 minutes to reinforce the diaphragm's adaptation. This sustained period ensures the muscle memory is strengthened, allowing the diaphragm to maintain the trained rhythm and engagement over time.

After the sustained period, the stimulus is gradually increased to further enhance patient acclimation, tidal volume, and/or diaphragm strength. Examples of stimulus adjustments include, increasing current amplitude from 2 mA to 3 mA, extending pulse width from 150 μs to 200 μs, adjusting frequency from 20 Hz to 25 Hz, and/or modifying stimulus morphology, such as lengthening the burst duration and smoothing the rising and falling ramps. For instance, ramps might transition from 0.5 seconds to 1 second, creating a more gradual engagement. These changes promote stronger diaphragm contractions, improving both the depth of breathing and respiratory endurance.

Following the stimulus increase, the system reduces the respiration rate to encourage slower, deeper breathing. For example, the rate may decrease from 12 to 11 breaths per minute. To ensure the patient can physiologically accommodate this change, a variable delay is introduced between the stimulus increase and the rate reduction. This delay allows the diaphragm to adjust to the new stimulation parameters before the rate is decreased further.

The new conditioned state—such as 11 breaths per minute with increased stimulation parameters—is then monitored and sustained for another 1-2 minutes. This process ensures the diaphragm adapts fully to the new rhythm and stimulus intensity, reinforcing muscle memory and improving respiratory efficiency.

The cycle of increasing stimulus and reducing respiration rate is repeated to achieve progressively slower, deeper breathing. For example, the system may increase the stimulus again, such as raising the amplitude to 4 mA and lengthening the ramp duration to 2 seconds. The respiration rate may be reduced further, for example, from 11 to 10 breaths per minute. The new conditioned state is monitored and sustained for 1-2 minutes before the next adjustment.

This process continues until the target rate, such as 6-8 breaths per minute, is reached. In some embodiments, ff the patient rolls over, changes posture, and/or abruptly changes posture, the therapy may automatically pause, recalibrate for the new position, and resume the process to maintain therapeutic effectiveness. In various embodiments, the patient could be in an upright position during respiratory conditioning sessions, or, could be in a reclined position.

Throughout the conditioning process, the stimulus morphology evolves to match the patient's progress. Early in therapy, the stimulus may involve short bursts of approximately 2 seconds with rapid rising and falling ramps. As the therapy progresses, the bursts become longer (e.g., 4 seconds), and the ramps lengthen to comprise up to 50% of the burst duration. These changes ensure smoother transitions and natural diaphragm engagement, promoting deeper breathing and enhanced muscle training.

The outcomes of respiration conditioning directly informs sleep therapy. For example, the conditioned state achieved during the day (e.g., 10 breaths per minute at 3 mA, 25 Hz, and 200 μs) can be applied to sleep therapy to replicate the same benefits during sleep. Conversely, feedback from sleep therapy, such as difficulty maintaining tidal volume, can inform adjustments to respiration conditioning training. The system might increase respiration conditioning stimulation intensity or ramp durations to further improve respiratory function and/or patient acclimation to nighttime therapy delivery.

This method of incremental conditioning provides a structured and adaptive approach to diaphragm training, ensuring optimal respiratory function and seamless integration between respiration conditioning and sleep mode therapy. By progressively increasing tidal volume and reducing respiration rate, the system helps patients achieve slower, deeper breathing patterns.

The stimulation circuitry 42 can be coupled to one or more electrodes 34 configured to provide therapy to the patient (e.g., via the leads 14 as described above). For example, the stimulation circuitry 42 can include, or be operably connected to, circuitry components that are configured to generate and provide the therapeutic stimulation. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit and under control of one or more processors (e.g., processor 50) to provide, for example, one or more conditioning or therapeutic pulses.

The data storage 56 can include one or more of non-transitory computer readable media, such as flash memory, solid state memory, magnetic memory, optical memory, cache memory, combinations thereof, and others. The data storage 56 can be configured to store executable instructions and data used for operation of the processor 50. In certain implementations, the data storage can include executable instructions that, when executed, are configured to cause the processor 50 to perform one or more functions.

In some examples, the network interface 52 can facilitate the communication of information between the processor 50 and one or more other devices or entities over a communications network. Such connectivity may enable remote monitoring of the IPG via access to IPG data from a location remotely located from the patient. For example, the network interface 52 can be configured to communicate with a remote computing device such as a remote server or other similar computing device. The network interface 52 can include communications circuitry for transmitting data in accordance with a Bluetooth® wireless standard for exchanging such data over short distances to an intermediary device(s) (e.g., a base station, a “hotspot” device, a smartphone, a tablet, a portable computing device, and/or other devices in proximity of the implantable pulse generator 12). The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain implementations, the user interface 220 can include one or more physical interface devices such as input devices, output devices, and combination input/output devices and a software stack configured to drive operation of the devices. These user interface elements may render visual, audio, and/or tactile content. Thus the user interface 220 may receive input or provide output, thereby enabling a user to interact with the processor 50.

The implantable system 10 can also include at least one battery 54 configured to provide power to one or more components integrated in the implantable system 10. The battery 54 can include a rechargeable multi-cell battery pack. In one example implementation, the battery 54 can include three or more 2200 mAh lithium ion cells that provide electrical power to the other device components within the implantable system 10. For example, the battery 54 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the processor 50.

The implantable system 10 can be coupled to one or more sensors (sensors 18, 19, and/or 22) configured to monitor one or more physiological parameters of the patient. As shown, the sensors may be coupled to the controller via a wired or wireless connection. These sensors may include a variety of external and/or implanted sensors as described herein which may include but are not limited to external and/or implanted flow sensor(s), external and/or implanted acoustic sensor(s), external and/or implanted vibration sensor(s), external and/or implanted electromyography (EMG) sensor(s), a cardiac cycle sensor, an air flow sensor, a pulse oximeter, a chest belt, external and/or implanted heart rate sensor(s), external and/or implanted pressure sensor(s), external and/or implanted actigraphy sensor(s), external peripheral arterial tone sensor(s), external oxygen sensor(s), and/or external and/or implanted transthoracic impedance sensor(s). The flow sensor(s) may be located in or externally near the upper airway of the patient, for example, in a nasal cannula. The acoustic sensor(s) may be microphone(s) located in or externally near the upper airway, on the chest of the patient, or in the thoracic cavity. The vibration sensor(s) may be accelerometer(s) and may be located in or externally near the upper airway. The EMG sensor(s) may be located in the upper airway region (for example, chin or glossal region) to sense stimulation of upper airway muscles, adhered to the neck or chin, and/or implanted on the diaphragm, or adhered to the abdomen of the patient near the diaphragm. The heart rate sensor(s) may be electrocardiogram (ECG) sensors, transthoracic impedance sensors, and/or pulse oximetry sensors. The actigraphy sensor(s) may be accelerometer(s) and/or other motion sensor(s) such as implanted accelerometers or accelerometers adhered to the patient and/or located on a belt or other garment or jewelry as a wearable device. The peripheral arterial tone sensor(s) and pulse oximetry sensor(s) may be external finger-worn devices, such as a clip or glove-tip sensor. The transthoracic impedance sensor(s) may be located on the chest of the patient as electrodes or may be implanted in the thoracic cavity. Sensors referred to as being located externally near the upper airway may be located for example on the head, for example, the chin, neck, or cervical region of the patient's body or may be inserted in the mouth or nose of the patient, for example, with an oral mouthpiece or a nasal cannula.

FIG. 13 illustrates an asynchronous stimulation diagram 1350 and a synchronous stimulation diagram 1360. Each of the diagrams 1350, 1360 depicts actions performed by the system 10 as a function of time, which elapses from left to right in FIG. 13. As shown in FIG. 13, the implantable therapy system 10 may provide the electrical stimulation according to one of various stimulation therapy modes including an asynchronous therapy mode or a synchronous therapy mode. The non-sleep time period mode controller 152 may select the stimulation therapy mode for non-sleep time stimulation and the sleep mode controller 154 may select the stimulation therapy mode for the sleep time stimulation. The system 10 may not implement these modes simultaneously but may operate in one mode at a time with the capability of changing between modes based on programming and/or other settings. Selection of the stimulation therapy mode may occur prior to and/or after the system 10 is implanted in the patient. The system 10 may be configured to change between modes automatically based on programming and/or other settings and/or may change between modes in response to instructions from the external computing device 210 and/or the patient-specific mobile device 215.

Each of the asynchronous sub-mode and the synchronous sub-mode may be used for awake stimulation or for sleeping stimulation. However, while the mode may be the same for an awake period as for a sleep period, the stimulation rate may not be the same.

For example, the pre-determined or pre-programmed asleep asynchronous stimulation rate may be the same or different than the pre-determined or pre-programmed awake asynchronous stimulation rate. Similarly, the rate at which sensed respiration triggers synchronous stimulation in an asleep patient may be rate the same or different than for the awake patient.

In the asynchronous stimulation mode corresponding to the asynchronous stimulation diagram 1350, the system 10 provides the phrenic nerve stimulation at a predetermined and/or pre-programmed stimulation rate based on intrinsic respiration rate sensed in the absence of stimulation and at a time prior to the application of stimulation. The system may then determine the pre-programmed stimulation rate to be equal to the intrinsic rate or 1, 2, 3, 4, or another number of breaths/minute higher or lower than the intrinsic rate. The delivery of stimulation for each breath cycle will then be provided independently (i.e., not triggered by) of respiration sensed in real-time during the provision of therapy.

The time prior to the application of stimulation (1354 or 1358) may be immediately prior, such as within a particular sleeping period or non-sleeping period, or may be a time removed from the particular sleep or non-sleeping period (for example during a surgical recovery period before the patient is released for home use, or at a doctor office visit between uses, etc.). For example, the system 10 may determine a stimulation rate for therapy provided to an awake patient 1352 based on a sensed unstimulated respiration rate (e.g., an awake intrinsic rate) prior to applying therapy while the patient is awake 1354, and may determine a stimulation rate for therapy provided to an asleep patient 1356 based on a sensed unstimulated respiration rate (e.g., an asleep intrinsic rate) prior to applying therapy while the patient is asleep 1358. In an example, the time prior to the application of stimulation (that is, at the times 1354 and 1358) may be subsequent to surgical implantation of the system 10 and during a healing and observation period before the device is activated for at-home use by the patient. For the awake patient, the time prior to the application of stimulation may be a time when the patient is awake but prior to initiation of a respiratory conditioning session. The time prior to the application of stimulation may include a monitoring period (corresponding to times 1354 and 1358) in which the system 10 monitors and detects intrinsic respiration for the purpose of determining an asynchronous stimulation rate. Thus, in the asynchronous sub-mode the implantable treatment system 10 is configured to provide electrical stimulation in response to a preprogrammed or predetermined stimulation rate trigger that excludes respiration sensed concurrently with the applied stimulation therapy. In the synchronous stimulation mode corresponding to the synchronous stimulation diagram 1360, respiration sensed in real-time during stimulation triggers the provision of the phrenic nerve stimulation by the system 10. That is, while a period 1362 prior to applying therapy during which a patient is awake and a period 1364 prior to applying therapy during which a patient is asleep may exist, the patient's intrinsic respiration during these periods 1362, 1364 may not directly control a stimulation rate as there is no stimulation available to trigger during these time periods.

The synchronous stimulation mode provides breathing support and the sensed respiration while stimulation therapy is being provided triggers the stimulation. For example, the system 10 may provide stimulation to an awake patient at a stimulation rate that is based on the patient's sensed respiration while the patient is awake at that time 1366, and may provide stimulation to an asleep patient at a stimulation rate that is based on the patient's sensed respiration while the patient is asleep at that time 1368. In this manner, the stimulation is synchronous with the patient's respiration cycles during stimulation. Thus, the synchronous sub-mode is a respiration triggered stimulation mode.

The synchronous sub-mode may further include providing back-up asynchronous stimulation in response to or based on a lack of a sensed respiration in real-time. For example, the system 10 may determine that there is a lack of a sensed respiration if no respiration signal above a certain threshold is received within a certain threshold period of time (for example, five seconds, 8 second, 10 seconds, 15 seconds, and so forth). Such a back-up asynchronous stimulation may be an automatic feature of the synchronous therapy mode as a patient safety feature so that a patient experiencing a lack of respiration does not remain untreated by the system 10. The system 10 may deliver stimulation in response to detecting the lack of the sensed respiration rather than waiting indefinitely for a sensed respiration trigger. The system 10 may provide the back-up asynchronous stimulation at a pre-determined and/or pre-programmed rate. This rate may be based on previously sensed intrinsic patient respiration or may be based on a population average or other clinically derived effective stimulation rate.

In some examples, a sleeping synchronous sub-mode may include providing back-up asynchronous stimulation but a non-sleeping, or awake, synchronous sub-mode may exclude provision of back-up asynchronous stimulation. In an awake patient, the patient can consciously control and initiate an inspiration in response to and to recover from a period of non-breathing. Further, it may be critical to patient safety not to provide the back-up asynchronous sub-mode in order to prevent a device from initiating an inspiration where an awake patient is apneic for a period of time due to eating, drinking, talking, or other waking activity for which a lack of breath is not harmful and possibly necessary.

FIG. 13 shows asynchronous stimulation provided for both the awake and asleep periods on one timeline and synchronous stimulation provided for both the awake and asleep periods on another timeline, but this representation is for simplicity and brevity only. In various examples, consecutive awake and asleep periods may also use different stimulation therapy modes.

Various embodiments described herein may be implemented at least in part in any conventional computer programming language. For example, some embodiments may be implemented in a procedural programming language (e.g., “C”), or in an object oriented programming language (e.g., “C++”). Other embodiments described herein may be implemented as preprogrammed hardware elements (e.g., application specific integrated circuits, FPGAs, programmable analog circuitry, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments described herein may be implemented as a combination of both software (e.g., a computer program product) and hardware. Still other embodiments described herein are implemented as entirely hardware, or entirely software.

In some implementations, the processor 50 includes one or more processors (or one or more processor cores) that each are configured to perform a series of instructions that result in manipulated data and/or control the operation of the other components of the processor 50. In some implementations, when executing a specific process (e.g., stimulation profile adjustments), the processor 50 can be configured to make specific logic-based determinations based on input data received, and be further configured to provide one or more outputs that can be used to control or otherwise inform subsequent processing to be carried out by the processor 50 and/or other processors or circuitry with which processor 50 is communicatively coupled. Thus, the processor 50 reacts to specific input stimulus in a specific way and generates a corresponding output based on that input stimulus. In some example cases, the processor 50 can proceed through a sequence of logical transitions in which various internal register states and/or other bit cell states internal or external to the processor 50 may be set to logic high or logic low. As referred to herein, the processor 50 can be configured to execute a function where software is stored in a data store coupled to the processor 50, the software being configured to cause the processor 50 to proceed through a sequence of various logic decisions that result in the function being executed.

The various components that are described herein as being executable by the processor 50 can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

As used in this specification and the claims, the singular forms “a,” “an,” and “the” refer to plural referents unless the context clearly dictates otherwise. For example, reference to “the respiration conditioning session” in the singular includes a plurality of respiration conditioning sessions, and reference to “the lead” in the singular includes one or more leads and equivalents known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments described herein.

Although the above discussion discloses various exemplary embodiments, it should be apparent that those skilled in the art can make various modifications that will achieve some of the advantages of the disclosure without departing from the true scope of the disclosure.

1.-88. (canceled)

89. An implantable pulse generator (IPG) for providing respiratory therapy to treat atrial fibrillation for a patient, the IPG configured to couple to at least one implantable stimulation lead configured to deliver electrical stimulation to a phrenic nerve, the IPG comprising:
at least one processor configured to:
determine that the patient is in a sleep time period based at least in part on communication with a clock and a pre-determined sleep time period;
enter a sleep operational mode, the sleep operational mode having one or more sleep treatment sessions, each of the one or more sleep treatment sessions corresponding to a pre-programmed sleep stimulation profile; and
control delivery of the electrical stimulation to the phrenic nerve according to at least one pre-programmed sleep stimulation profile to modulate patient respiration during the sleep operational mode,
wherein the at least one processor is configured to enter the sleep operational mode based on a patient therapy mode indicator comprising an atrial fibrillation therapy indicator.

90. (canceled)

91. The IPG of claim 89, wherein the at least one processor is configured to allow a selection of the patient therapy mode indicator from a group of options comprising a sleep apnea therapy indicator, the atrial fibrillation therapy indicator, and a combination sleep apnea-atrial fibrillation indicator.

92. The IPG of claim 89, wherein the pre-programmed sleep stimulation profile is a first profile targeting atrial fibrillation therapy and comprises different stimulation parameters than a second profile targeting sleep apnea therapy.

93. (canceled)

94. The IPG of claim 92, wherein a stimulation rate of the first profile is less than a stimulation rate of the second profile and wherein a stimulation amplitude of the first profile is greater than a stimulation amplitude of the second profile.

95. (canceled)

96. The IPG of claim 89, wherein the pre-programmed sleep stimulation profile is a first profile targeting atrial fibrillation therapy and comprises equivalent stimulation parameters to a second profile targeting sleep apnea therapy.

97. (canceled)

98. The IPG of claim 89, wherein the at least one pre-programmed sleep stimulation profile specifies a number of discrete stimulation periods provided intermittently for the sleep time period such that the sleep time period includes a plurality of non-stimulation time periods substantially longer than an inhalation-exhalation cycle.

99. The IPG of claim 89, wherein the at least one processor is further configured to:
determine that the patient is in a sleep time period;
access a cardiac status indicator;
bypass the sleep operational mode based on the cardiac status indicator indicating an improvement in AF metrics; and
enter the sleep operational mode based on the cardiac status indicator indicating a deterioration in the AF metrics.

100. (canceled)

101. The IPG of claim 99, comprising at least one cardiac sensor and configured to monitor signals from the at least one cardiac sensor and determine the cardiac status indicator based on the monitored signals, wherein the signals from the at least one cardiac sensor comprise one or more of heart rate signals or electrocardiogram signals.

102.-107. (canceled)

108. The IPG of claim 89, wherein the at least one pre-programmed sleep stimulation profile is configured to cause the at least one processor to deliver the one or more sleep treatment sessions intermittently such that a time between at least two sleep treatment sessions is substantially longer than an inhalation-exhalation cycle for a patient.

109. (canceled)

110. The IPG of claim 89, wherein a duration of at least one sleep treatment session spans a time from a determination that the patient is in the sleep time period to a determination that the patient is aroused based on one or more of a time and a motion detection.

111. The IPG of claim 89, wherein the at least one pre-programmed sleep stimulation profile is configured to provide a stimulation rate of 10-18 bpm and is independent of a patient's intrinsic breathing rate along with a stimulation amplitude of 4-8 mA.

112. The IPG of claim 111, wherein the at least one pre-programmed sleep stimulation profile provides a duty cycle of 5-10% or 5-15%.

113.-130. (canceled)

131. The IPG of claim 89, wherein the IPG is configured to communicatively couple to a patient-specific external computing device via a communications interface and wherein the at least one processor is further configured to receive an activation signal from the patient-specific external computing device responsive to a first user input to the patient-specific external computing device to initiate the electrical stimulation.

132.-133. (canceled)

134. A method of providing respiratory therapy to treat atrial fibrillation for a patient, the method comprising:
determining that the patient is in a sleep time period based at least in part on communication with a clock and a pre-determined sleep time period;
entering a sleep operational mode, the sleep operational mode having one or more sleep treatment sessions, each of the one or more sleep treatment sessions corresponding to a pre-programmed sleep stimulation profile; and
controlling, by an implantable pulse generator (IPG), delivery of electrical stimulation to a phrenic nerve via at least one implantable stimulation lead according to at least one pre-programmed sleep stimulation profile to modulate patient respiration during the sleep operational mode,
wherein the IPG is configured to enter the sleep operational mode based on a patient therapy mode indicator comprising an atrial fibrillation therapy indicator.

135. The method of claim 134, comprising allowing a selection of the patient therapy mode indicator from a group of options comprising a sleep apnea therapy indicator, the atrial fibrillation therapy indicator, and a combination sleep apnea-atrial fibrillation indicator.

136. The method of claim 134, wherein the pre-programmed sleep stimulation profile is a first profile targeting atrial fibrillation therapy and comprises different stimulation parameters than a second profile targeting sleep apnea therapy or stimulation parameters substantially equivalent to the second profile targeting sleep apnea therapy.

137. The method of claim 134, comprising:
determining that the patient is in a sleep time period;
accessing a cardiac status indicator;
bypassing the sleep operational mode based on the cardiac status indicator indicating an improvement in AF metrics; and
entering the sleep operational mode based on the cardiac status indicator indicating a deterioration in the AF metrics.

138. The method of claim 134, wherein the at least one pre-programmed sleep stimulation profile is configured to provide a stimulation rate of 10-18 bpm and is independent of a patient's intrinsic breathing rate along with a stimulation amplitude of 4-8 mA and wherein the at least one pre-programmed sleep stimulation profile provides a duty cycle of 5-10% or 5-15%.

139. The method of claim 134, comprising
monitoring signals from at least one cardiac sensor, and
determining at least one cardiac status indicator based on the monitored signals, wherein the signals from the at least one cardiac sensor comprise one or more of heart rate signals or electrocardiogram signals.

140. The method of claim 134, comprising communicatively coupling, by the IPG, to a patient-specific external computing device via a communications interface, and
receiving an activation signal from the patient-specific external computing device responsive to a first user input to the patient-specific external computing device to initiate the electrical stimulation.
